(19) United States
(12) Patent Application Publication
(10) Pub. No.: US 2007/0275889 A1
(43) Pub. Date: Nov. 29, 2007

(54) Gene Expression Technique

(75) Inventors: Darrell Sleep, Nottingham (GB); Gillian Shuttleworth, Nottingham (GB); Christopher John Arthur Finnis, Nottingham (GB)

Correspondence Address: SYNNESTVEDT & LECHNER, LLP, 1101 MARKET STREET, 26TH FLOOR, PHILADELPHIA, PA, 19107-2950, US

(73) Assignee: Delta Biotechnology Limited, Nottinghom (GB)

(21) Appl. No.: 10/584,424

(22) PCT Filed: Dec. 23, 2004
(86) PCT No.: PCT/GB04/05462

§ 371 Date: Apr. 10, 2007

(30) Foreign Application Priority Data
Dec. 23, 2003 (GB) 0329681.1

(51) Int. Cl. A61K 38/52; C12P 21/04; C12N 1/18

(52) U.S. Cl. 514/12; 435/255.1; 435/320.1; 435/69.9

(57) ABSTRACT

The present invention provides a method for producing heterologous protein comprising: (a) providing a host cell comprising a 2 μm-family plasmid, the plasmid comprising a gene encoding a protein comprising the sequence of a chaperone protein and a gene encoding a heterologous protein; (b) culturing the host cell in a culture medium under conditions that allow the expression of the gene encoding the chaperone protein and the gene encoding a heterologous protein; (c) purifying the thus expressed heterologous protein from the cultured host cell or the culture medium.

### FIELD OF THE INVENTION

The present application relates to gene expression techniques.

### BACKGROUND OF THE INVENTION

The class of proteins known as chaperones have been defined by Hartl (1996, Nature, 381, 571-580) as a protein that binds to and stabilises an otherwise unstable conformer of another protein and, by controlled binding and release, facilitates its correct fate in vivo, be it folding, oligomeric assembly, transport to a particular subcellular compartment, or disposal by degradation.

BiP (also known as GRP78, Ig heavy chain binding protein and Kar2p in yeast) is an abundant ˜70 kDa chaperone of the hsp 70 family, resident in the endoplasmic reticulum (ER), which amongst other functions, serves to assist in transport in the secretory system and fold proteins.

Protein disulphide isomerase (PDI) is a chaperone protein, resident in the ER that is involved in the catalysis of disulphide bond formation during the post-translational processing of proteins.

Studies of the secretion of both native and foreign proteins have shown that transit from the ER to the Golgi is the rate-limiting step. Evidence points to a transient association of the BiP with normal proteins and a more stable interaction with mutant or misfolded forms of a protein. As a result, BiP may play a dual role in solubilising folding precursors and preventing the transport of unfolded and unassembled proteins. Robinson and Wittrup, 1995, Biotechnol. Prog. 11, 171-177, have examined the effect of foreign protein secretion on BiP (Kar2p) and PDI protein levels in Saccharomyces cerevisiae and found that prolonged constitutive expression of foreign secreted proteins reduces soluble BiP and PDI to levels undetectable by Western analysis. The lowering of ER chaperone and foldase levels as a consequence of heterologous protein secretion has important implications for attempts to improve yeast expression/secretion systems.

Expression of chaperones is regulated by a number of mechanisms, including the unfolded protein response (UPR).

Using recombinant techniques, multiple PDI gene copies has been shown to increase PDI protein levels in a host cell (Farquhar et al, 1991, Gene, 108, 81-89).

Co-expression of the gene encoding PDI and a gene encoding a heterologous disulphide-bonded protein was first suggested in WO 93/25676, published on 23 Dec. 1993, as a means of increasing the production of the heterologous protein. WO 93/25676 reports that the recombinant expression of antistasin and tick anticoagulant protein can be increased by co-expression with PDI.

This strategy has been exploited to increase the recombinant expression of other types of protein.

Robinson et al, 1994, Bio/Technology, 12, 381-384 reported that a recombinant additional PDI gene copy in Saccharomyces cerevisiae could be used to increase the recombinant expression of human platelet derived growth factor (PDGF) B homodimer by ten-fold and Schizosacharomyces pombe acid phosphatase by four-fold.

Hayano et al, 1995, FEBS Letters, 377, 505-511 described the co-expression of human lysozyme and PDI in yeast. Increases of around 30-60% in functional lysozyme production and secretion were observed.

Shusta et al, 1998, Nature Biotechnology, 16, 773-777 reported that the recombinant expression of single-chain antibody fragments (scFv) in Saccharomyces cerevisiae could be increased by between 2-8 fold by over-expressing PDI in the host cell.

Bao & Fukuhara, 2001, Gene, 272, 103-110 reported that the expression and secretion of recombinant human serum albumin (rHSA) in the yeast Kluyveromyces lactis could be increased by 15-fold or more by co-expression with an additional recombinant copy of the yeast PDI gene (KlPDI1).

In order to produce co-transformed yeast comprising both a PDI gene and a gene for a heterologous protein, WO 93/25676 taught that the two genes could be chromosomally integrated; one could be chromosomally integrated and one present on a plasmid; each gene could be introduced on a different plasmid; or both genes could be introduced on the same plasmid. WO 93/25676 exemplified expression of antistasin from the plasmid pKH4α2 in yeast strains having a chromosomally integrated additional copy of a PDI gene (Examples 16 and 17); expression of antistasin from the vector K991 with an additional PDI gene copy being present on a multicopy yeast shuttle vector named YEp24 (Botstein et al, 1979, Gene, 8, 17-24) (Example 20); and expression of both the antistasin and the PDI genes from the yeast shuttle vector pC1/1 (Rosenberg et al, 1984, Nature, 312, 77-80) under control of the GAL10 and GAL1 promoters, respectively. Indeed, Robinson and Wittrup, 1995, op. cit., also used the GAL1-GAL10 intergenic region to express erythropoietin and concluded that production yeast strains for the secretion of heterologous proteins should be constructed using tightly repressible, inducible promoters, otherwise the negative effects of sustained secretion (i.e. lowered detectable BiP and PDI) would be dominant after the many generations of cell growth required to fill a large-scale fermenter.

Subsequent work in the field has identified chromosomal integration of transgenes as the key to maximising recombinant protein production.

Robinson et al, 1994, op. cit., obtained the observed increases in expression of PDGF and S. pombe acid phosphatase using an additional chromosomally integrated PDI gene copy. Robinson et al reported that attempts to use the multi-copy 2 μm expression vector to increase PDI protein levels had had a detrimental effect on heterologous protein secretion.

Hayano et al, 1995, op. cit. described the introduction of genes for human lysozyme and PDI into a yeast host each on a separate linearised integration vector, thereby to bring about chromosomal integration.

Shusta et al, 1998, op. cit., reported that in yeast systems, the choice between integration of a transgene into the host chromosome versus the use of episomal expression vectors can greatly affect secretion and, with reference to Parekh & Wittrup, 1997, Biotechnol. Prog., 13, 117-122, that stable integration of the scFv gene into the host chromosome using a δ integration vector was superior to the use of a 2 μm-based expression plasmid. Parekh & Wittrup, op. cit., had previously taught that the expression of bovine pancreatic trypsin inhibitor (BPTI) was increased by an order of magnitude using a δ integration vector rather than a 2 μm-based expression plasmid. The 2 μm-based expression plasmid was said to be counter-productive for the production of heterologous secreted protein.

Bao & Fukuhara, 2001, op. cit., reported that “It was first thought that the KlPDI1 gene might be directly introduced into the multi-copy vector that carried the rHSA expression cassette. However, such constructs were found to severely affect yeast growth and plasmid stability. This confirmed our previous finding that the KlPDI1 gene on a multi-copy vector was detrimental to growth of K. lactis cells (Bao et al, 2000)”. Bao et al, 2000, Yeast, 16, 329-341, as referred to in the above-quoted passage of Bao & Fukuhara, reported that the KlPDI1 gene had been introduced into K. lactis on a multi-copy plasmid, pKan707, and that the presence of the plasmid caused the strain to grow poorly. Bao et al concluded that over-expression of the KlPDI1 gene was toxic to K. lactis cells. In the light of the earlier findings in Bao et al, Bao & Fulcuhara chose to introduce a single duplication of KlPDI1 on the host chromosome.

Against this background, we have surprisingly demonstrated that, contrary to the suggestions in the prior art, when the genes for a chaperone protein and a heterologous protein are co-expressed on a 2 μm-family multi-copy plasmid in yeast, the production of the heterologous protein is substantially increased.

### DESCRIPTION OF THE INVENTION

A first aspect of the present invention provides a method for producing heterologous protein comprising:
(a) providing a host cell comprising a 2 μm-family plasmid, the plasmid comprising a gene encoding a protein comprising the sequence of a chaperone protein and a gene encoding a heterologous protein;
(b) culturing the host cell in a culture medium under conditions that allow the expression of the gene encoding the chaperone protein and the gene encoding a heterologous protein;
(c) purifying the thus expressed heterologous protein from the culture medium; and
(d) optionally, lyophilising the thus purified protein.

In one embodiment, step (c) purifies the thus expressed heterologous protein to a commercially acceptable level of purity or a pharmaceutically acceptable level of purity.

Preferably, the method further comprises the step of formulating the purified heterologous protein with a carrier or diluent, such as a pharmaceutically acceptable carrier or diluent and optionally presenting the thus formulated protein in a unit dosage form.

A second aspect of the present invention provides for the use of a 2 μm-family plasmid as an expression vector to increase the production of a fungal (preferably yeast) or vertebrate heterologous protein by providing a gene encoding the heterologous protein and a gene encoding a protein comprising the sequence of a chaperone protein on the same 2 μm-family plasmid.

A third aspect of the present invention provides a 2 μm-family plasmid comprising a gene encoding a protein comprising the sequence of a chaperone protein and a gene encoding a heterologous protein, wherein if the plasmid is based on the 2 μm plasmid then it is a disintegration vector.

A fourth aspect of the invention provides a host cell comprising a plasmid as defined above.

The present invention relates to recombinantly modified versions of 2 μm-family plasmids.

Certain closely related species of budding yeast have been shown to contain naturally occurring circular double stranded DNA plasmids. These plasmids, collectively termed 2 μm-family plasmids, include pSR1, pSB3 and pSB4 from Zygosaccharomyces rouxii (formerly classified as Zygosaccharomyces bisporus), plasmids pSB1 and pSB2 from Zygosaccharomyces bailii, plasmid pSM1 from Zygosaccharomyces fermentati, plasmid pKD1 from Kluyveromyces drosphilarum, an un-named plasmid from Pichia membranaefaciens (hereinafter “pPM1”) and the 2 μm plasmid and variants (such as Scp1, Scp2 and Scp3) from Saccharomyces cerevisiae (Volkert, et al., 1989, Microbiological Reviews, 53, 299; Murray et al., 1988, J. Mol. Biol. 200, 601; Painting, et al., 1984, J. Applied Bacteriology, 56, 331).

As a family of plasmids these molecules share a series of common features in that they typically possess two inverted repeats on opposite sides of the plasmid, have a similar size around 6-kbp (range 4757 to 6615-bp), three open reading frames, one of which encodes for a site specific recombinase (FLP) and an autonomously replicating sequence (ARS), also known as an origin of replication (ori), located close to the end of one of the inverted repeats. (Futcher, 1988, Yeast, 4, 27; Murray et al., op. cit., and Toh-e et al., 1986, Basic Life Sci. 40, 425). Despite their lack of discernible DNA sequence homology, their shared molecular architecture and the conservation of function of the three open reading frames have demonstrated a common ancestral link between the family members.

Whilst any of the above naturally occurring 2 μm-family plasmids can be used in the present invention, this invention is not limited to the use of naturally occurring 2 μm-family plasmids. For the purposes of this invention, a 2 μm-family plasmid is as described below.

A 2 μm-family plasmid is a circular, double stranded, DNA plasmid. It is typically small, such as between 3,000 to 10,000 bp, preferably between 4,500 to 7000 bp, excluding recombinantly inserted sequences.

A 2 μm-family plasmid typically comprises at least three open reading frames (“ORFs”) that each encodes a protein that functions in the stable maintenance of the 2 μm-family plasmid as a multicopy plasmid. The proteins encoded by the three ORFs can be designated FLP, REP1 and REP2. Where a 2 μm-family plasmid comprises not all three of the ORFs encoding FLP, REP1 and REP2 then ORFs encoding the missing protein(s) should be supplied in trans, either on another plasmid or by chromosomal integration.

A “FLP” protein is a protein capable of catalysing the site-specific recombination between inverted repeat sequences recognised by FLP. The inverted repeat sequences are termed FLP recombination target (FRT) sites and each is typically present as part of a larger inverted repeat (see below). Preferred FLP proteins comprise the sequence of the FLP proteins encoded by one of plasmids pSR1, pSB1, pSB2, pSB3, pSB4, pSM1, pKD1, pPM1 and the 2 μm plasmid, for example as described in Volkert et al, op. cit., Murray et al, op. cit., and Painting et al., op. cit. Variants and fragments of these FLP proteins are also included in the present invention. “Fragments” and “variants” are those which retain the ability of the native protein to catalyse the site-specific recombination between the same FRT sequences. Such variants and fragments will usually have at least 50%, 60%, 70%, 80%, 90%, 95%, 98%, 99%, or more, homology with an FLP protein encoded by one of plasmids pSR1, pSB1, pSB2, pSB3, pSB4, pSM1, pKD1, pPM1 and the 2 μm plasmid. Different FLP proteins can have different FRT sequence specificities. A typical FRT site may comprise a core nucleotide sequence flanked by inverted repeat sequences. In the 2 μm plasmid, the FRT core sequence is 8 nucleotides in length and the flanking inverted repeat sequences are 13 nucleotides in length (Volkert et al, op. cit.). However the FRT site recognised by any given FLP protein may be different to the 2 μm plasmid FRT site.

REP1 and REP2 are proteins involved in the partitioning of plasmid copies during cell division, and may also have a role in the regulation of FLP expression. Considerable sequence divergence has been observed between REP1 proteins from different 2 μm-family plasmids, whereas no sequence alignment is possible between REP2 proteins derived from different 2 μm-family plasmids. Preferred REP1 and REP2 proteins comprise the sequence of the REP1 and REP2 proteins encoded by one of plasmids pSR1, pSB1, pSB2, pSB3, pSB4, pSM1, pKD1, pPM1 and the 2 μm plasmid, for example as described in Volkert et al, op. cit., Murray et al, op. cit., and Painting et al, op. cit. Variants and fragments of these REP1 and REP2 proteins are also included in the present invention. “Fragments” and “variants” of REP1 and REP2 are those which, when encoded by the plasmid in place of the native ORF, do not substantially disrupt the stable multicopy maintenance of the plasmid within a suitable yeast population. Such variants and fragments of REP1 and REP2 will usually have at least 5%, 10%, 20%, 30%, 40%, 50%, 60%, 70%, 80%, 90%, 95%, 98%, 99%, or more, homology with a REP1 and REP2 protein, respectively, as encoded by one of plasmids pSR1, pSB1, pSB2, pSB3, pSB4, pSM1, pKD1, pPM1 and the 2 μm plasmid.

The REP1 and REP2 proteins encoded by the ORFs on the plasmid must be compatible. It is preferred that the REP1 and REP2 proteins have the sequences of REP1 and REP2 proteins encoded by the same naturally occurring 2 μm-family plasmid, such as pSR1, pSB1, pSB2, pSB3, pSB4, pSM1, pKD1, pPM1 and the 2 μm plasmid, or variant or fragments thereof.

A 2 μm-family plasmid typically comprises two inverted repeat sequences. The inverted repeats may be any size, so long as they each contain an FRT site (see above). The inverted repeats are typically highly homologous. They may share greater than 50%, 60%, 70%, 80%, 90%, 95%, 96%, 97%, 98%, 99%, 99.5% or more sequence identity. In a preferred embodiment they are identical. Typically the inverted repeats are each between 200 to 1000 bp in length. Preferred inverted repeat sequences may each have a length of from 200 to 300 bp, 300 to 400 bp, 400 to 500 bp, 500 to 600 bp, 600 to 700 bp, 700 to 800 bp, 800 to 900 bp, or 900 to 1000 bp. Particularly preferred inverted repeats are those of the plasmids pSR1 (959 bp), pSB1 (675 bp), pSB2 (477 bp), pSB3 (391 bp), pSM1 (352 bp), pKD1 (346 bp), the 2 μm plasmid (599 bp), pSB4 or pPM1.

The sequences of the inverted repeats may be varied. However, the sequences of the FRT site in each inverted repeat should be compatible with the specificity of the FLP protein encoded by the plasmid, thereby to enable the encoded FLP protein to act to catalyse the site-specific recombination between the inverted repeat sequences of the plasmid. Recombination between inverted repeat sequences (and thus the ability of the FLP protein to recognise the FRT sites with the plasmid) can be determined by methods known in the art. For example, a plasmid in a yeast cell under conditions that favour FLP expression can be assayed for changes in the restriction profile of the plasmid which would result from a change in the orientation of a region of the plasmid relative to another region of the plasmid. The detection of changes in restriction profile indicate that the FLP protein is able to recognise the FRT sites in the plasmid and therefore that the FRT site in each inverted repeat are compatible with the specificity of the FLP protein encoded by the plasmid.

In a particularly preferred embodiment, the sequences of inverted repeats, including the FRT sites, are derived from the same 2 μm-family plasmid as the ORF encoding the FLP protein, such as pSR1, pSB1, pSB2, pSB3, pSB4, pSM1, pKD1, pPM1 or the 2 μm plasmid.

The inverted repeats are typically positioned with the 2 μm-family plasmid such that the two regions defined between the inverted repeats (e.g. such as defined as UL and US in the 2 μm plasmid) are of approximately similar size, excluding exogenously introduced sequences such as transgenes. For example, one of the two regions may have a length equivalent to at least 40%, 50%, 60%, 70%, 80%, 90%, 95% or more, up to 100%, of the length of the other region.

A 2 μm-family plasmid typically comprises the ORF that encodes FLP and one inverted repeat (arbitrarily termed “IR1” to distinguish it from the other inverted repeat mentioned in the next paragraph) juxtaposed in such a manner that IR1 occurs at the distal end of the FLP ORF, without any intervening coding sequence, for example as seen in the 2 μm plasmid. By “distal end” in this context we mean the end of the FLP ORF opposite to the end from which the promoter initiates its transcription. In a preferred embodiment, the distal end of the FLP ORF overlaps with IR1.

A 2 μm-family plasmid typically comprises the ORF that encodes REP2 and the other inverted repeat (arbitrarily termed “IR2” to distinguish it from IR1 mentioned in the previous paragraph) juxtaposed in such a manner that IR2 occurs at the distal end of the REP2 ORF, without any intervening coding sequence, for example as seen in the 2 μm plasmid. By “distal end” in this context we mean the end of the REP2 ORF opposite to the end from which the promoter initiates its transcription.

In one embodiment, the ORFs encoding REP2 and FLP may be present on the same region of the two regions defined between the inverted repeats of the 2 μm-family plasmid, which region may be the bigger or smaller of the regions (if there is any inequality in size between the two regions).

In one embodiment, the ORFs encoding REP2 and FLP may be transcribed from divergent promoters.

Typically, the regions defined between the inverted repeats (e.g. such as defined as UL and US in the 2 μm plasmid) of a 2 μm-family plasmid may comprise not more than two endogenous genes that encode a protein that functions in the stable maintenance of the 2 μm-family plasmid as a multicopy plasmid. Thus in a preferred embodiment, one region of the plasmid defined between the inverted repeats may comprise not more than the ORFs encoding FLP and REP2; FLP and REP1; or REP1 and REP2, as endogenous coding sequence.

A 2 μm-family plasmid typically comprises an origin of replication (also known as an “autonomously replicating sequence—“ARS”), which is typically bidirectional. Any appropriate ARS sequence can be present. Consensus sequences typical of yeast chromosomal origins of replication may be appropriate (Broach et al, 1982, Cold Spring Harbor Symp. Quant. Biol., 47, 1165-1174; Williamson, Yeast, 1985, 1, 1-14). Preferred ARSs include those isolated from pSR1, pSB1, pSB2, pSB3, pSB4, pSM1, pKD1, pPM1 and the 2 μm plasmid.

Thus, a preferred 2 μm-family plasmid may comprise ORFs encoding FLP, REP1 and REP2, two inverted repeat sequences each inverted repeat comprising an FRT site compatible with the encoded FLP protein, and an ARS sequence. Preferably the FRT sites are derived from the same 2 μm-family plasmid as the sequence of the encoded FLP protein. More preferably the sequences of the encoded REP1 and REP2 proteins are derived from the same 2 μm-family plasmid as each other. Even more preferably, the FRT sites are derived from the same 2 μm-family plasmid as the sequence of the encoded FLP, REP1 and REP2 proteins. Yet more preferably, the sequences of the ORFs encoding FLP, REP1 and REP2, and the sequence of the inverted repeats (including the FRT sites) are derived from the same 2 μm-family plasmid. Furthermore, the ARS site may be derived from the same 2 μm-family plasmid as one or more of the ORFs of FLP, REP1 and REP2, and the sequence of the inverted repeats (including the FRT sites).

The term “derived from” includes sequences having an identical sequence to the sequence from which they are derived. However, variants and fragments thereof, as defined above, are also included. For example, an FLP gene having a sequence derived from the FLP gene of the 2 μm plasmid may have a modified promoter or other regulatory sequence compared to that of the naturally occurring gene.

Additionally or alternatively, an FLP gene having a sequence derived from the FLP gene of the 2 μm plasmid may have a modified nucleotide sequence in the open reading frame which may encode the same protein as the naturally occurring gene, or may encode a modified FLP protein. The same considerations apply to other sequences on a 2 μm-family plasmid having a sequence derived from a particular source.

Optionally, a 2 μm-family plasmid may comprise a region derived from the STB region (also known as REP3) of the 2 μm plasmid, as defined in Volkert et al, op. cit. The STB region in a 2 μm-family plasmid of the invention may comprise two or more tandem repeat sequences, such as three, four, five or more. Alternatively, no tandem repeat sequences may be present. The tandem repeats may be any size, such as 10, 20, 30, 40, 50, 60 70, 80, 90, 100 bp or more in length. The tandem repeats in the STB region of the 2 μm plasmid are 62 bp in length. It is not essential for the sequences of the tandem repeats to be identical. Slight sequence variation can be tolerated. It may be preferable to select an STB region from the same plasmid as either or both of the REP1 and REP2 ORFs. The STB region is thought to be a cis-acting element and preferably is not transcribed.

Optionally, a 2 μm-family plasmid may comprise an additional ORF that encodes a protein that functions in the stable maintenance of the 2 μm-family plasmid as a multicopy plasmid. The additional protein can be designated RAF or D. ORFs encoding the RAF or D gene can be seen on, for example, the 2 μm plasmid and pSM1. Thus a RAF or D ORF can comprise a sequence suitable to encode the protein product of the RAF or D gene ORFs encoded by the 2 μm plasmid or pSM1, or variants and fragments thereof. Thus variants and fragments of the protein products of the RAF or D genes of the 2 μm plasmid or pSM1 are also included in the present invention. “Fragments” and “variants” of the protein products of the RAF or D genes of the 2 μm plasmid or pSM1 are those which, when encoded by the 2 μm plasmid or pSM1 in place of the native ORF, do not disrupt the stable multicopy maintenance of the plasmid within a suitable yeast population. Such variants and fragments will usually have at least 5%, 10%, 20%, 30%, 40%, 50%, 60%, 70%, 80%, 90%, 95%, 98%, 99%, or more, homology with the protein product of the RAF or D gene ORFs encoded by the 2 μm plasmid or pSM1.

A naturally occurring 2 μm-family plasmid may be preferred. A naturally occurring 2 μm-family plasmid is any plasmid having the features defined above, which plasmid is found to naturally exist in yeast, i.e. has not been recombinantly modified to include heterologous sequence. Preferably the naturally occurring 2 μm-family plasmid is selected from pSR1 (Accession No. X02398), pSB3 (Accession No. X02608) or pSB4 as obtained from Zygosaccharomyces rouxii, pSB1 or pSB2 (Accession No. NC_{—}002055 or M18274) both as obtained from Zygosaccharomyces bailli, pSM1 (Accession No. NC_{—}002054) as obtained from Zygosaccharomyces fermentati, pKD1 (Accession No. X03961) as obtained from Kluyveromyces drosophilarum, pPM1 from Pichia membranaefaciens or, most preferably, the 2 μm plasmid (Accession No. NC_{—}001398 or J01347) as obtained from Saccharomyces cerevisiae. Accession numbers in this paragraph refer to NCBI deposits.

The 2 μm plasmid (FIG. 1) is a 6,318-bp double-stranded DNA plasmid, endogenous in most Saccharomyces cerevisiae strains at 60-100 copies per haploid genome. The 2 μm plasmid comprises a small unique (US) region and a large unique (UL) region, separated by two 599-bp inverted repeat sequences. Site-specific recombination of the inverted repeat sequences results in inter-conversion between the A-form and B-form of the plasmid in vivo (Volkert & Broach, 1986, Cell, 46, 541). The two forms of 2 μm differ only in the relative orientation of their unique regions.

While DNA sequencing of a cloned 2 μm plasmid (also known as Scp1) from Saccharomyces cerevisiae gave a size of 6,318-bp (Hartley and Donelson, 1980, Nature, 286, 860), other slightly smaller variants of 21 in, Scp2 and Scp3, are known to exist as a result of small deletions of 125-bp and 220-bp, respectively, in a region known as STB (Cameron et al., 1977, Nucl. Acids Res., 4, 1429: Kikuchi, 1983, Cell, 35, 487 and Livingston & Hahne, 1979, Proc. Natl. Acad. Sci. USA, 76, 3727). In one study about 80% of natural Saccharomyces strains from around the world contained DNA homologous to 2 μm (by Southern blot analysis) (Hollenberg, 1982, Current Topics in Microbiology and Immunobiology, 96, 119). Furthermore, variation (genetic polymorphism) occurs within the natural population of 2 μm plasmids found in S. cerevisiae and S. carlsbergensis, with the NCBI sequence (accession number NC_{—}001398) being one example.

The 2 μm plasmid has a nuclear localisation and displays a high level of mitotic stability (Mead et al, 1986, Molecular & General Genetics, 205, 417). The inherent stability of the 2 μm plasmid results from a plasmid-encoded copy number amplification and partitioning mechanism, which can be compromised during the development of chimeric vectors (Futcher & Cox, 1984, J. Bacteriol., 157, 283; Bachmair & Ruis, 1984, Monatshefte für Chemie, 115, 1229). A yeast strain, which contains a 2 μm plasmid is known as [cir⁺], while a yeast strain which does not contain a 2 μm plasmid is known as [cir⁰].

The US-region of the 2 μm plasmid contains the REP2 and FLP genes, and the UL-region contains the REP1 and D (also known as RAF) genes, the STB-locus and the origin of replication (Broach & Hicks, 1980, Cell, 21, 501; Sutton & Broach, 1985, Mol. Cell. Biol., 5, 2770). The Flp recombinase binds to FRT-sites (Flp Recognition Target) within the inverted repeats to mediate site-specific recombination, which is essential for natural plasmid amplification and control of plasmid copy number in vivo (Senecoff et al, 1985, Proc. Natl. Acad. Sci. U.S., 82, 7270; Jayaram, 1985, Proc. Natl. Acad. Sci. U.S.A., 82, 5875). The copy number of 2 μm-family plasmids can be significantly affected by changes in Flp recombinase activity (Sleep et al, 2001, Yeast, 18, 403; Rose & Broach, 1990, Methods Enzymol., 185, 234). The Rep1 and Rep2 proteins mediate plasmid segregation, although their mode of action is unclear (Sengupta et al, 2001, J. Bacteriol., 183, 2306). They also repress transcription of the FLP gene (Reynolds et al, 1987, Mol. Cell. Biol., 7, 3566).

The FLP and REP2 genes of the 2 μm plasmid are transcribed from divergent promoters, with apparently no intervening sequence defined between them. The FLP and REP2 transcripts both terminate at the same sequence motifs within the inverted repeat sequences, at 24-bp and 178-bp respectively after their translation termination codons (Sutton & Broach, 1985, Mol. Cell. Biol., 5, 2770).

In the case of FLP, the C-terminal coding sequence also lies within the inverted repeat sequence. Furthermore, the two inverted repeat sequences are highly conserved over 599-bp, a feature considered advantageous to efficient plasmid replication and amplification in vivo, although only the FRT-sites (less than 65-bp) are essential for site-specific recombination in vitro (Senecoff et al, 1985, Proc. Natl. Acad. Sci. U.S.A., 82, 7270; Jayaram, 1985, Proc. Natl. Acad. Sci. U.S.A., 82, 5875; Meyer-Leon et al, 1984, Cold Spring Harbor Symposia On Quantitative Biology, 49, 797). The key catalytic residues of Flp are arginine-308 and tyrosine-343 (which is essential) with strand-cutting facilitated by histidine-309 and histidine 345 (Prasad et al, 1987, Proc. Natl. Acad. Sci. U.S.A., 84, 2189; Chen et al, 1992, Cell, 69, 647; Grainge et al, 2001, J. Mol. Biol., 314, 717).

Two functional domains are described in Rep2. Residues 15-58 form a Rep1-binding domain, and residues 59-296 contain a self-association and STB-binding region (Sengupta et al, 2001, J. Bacteriol., 183, 2306).

Chimeric or large deletion mutant derivatives of 2 μm which lack many of the essential functional regions of the 2 μm plasmid but retain the functional cis element ARS and STB, cannot effectively partition between mother and daughter cells at cell division. Such plasmids can do so if these functions are supplied in trans, by for instance the provision of a functional 2 μm plasmid within the host, such as a [cir⁺] host.

Genes of interest have previously been inserted into the UL-region of the 2 μm plasmid. For example, see plasmid pSAC3U1 in EP 0 286 424 and the plasmid shown in FIG. 2, which includes a β-lactamase gene (for ampicillin resistance), a LEU2 selectable marker and an oligonucleotide linker, the latter two of which are inserted into a unique SnaBI-site within the UL-region of the 2 μm-like disintegration vector, pSAC3 (see EP 0 286 424). The E. coli DNA between the XbaI-sites that contains the ampicillin resistance gene is lost from the plasmid shown in FIG. 2 after transformation into yeast. This is described in Chinery & Hinchliffe, 1989, Curr. Genet., 16, 21 and EP 0 286 424, where these types of vectors are designated “disintegration vectors”. Further polynucleotide insertions can be made in a NotI-site within a linker (Sleep et al, 1991, Biotechnology (NY), 9, 183).

Alternative insertion sites in 2 μm plasmid are known in the art, including those described in Rose & Broach (1990, Methods Enzymol., 185, 234-279), such as plasmids pCV19, pCV20, CVₙₑₒ, which utilise an insertion at EcoRI in FLP, plasmids pCV21, pGT41 and pYE which utilise EcoRI in D as the insertion site, plasmid pHKB52 which utilises PstI in D as the insertion site, plasmid pJDB248 which utilises an insertion at PstI in D and EcoRI in D, plasmid pJDB219 in which PstI in D and EcoRI in FLP are used as insertion sites, plasmid G18, plasmid pAB18 which utilises an insertion at ClaI in FLP, plasmids pGT39 and pA3, plasmids pYT11, pYT14 and pYT11-LEU which use PstI in D as the insertion site, and plasmid PTY39 which uses EcoRI in FLP as the insertion site.

Other 2 μm plasmids include pSAC3, pSAC3U1, pSAC3U2, pSAC300, pSAC310, pSAC3C1, pSAC3PL1, pSAC3SL4, and pSAC3SC1 are described in EP 0 286 424 and Chinery & Hinchliffe (1989, Curr. Genet., 16, 21-25) which also described PstI, EagI or SnaBI as appropriate 2 μm insertion sites. Further 2 μm plasmids include pAYE255, pAYE316, pAYE443, pAYE522 (Kerry-Williams et al, 1998, Yeast, 14, 161-169), pDB2244 (WO 00/44772), and pAYE329 (Sleep et al, 2001, Yeast, 18, 403-421).

In one preferred embodiment, one or more genes are inserted into a 2 μm-family plasmid within an untranscribed region around the ARS sequence. For example, in the 2 μm plasmid obtained from S. cerevisiae, the untranscribed region around the ARS sequence extends from end of the D gene to the beginning of ARS sequence. Insertion into SnaBI (near the origin of replication sequence ARS) is described in Chinery & Hinchliffe, 1989, Curr. Genet., 16, 21-25. The skilled person will appreciate that gene insertions can also be made in the untranscribed region at neighbouring positions to the SnaBI site described in Chinery & Hinchliffe.

In another preferred embodiment, REP2 and FLP genes in a 2 μm-family plasmid each have an inverted repeat adjacent to them, and one or more genes are inserted into a 2 μm-family plasmid within the region between the first base after the last functional codon of either the REP2 gene or the FLP gene and the last base before the FRT site in the inverted repeat adjacent to said gene. The last functional codon of either a REP2 gene or a FLP gene is the codon in the open reading frame of the gene that is furthest downstream from the promoter of the gene whose replacement by a stop codon will lead to an unacceptable loss of multicopy stability of the plasmid, as defined herein. Thus, disruption of the REP2 or FLP genes at any point downstream of the last functional codon in either gene, by insertion of a polynucleotide sequence insertion, deletion or substitution will not lead to an unacceptable loss of multicopy stability of the plasmid.

For example, the REP2 gene of the 2 μm plasmid can be disrupted after codon 59 and that the FLP gene of the 2 μm plasmid can be disrupted after codon 344, each without a loss of multicopy stability of the plasmid. The last functional codon in equivalent genes in other 2 μm-family plasmids can be determined routinely by making mutants of the plasmids in either the FLP or REP2 genes and following the tests set out herein to determine whether the plasmid retains multicopy stability.

One can determined whether a plasmid retains multicopy stability using test such as defined in Chinery & Hinchliffe (1989, Curr. Genet., 16, 21-25). For yeast that do not grow in the non-selective media (YPD, also designated YEPD) defined in Chinery & Hinchliffe (1989, Curr. Genet., 16, 21-25) other appropriate non-selective media might be used. Plasmid stability may be defined as the percentage cells remaining prototrophic for the selectable marker after a defined number of generations. The number of generations will preferably be sufficient to show a difference between a control plasmid, such as pSAC35 or pSAC310, or to shown comparable stability to such a control plasmid. The number of generations may be 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more. Higher numbers are preferred. The acceptable plasmid stability might be 1%, 2%, 3%, 4%, 5%, 10%, 15%, 20%, 25%, 30%, 40%, 50%, 60%, 70%, 75%, 80%, 85%, 90%, 95%, 96%, 97%, 98%, 99%, 99.9% or substantially 100%. Higher percentages are preferred. The skilled person will appreciate that, even though a plasmid may have a stability less than 100% when grown on non-selective media, that plasmid can still be of use when cultured in selective media. For example plasmid pDB2711 as described in the examples is only 10% stable when the stability is determined accordingly to test of Example 1, but provides a 15-fold increase in recombinant transferrin productivity in shake flask culture under selective growth conditions.

Thus one or more gene insertions may occur between the first base after the last functional codon of the REP2 gene and the last base before the FRT site in an inverted repeat adjacent to said gene, more preferably between the first base of the inverted repeat and the last base before the FRT site, even more preferably at a position after the translation termination codon of the REP2 gene and before the last base before the FRT site.

Additionally or alternatively one or more gene insertions may occur between the first base after the last functional codon of the FLP gene and the last base before the FRT site in an inverted repeat adjacent to said gene, preferably between the first base of the inverted repeat and the last base before the FRT site, more preferably between the first base after the end of the FLP coding sequence and the last base before the FRT site, such as at the first base after the end of the FLP coding sequence.

In one preferred embodiment, where the 2 μm-family plasmid is based on the 2 μm plasmid of S. cerevisiae, it is a disintegration vector as known in the art (for example, see EP 286 424, the contents of which are incorporated herein by reference). A disintegration vector may be a 2 μm plasmid vector comprising a DNA sequence which is intended to be lost by recombination, three 2 μm FRT sites, of which one pair of sites is in direct orientation and the other two pairs are in indirect orientation, and a DNA sequence of interest (such as an E. coli origin of replication and bacterial selectable marker), the said sequence to be lost being located between the said sites which are in direct orientation.

Thus, the sequence to be lost may comprise a selectable marker DNA sequence.

A preferred disintegration vector comprises a complete 2 μm plasmid additionally carrying (i) a bacterial plasmid DNA sequence necessary for propagation of the vector in a bacterial host; (ii) an extra 2 μm FRT site; and a selectable marker DNA sequence for yeast transformation; the said bacterial plasmid DNA sequence being present and the extra FRT site being created at a restriction site, such as XbaI, in one of the two inverted repeat sequences of the 2 μm plasmid, the said extra FRT site being in direct orientation in relation to the endogenous FRT site of the said one repeat sequence, and the bacterial plasmid DNA sequence being sandwiched between the extra FRT site and the endogenous FRT site of the said one repeat sequence. In a preferred disintegration vector, all bacterial plasmid DNA sequences are sandwiched as said. A particularly preferred 2 μm plasmid vector has substantially the configuration of pSAC3 as shown in EP 286 424.

The term “disintegration vector” as used herein also includes plasmids as defined in U.S. Pat. No. 6,451,559, the contents of which are incorporated herein by reference. Thus a disintegration vector may be a 2 μm vector that, other than DNA sequence encoding non-yeast polypeptides, contains no bacterial (particularly E. coli) origin of replication, or more preferably no bacterial (particularly E. coli) sequence and preferably all DNA in said vector, other than DNA sequence encoding non-yeast polypeptides, is yeast-derived DNA.

The term “chaperone” as used herein refers to a protein that binds to and stabilises an otherwise unstable conformer of another protein, and by controlled binding and release, facilitates its correct fate in vivo, be it folding, oligomeric assembly, transport to a particular subcellular compartment, or disposal by degradation. Accordingly a chaperone is also a protein that is involved in protein folding, or which has chaperone activity or is involved in the unfolded protein response. Chaperone proteins of this type are known in the art, for example in the Stanford Genome Database (SGD), http:://db.yeastgenome.org. Preferred chaperones are eukaryotic chaperones, especially preferred chaperones are yeast chaperones, including AHA1, CCT2, CCT3, CCT4, CCT5, CCT6, CCT7, CCT8, CNS1, CPR3, CPR6, ERO1, EUG1, FMO1, HCH1, HSP10, HSP12, HSP104, HSP26, HSP30, HSP42, HSP60, HSP78, HSP82, JEM1, MDJ1, MDJ2, MPD1, MPD2, PDI1, PFD1, ABC1, APJ1, ATP11, ATP12, BTT1, CDC37, CPR7, HSC82, KAR2, LHS1, MGE1, MRS11, NOB1, ECM10, SSA1, SSA2, SSA3, SSA4, SSC1, SSE2, SIL1, SLS1, ORM1, ORM2, PER1, PTC2, PSE1, UBI4 and HAC1 or a truncated intronless HAC1 (Valkonen et al. 2003, Applied Environ. Micro., 69, 2065)

A chaperone useful in the practice of the present invention may be:
a heat shock protein, such as a protein that is a member of the hsp70 family of proteins (including Kar2p, SSA and SSB proteins, for example proteins encoded by SSA1, SSA2, SSA3, SSA4, SSB1 and SSB2), a protein that is a member of the HSP90-family, or a protein that is a member of the HSP40-family or proteins involved in their modulation (e.g. Sil1p), including DNA-J and DNA-J-like proteins (e.g. Jem1p, Mdj2p);
a protein that is a member of the karyopherin/importin family of proteins, such as the alpha or beta families of karyopherin/importin proteins, for example the karyopherin beta protein PSE1;
a protein that is a member of the ORMDL family described by Hjelmqvist et al, 2002, Genome Biology, 3(6), research0027.1-0027.16, such as Orm2p.
a protein that is naturally located in the endoplasmic reticulum or elsewhere in the secretory pathway, such as the golgi. For example, a protein that naturally acts in the lumen of the endoplasmic reticulum (ER), particularly in secretory cells. such as PDI
a protein that is transmembrane protein anchored in the ER, such as a member of the ORMDL family described by Hjelmqvist et al, 2002, supra, (for example, Orm2p);
a protein that acts in the cytosol, such as the hsp70 proteins, including SSA and SSB proteins, for example protein production SSA1, SSA2, SSA3, SSA4, SSB1 and SSB2;
a protein that acts in the nucleus, the nuclear envelope and/or the cytoplasm, such as Pse1p;
a protein that is essential to the viability of the cell, such as PDI or an essential karyopherin protein, such as Pse1p;
a protein that is involved in sulphydryl oxidation or disulphide bond formation, breakage or isomerization, or a protein that catalyses thiol:disulphide interchange reactions in proteins, particularly during the biosynthesis of secretory and cell surface proteins, such as protein disulphide isomerases (e.g. Pdi1p, Mpd1p), homologues (e.g. Eug1p) and/or related proteins (e.g. Mpd2p, Fmo1p, Ero1p);
a protein that is involved in protein synthesis, assembly or folding, such as PDI and Ssa1p;
a protein that binds preferentially or exclusively to unfolded, rather than mature protein, such as the hsp70 proteins, including SSA and SSB proteins, for example proteins encoded by SSA1, SSA2, SSA3, SSA4, SSB1 and SSB2;
a protein that prevents aggregation of precursor proteins in the cytosol, such as the hsp70 proteins, including SSA and SSB proteins, for example proteins encoded by SSA1, SSA2, SSA3, SSA4, SSB1 and SSB2;
a protein that binds to and stabilises damaged proteins, for example Ssa1p;
a protein that is involved in the unfolded protein response or provides for increased resistance to agents (such as tunicamycin and dithiothreitol) that induce the unfolded protein response, such as a member of the ORMDL family described by Hjelmqvist et al, 2002, supra (for example, Orm2p) or proteins involved in the response to stress (e.g. Ubi4p);
a protein that is a co-chaperone and/or a protein indirectly involved in protein folding and/or the unfolded protein response (e.g. hsp104p, Mdj1p);
a protein that is involved in the nucleocytoplasmic transport of macromolecules, such as Pse1p;
a protein that mediates the transport of macromolecules across the nuclear membrane by recognising nuclear location sequences and nuclear export sequences and interacting with the nuclear pore complex, such as PSE1;
a protein that is able to reactivate ribonuclease activity against RNA of scrambled ribonuclease as described in as described in EP 0 746 611 and Hillson et al, 1984, Methods Enzymol., 107, 281-292, such as PDI;
a protein that has an acidic pI (for example, 4.0-4.5), such as PDI;
a protein that is a member of the Hsp70 family, and preferably possesses an N-terminal ATP-binding domain and a C-terminal peptide-binding domain, such as Ssa1p.
a protein that is a peptidyl-prolyl cis-trans isomerases (e.g. Cpr3p, Cpr6p);
a protein that is a homologue of known chaperones (e.g. Hsp10p);
a protein that is a mitochondrial chaperone (e.g Cpr3p);
a protein that is a cytoplasmic or nuclear chaperone (e.g Cns1p);
a protein that is a membrane-bound chaperone (e.g. Orm2p, Fmo1p);
a protein that has chaperone activator activity or chaperone regulatory activity (e.g. Aha1p, Hac1p, Hch1p);
a protein that transiently binds to polypeptides in their immature form to cause proper folding transportation and/or secretion, including proteins required for efficient translocation into the endoplasmic reticulum (e.g. Lhs1p) or their site of action within the cell (e.g. Pse1p);
a protein that is a involved in protein complex assembly and/or ribosome assembly (e.g. Atp11p, PseIp, Nob1p);
a protein of the chaperonin T-complex (e.g. Cct2p); or
a protein of the prefoldin complex (e.g. Pfd1p).

A preferred chaperone is protein disulphide isomerase (PDI) or a fragment or variant thereof having an equivalent ability to catalyse the formation of disulphide bonds within the lumen of the endoplasmic reticulum (ER). By “PDI” we include any protein having the ability to reactivate the ribonuclease activity against RNA of scrambled ribonuclease as described in EP 0 746 611 and Hillson et al, 1984, Methods Enzymol., 107, 281-292.

PDI is an enzyme which typically catalyzes thiol:disulphide interchange reactions, and is a major resident protein component of the ER lumen in secretory cells. A body of evidence suggests that it plays a role in secretory protein biosynthesis (Freedman, 1984, Trends Biochem. Sci., 9, 438-41) and this is supported by direct cross-linking studies in situ (Roth and Pierce, 1987, Biochemistry, 26, 4179-82). The finding that microsomal membranes deficient in PDI show a specific defect in cotranslational protein disulphide (Bulleid and Freedman, 1988, Nature, 335, 649-51) implies that the enzyme functions as a catalyst of native disulphide bond formation during the biosynthesis of secretory and cell surface proteins. This role is consistent with what is known of the enzyme's catalytic properties in vitro; it catalyzes thiol:disulphide interchange reactions leading to net protein disulphide formation, breakage or isomerization, and can typically catalyze protein folding and the formation of native disulphide bonds in a wide variety of reduced, unfolded protein substrates (Freedman et al., 1989, Biochem. Soc. Symp., 55, 167-192). PDI also functions as a chaperone since mutant PDI lacking isomerase activity accelerates protein folding (Hayano et al, 1995, FEBS Letters, 377, 505-511). Recently, sulphydryl oxidation, not disulphide isomerisation was reported to be the principal function of Protein Disulphide Isomerase in S. cerevisiae (Solovyov et al., 2004, J. Biol. Chem., 279 (33) 34095-34100). The DNA and amino acid sequence of the enzyme is known for several species (Scherens et al, 1991, Yeast, 7, 185-193; Farquhar et al, 1991, Gene, 108, 81-89; EP074661; EP0293793; EP0509841) and there is increasing information on the mechanism of action of the enzyme purified to homogeneity from mammalian liver (Creighton et al, 1980, J. Mol. Biol., 142, 43-62; Freedman et al, 1988, Biochem. Soc. Trans., 16, 96-9; Gilbert, 1989, Biochemistry, 28, 7298-7305; Lundstrom and Holmgren, 1990, J. Biol. Chem., 265, 9114-9120; Hawkins and Freedman, 1990, Biochem. J, 275, 335-339). Of the many protein factors currently implicated as mediators of protein folding, assembly and translocation in the cell (Rothman, 1989, Cell, 59, 591-601), PDI has a well-defined catalytic activity.

The deletion or inactivation of the endogenous PDT gene in a host results in the production of an inviable host. In other words, the endogenous PDI gene is an “essential” gene.

PDT is readily isolated from mammalian tissues and the homogeneous enzyme is a homodimer (2×57 kD) with characteristically acidic pI (4.0-4.5) (Hillson et al, 1984, op. cit.). The enzyme has also been purified from wheat and from the alga Chlamydomonas reinhardii (Kaska et al, 1990, Biochem. J, 268, 63-68), rat (Edman et al, 1985, Nature, 317, 267-270), bovine (Yamauchi et al, 1987, Biochem. Biophys. Res. Comm., 146, 1485-1492), human (Pihlajaniemi et al, 1987, EMBO J., 6, 643-9), yeast (Scherens et al, supra; Farquhar et al, op. cit.) and chick (Parkkonen et al, 1988, Biochem. J, 256, 1005-1011). The proteins from these vertebrate species show a high degree of sequence conservation throughout and all show several overall features first noted in the rat PDI sequence (Edman et al., 1985, op. cit.).

Preferred PDI sequences include those from humans and those from yeast species, such as S. cerevisiae.

A yeast protein disulphide isomerase precursor, PDI1, can be found as Genbank accession no. CAA42373 or BAA00723. It has the following sequence of 522 amino acids:

| | |
|---|---|
| 1 | mkfsagavls wsslllassv faqqeavape dsavvklatd sfneyiqshd lvlaeffapw |
| 61 | cghckninape yvkaaetlve knitlaqidc tenqdlcmeh nipgfpslki fknsdvnnsi |
| 121 | dyegprtaea ivqfmikqsq pavavvadlp aylanetfvt pvivqsgkid adfnatfysm |
| 181 | ankhfndydf vsaenadddf kislyipsam depvvyngkk adiadadvfe kwlqvealpy |
| 241 | fgeidgsvfa qyvesgipig ylfyndeeel eeykplftel akknrglmnf vsidarkfgr |
| 301 | hagnlnmkeq fplfaihdmt edlkyglpql seeafdelsd kivieskale slvkdflkgd |
| 361 | aspivksqei fenqdssvfq lvgknhdeiv ndpkkdvlvi yyapwcghck riaptyqela |
| 421 | dtyanatsdv liakldhten dvrgvviegy ptiviypggk ksesvvyqgs rsldsltdfi |
| 481 | kenghfdvdg kalyeeaqek aaeeadadae ladeedaihd el |

An alternative yeast protein disulphide isomerase sequence can be found as Genbank accession no. CAA38402. It has the following sequence of 530 amino acids

| | |
|---|---|
| 1 | mkfsagavls wsslllassv faqqeavape dsavvklatd sfneyiqshd lvlaeffapw |
| 61 | cghckninape yvkaaetlve knitlaqidc tenqdlcmeh nipgfpslki fknrdvnnsi |
| 121 | dyegprtaea ivqfmikqsq pavavvadlp aylanetfvt pvivqsgkid adfnatfysm |
| 181 | ankhfndydf vsaenadddf klsiylpsam depvvyngkk adiadadvfe kwlqvealpy |
| 241 | fgeidgsvfa qyvesgipig ylfyndeeel eeykplftel akknrglmnf vsidarkfgr |
| 301 | hagnlnmkeq fplfaihdmt edlkyglpql seeafdelsd kivieskale slvkdflkgd |
| 361 | aspivksqei fenqdssvfq lvgknhdeiv ndpkkdvlvl yyapwcghck riaptygela |
| 421 | dtyanatsdv liakldhten dvrgvviegy ptivlypggk ksesvvyqgs rsldslfdfi |
| 481 | kenghfdvdg kalyeeaqek aaeeaeadae aeadadaela deedaihdel |

The following alignment of these sequences (the sequence of Genbank accession no. CAA42373 or BAA00723 first, the sequence of Genbank accession no. CAA38402 second) shows that the differences between these two sequences are a single amino acid difference at position 114 (highlighted in bold) and that the sequence defined by Genbank accession no. CAA38402 contains the additional amino acids EADAEAEA at positions 506-513.

| | |
|---|---|
| 1 | mkfsagavls wsslllassv faqqeavape dsavvklatd sfneyiqshd lvlaeffapw |
| 1 | mkfsagavls wsslllassv faqqeavape dsavvklatd sfneyiqshd lvlaeffapw |
| 61 | cghcknmape yvkaaetlve knitlaqidc tenqdlcmeh nipgfpslki fknsdvnnsi |
| 61 | cghcknmape yvkaaetlve knitlaqidc tenqdlcmeh nipgfpslki fknrdvnnsi |
| 121 | dyegprtaea ivqfmikqsq pavavvadlp aylanetfvt pvivqsgkid adfnatfysm |
| 181 | dyegprtaea ivqfmikqsq pavavvadlp aylanetfvt pvivqsgkid adfnatfysm |
| 181 | ankhfndydf vsaenadddf kisiylpsam depvvyngkk adiadadvfe kwlqvealpy |
| 181 | ankhfndydf vsaenadddf kisiylpsam depvvyngkk adiadadvfe kwlqvealpy |
| 241 | fgeidgsvfa qyvesglplg ylfyndeeel eeykplftel akknrglmnf vsidarkfgr |
| 241 | fgeidgsvfa qyvesglplg ylfyndeeel eeykplftel akknrglmnf vsidarkfgr |
| 301 | hagnlnmkeq fplfaihdmt edlkyglpql seeafdelsd kivleskaie slvkdflkgd |
| 301 | hagnlnmkeq fplfaihdmt edlkyglpql seeafdelsd kivleskaie slvkdflkgd |
| 361 | aspivksqei fenqdssvfq lvgknhdeiv ndpkkdvlvl yyapwcghck rlaptyqela |
| 361 | aspivksqei fenqdssvfq lvgknhdeiv ndpkkdvlvl yyapwcghck rlaptyqela |
| 421 | dtyanatsdv liakldhten dvrgvviegy ptivlypggk ksesvvyqgs rsldslfdfi |
| 421 | dtyanatsdv liakldhten dvrgvviegy ptivlypggk ksesvvyqgs rsldslfdfi |
| 481 | kenghfdvdg kalyeeaqek aaeea***** ***dadaela deedaihdel |
| 481 | kenghfdvdg kalyeeaqek aaeeaeadae aeadadaela deedaihdel |

Variants and fragments of the above PDI sequences, and variants of other naturally occurring PDI sequences are also included in the present invention. A “variant”, in the context of PDI, refers to a protein wherein at one or more positions there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” typically has at least 25%, at least 50%, at least 60% or at least 70%, preferably at least 80%, more preferably at least 90%, even more preferably at least 95%, yet more preferably at least 99%, most preferably at least 99.5% sequence identity to the polypeptide from which it is derived.

The percent sequence identity between two polypeptides may be determined using suitable computer programs, as discussed below. Such variants may be natural or made using the methods of protein engineering and site-directed mutagenesis as are well known in the art.

A “fragment”, in the context of PDI, refers to a protein wherein at one or more positions there have been deletions. Thus the fragment may comprise at most 5, 10, 20, 30, 40 or 50%, typically up to 60%, more typically up to 70%, preferably up to 80%, more preferably up to 90%, even more preferably up to 95%, yet more preferably up to 99% of the complete sequence of the full mature PDI protein. Particularly preferred fragments of PDI protein comprise one or more whole domains of the desired protein.

A fragment or variant of PDI may be a protein that, when expressed recombinantly in a host cell, can complement the deletion of the endogenously encoded PDI gene in the host cell, such as S. cerevisiae, and may, for example, be a naturally occurring homolog of PDI, such as a homolog encoded by another organism, such as another yeast or other fungi, or another eukaryote such as a human or other vertebrate, or animal or by a plant.

Another preferred chaperone is SSA1 or a fragment or variant thereof having an equivalent chaperone-like activity. SSA1, also known as YG100, is located on chromosome I of the S. cerevisiae genome and is 1.93-kbp in size.

One published protein sequence of SSA1 is as follows:

MSKAVGIDLGTTYSCVAHFANDRVDIIANDQGNRTTPSFVAFTDTERLIG
DAAKNQAAMNPSNTVFDAKRLIGRNFNDPEVQADMKHFPFKLIDVDGKPQ
IQVEFKGETKNFYPEQISSMVLGKMKETAESYLGAKVNDAVVTVPAYFND
SQRQATKDAGTIAGLNVLRIINEPTAAAIAYGLDKKGKEEHVLIFDLGGG
TFDVSLLFIEDGIFEVKATAGDTHLGGEDFDNRLVNHFIQEFKRKNKKDL
STNQRALRRLRTACERAKRTLSSSAQTSVEIDSLFEGIDFYTSITRARFE
ELCADLFRSTLDPVEKVLRDAKLDKSQVDEIVLVGGSTRIPKVQKLVTDY
FNGKEPNRSINPDEAVAYGAAVQAAILTGDESSKTQDLLLLDVAPLSLGI
ETAGGVMTKLIPRNSTISTKKFEIFSTYADNQPGVLIQVFEGERAKTKDN
NLLGKFELSGIPPAPRGVPQIEVTFDVDSNGILNVSAVEKGTGKSNKITI
TNDKGRLSKEDIEKMVAEAEKFKEEDEKESQRIASKNQLESIAYSLKNTI
SEAGDKLEQADKDTVTKKAEETISWLDSNTTASKEEFDDKLKELQDIANP
IMSKLYQAGGAPGGAAGGAPGGFPGGAPPAPEAGPTVEEVD

A published coding sequence for SSA1 is as follows, although it will be appreciated that the sequence can be modified by degenerate substitutions to obtain alternative nucleotide sequences which encode an identical protein product:

ATGTCAAAAGCTGTCGGTATTGATTTAGGTACAACATACTCGTGTGTTGC
TCACTTTGCTAATGATCGTGTGGACATTATTGCCAACGATCAAGGTAACA
GAACCACTCCATCTTTTGTCGCTTTCACTGACACTGAAAGATTGATTGGT
GATGCTGCTAAGAATCAAGCTGCTATGAATCCTTCGAATACCGTTTTCGA
CGCTAAGCGTTTGATCGGTAGAAACTTCAACGACCCAGAAGTGCAGGCTG
ACATGAAGCACTTCCCATTCAAGTTGATCGATGTTGACGGTAAGCCTCAA
ATTCAAGTTGAATTTAAGGGTGAAACCAAGAACTTTACCCCAGAACAAAT
CTCCTCCATGGTCTTGGGTAAGATGAAGGAAACTGCCGAATCTTACTTGG
GAGCCAAGGTCAATGACGCTGTCGTCACTGTCCCAGCTTACTTCAACGAT
TCTCAAAGACAAGCTACCAAGGATGCTGGTACCATTGCTGGTTTGAATGT
CTTGCGTATTATTAACGAACCTACCGCCGCTGCCATTGCTTACGGTTTGG
ACAAGAAGGGTAAGGAAGAACACGTCTTGATTTTCGACTTGGGTGGTGGT
ACTTTCGATGTCTCTTTGTTGTTCATTGAAGACGGTATCTTTGAAGTTAA
GGCCACCGCTGGTGACACCCATTTGGGTGGTGAAGATTTTGACAACAGAT
TGGTCAACCACTTCATCCAAGAATTCAAGAGAAAGAACAAGAAGGACTTG
TCTACCAACCAAAGAGCTTTGAGAAGATTAAGAACCGCTTGTGAAAGAGC
CAAGAGAACTTTGTCTTCCTCCGCTCAAACTTCCGTTGAAATTGACTCTT
TGTTCGAAGGTATCGATTTCTACACTTCCATCACCAGAGCCAGATTCGAA
GAATTGTGTGCTGACTTGTTCAGATCTACTTTGGACCCAGTTGAAAGGTC
TTGAGAGATGCTAAATTGGACAAATCTCAAGTCGATGAAATTGTCTTGGT
CGGTGGTTCTACCAGAATTCCAAAGGTCCAAAAATTGGTCACTGACTACT
TCAACGGTAAGGAACCAAACAGATCTATCAACCCAGATGAAGCTGTTGCT
TACGGTGCTGCTGTTCAAGCTGCTATTTTGACTGGTGACGAATCTTCCAA
GACTCAAGATCTATTGTTGTTGGATGTCGCTCCATTATCCTTGGGTATTG
AAACTGCTGGTGGTGTCATGACCAAGTTGATTCCAAGAAACTCTACCATT
TCAACAAAGAAGTTCGAGATCTTTTCCACTTATGCTGATAACCAACCAGG
TGTCTTGATTCAAGTCTTTGAGGTGAAAGAGCCAAGACTAAGGACAAACA
ACTTGTTGGGTAAGTTCGAATTGAGTGGTATTCCACCAGCTCCAAGAGGT
GTCCCACAAATTGAAGTCACTTTCGATGTCGACTCTAACGGTATTTTGAT
GTTTCCGCCGTCGAAAAGGGTACTGGTAAGTCTAACAAGATCACTATTAC
CAACGACAAGGGTAGATTGTCCAAGGAAGATATCGAAGATGGTTGCTGAA
GCCGAAAAATTCAAGGAAGAAGATGAAAAGGAATCTCAAAGAATTGCTTC
CAAGAACCAATTGGAATCCATTGCTTACTCTTTGAAGAACACCATTTCTG
AAGCTGGTGACAAATTGGAACAAGCTGACAAGGACACCGTCACCAAGAAG
GCTGAAGAGACTATTTCTTGGTTAGACAGCAACACCACTGCCAGCAAGGA
AGATTCGATGACAGTTGAAGGAGTTGCAAGACATTGCCAACCCAATCATG
TCTAAGTTGTACCAAGCTGGTGGTGCTCCAGGTGGCGCTGCAGGTGGTGC
TCCAGGCGGTTTCCCAGGTGGTGCTCCTCCAGCTCCAGAGGCTGAAGGTC
CAACCGTTGAAGAAGTTGATTAA

The protein Ssa1p belongs to the Hsp70 family of proteins and is resident in the cytosol. Hsp70s possess the ability to perform a number of chaperone activities; aiding protein synthesis, assembly and folding; mediating translocation of polypeptides to various intracellular locations, and resolution of protein aggregates (Becker & Craig, 1994, Eur. J. Biochem. 219, 11-23). Hsp70 genes are highly conserved, possessing an N-terminal ATP-binding domain and a C-terminal peptide-binding domain. Hsp70 proteins interact with the peptide backbone of, mainly unfolded, proteins. The binding and release of peptides by hsp70 proteins is an ATP-dependent process and accompanied by a conformational change in the hsp70 (Becker & Craig, 1994, supra).

Cytosolic hsp70 proteins are particularly involved in the synthesis, folding and secretion of proteins (Becker & Craig, 1994, supra). In S. cerevisiae cytosolic hsp70 proteins have been divided into two groups; SSA (SSA 1-4) and SSB (SSB 1 and 2) proteins, which are functionally distinct from each other. The SSA family is essential in that at least one protein from the group must be active to maintain cell viability (Becker & Craig, 1994, supra). Cytosolic hsp70 proteins bind preferentially to unfolded and not mature proteins. This suggests that they prevent the aggregation of precursor proteins, by maintaining them in an unfolded state prior to being assembled into multimolecular complexes in the cytosol and/or facilitating their translocation to various organelles (Becker & Craig, 1994, supra). SSA proteins are particularly involved in posttranslational biogenesis and maintenance of precursors for translocation into the endoplasmic reticulum and mitochondria (Kim et al., 1998, Proc. Natl. Acad. Sci. USA. 95, 12860-12865; Ngosuwan et al., 2003, J. Biol. Chem. 278 (9), 7034-7042). Ssa1p has been shown to bind damaged proteins, stabilising them in a partially unfolded form and allowing refolding or degradation to occur (Becker & Craig, 1994, supra; Glover & Lindquist, 1998, Cell. 94, 73-82).

Demolder et al, 1994, J. Biotechnol., 32, 179-189 reported that over-expression of SSA1 in yeast provided for increases in the expression of a recombinant chromosomally integrated gene encoding human interferon-β. There is no suggestion that increases in heterologous gene expression could be achieved if SSA1 and human interferon-β were to be encoded by recombinant genes on the same plasmid. In fact, in light of more recent developments in the field of over-expression of chaperones in yeast (e.g. Robinson et al, 1994, op. cit.; Hayano et al, 1995, op. cit., Shusta et al, 1998, op. cit, Parekh & Wittrup, 1997, op. cit.; Bao & Fukuhara, 2001, op. cit.; and Bao et al, 2000, op. cit) the skilled person would have been disinclined to express SSA1 from a 2 μm-family plasmid at all, much less to express both SSA1 and a heterologous protein from a 2 μm-family plasmid in order to increase the expression levels of a heterologous protein.

Variants and fragments of SSA1 are also included in the present invention. A “variant”, in the context of SSA1, refers to a protein having the sequence of native SSA1 other than at one or more positions where there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” of SSA1 typically has at least 25%, at least 50%, at least 60% or at least 70%, preferably at least 80%, more preferably at least 90%, even more preferably at least 95%, yet more preferably at least 99%, most preferably at least 99.5% sequence identity to the sequence of native SSA1.

The percent sequence identity between two polypeptides may be determined using suitable computer programs, as discussed below. Such variants may be natural or made using the methods of protein engineering and site-directed mutagenesis as are well known in the art.

A “fragment”, in the context of SSA1, refers to a protein having the sequence of native SSA1 other than for at one or more positions where there have been deletions. Thus the fragment may comprise at most 5, 10, 20, 30, 40 or 50%, typically up to 60%, more typically up to 70%, preferably up to 80%, more preferably up to 90%, even more preferably up to 95%, yet more preferably up to 99% of the complete sequence of the full mature SSA1 protein. Particularly preferred fragments of SSA1 protein comprise one or more whole domains of the desired protein.

A fragment or variant of SSA1 may be a protein that, when expressed recombinantly in a host cell, such as S. cerevisiae, can complement the deletion of the endogenously encoded SSA1 gene (or homolog thereof) in the host cell and may, for example, be a naturally occurring homolog of SSA1, such as a homolog encoded by another organism, such as another yeast or other fingi, or another eukaryote such as a human or other vertebrate, or animal or by a plant.

Another preferred chaperone is PSE1 or a fragment or variant thereof having equivalent chaperone-like activity.

PSE1, also known as KAP121, is an essential gene, located on chromosome XIII.

A published protein sequence for the protein pse1p is as follows:

MSALPEEVNRTLLQIVQAFASPDNQIRSVAEKALSEEWITENNIEYLLTF
LAEQAAFSQDTTVAALSAVLFRKLALKAPPSSKLMIMSKNITHIRKEVLA
QIRSSLLKGFLSERADSIRHKLSDAIAECVQDDLPAWPELLQALIESLKS
GNPNFRESSFRILTTVPYLITAVDINSILPIFQSGFTDASDNVKIAAVTA
FVGYFKQLPKSEWSKLGILLPSLLNSLPRFLDDGKDDALASVFESLIELV
ELAPKLFKDMFDQIIQFTDMVIKNKDLEPPARTTALELLTVFSENAPQMC
KSNQNYGQTLVMVTLIMMTEVSIDDDDAAEWIESDDTDDEEEVTYDHARQ
ALDRVALKLGGEYLAAPLFQYLQQMITSTEWRERFAAMMALSSAAEGCAD
VLIGEIPKILDMVIPLINDPHPRVQYGCCNVLGQISTDFSPFIQRTAHDR
ILPALISKLTSECTSRVQTHAAAALVNFSEFASKDILEPYLDSLLTNLLV
LLQSNKLYVQEQALTTIAFIAEAAKNKFIKYYDTLMPLLLNVLKVNNKDN
SVLKGKCMECATLIGFAVGKEKFHEHSQELISILVALQNSDIDEDDALRS
YLEQSWSRICRILGDDFVPLLPIVIPPLLITAKATQDVGLIEEEEAANFQ
QYPDWDVVQVQGKHIAIHTSVLDDKVSAMELLQSYATLLRGQFAVYVKEV
MEEIALPSLDFYLHDGVRAAGATLIPILLSCLLAATGTQNEELVLLWHKA
SSKLIGGLMSEPMPEITQVYHNSLVNGIKVMGDNCLSEDQLAAFTKGVSA
NLTDTYERMQDRHGDGDEYNENIDEEEDFTDEDLLDEINKSIAAVLKTTN
GHYLKNLENIWPMINTFLLDNEPILVIFALVVIGDLIQYGGEQTASMKNA
FIPKVTECLISPDARIRQAASYIIGVCAQYAPSTYADVCIPTLDTLVQIV
DFPGSKLEENRSSTENASAAIAKILYAYNSNIPNVDTYTANWFKTLPTIT
DKEAASFNYQFLSQLIENNSPIVCAQSNISAVVDSVIQALNERSLTEREG
QTVISSVKKLLGFLPSSDAMAIFNRYPADIMEKVHKWFA*

A published nucleotide coding sequence of PSE1 is as follows, although it will be appreciated that the sequence can be modified by degenerate substitutions to obtain alternative nucleotide sequences which encode an identical protein product:

ATGTCTGCTTTACCGGAAGAAGTTAATAGAACATTACTTCAGATTGTCCA
GGCGTTTGCTTCCCCTGACAATCAATACGTTCTGTAGCTGAGAAGGCTCT
TAGTGAAGAATGGATTACCGAAAACAATATTGAGTATCTTTTAACTTTTT
TGGCTGAACAAGCCGCTTTCTCCCAAGATACAACAGTTGCAGCATTATCT
GCTGTTCTGTTTAGAAAATTAGCATTAAAAGCTCCCCCTTCTTCGAAGCT
TATGATTATGTCCATATCACACATATTAGGAAAGAAGTTCTTGCACAAAT
TCGTTCTTCATTGTTAAAAGGGTTTTTGTCGGAAAGAGCTGATTCAATTA
GGCACAAACTATCTGATGCTATTGCTGAGTGTGTTCAAGACGACTTACCA
GCATGGCCAGAATTACTACAAGCTTTAATAGAGTCTTTAAAAAGCGGTAA
CCCAAATTTTAGAGAATCCAGTTTTAGAATTTTGACGACTGTACCTTATT
TAATTACCGCTGTTGACATCAACAGTATCTTACCAATTTTTCAATCAGGC
TTTACTGATGCAAGTGATAATGTCAAAATTGCTGCAGTTACGGCTTTCGT
GGGTTATTTTAAGCAACTACCAAAATCTGAGTGGTCCAAGTTAGGTATTT
TATTACCAAGTCTTTTGAATAGTTTACCAAGATTTTTAGATGATGGTAAG
GACGATGCCCTTGCATCAGTTTTTGAATCGTTAATTGAGTTGGTGGAATT
GGCACCAAAACTATTCAAGGATATGTTTGACCAAATAATACAATTCACTG
ATATGGTTATAAAAAATAAGGATTTAGAACCTCCAGCAAGAACCACAGCA
CTCGAACTGCTAACCGTTTTCAGCGAGAACGCTCCCCAAATGTGTAAATC
GAACCAGAATTACGGGCAAACTTTAGTGATGGTTACTTTAATCATGATGA
CGGAGGTATCCATAGATGATGATGATGCAGCAGAATGGATAGAATCTGAC
GATACCGATGATGAAGAGGAAGTTACATATGACCACGCTCGTCAAGCTCT
TGATCGTGTTGCTTTAAAGCTGGGTGGTGAATATTTGGCTGCACCATTGT
TCCAATATTTACAGCATGATCACATCAACCGAATGGAGAGAAAGATTCGC
GGCCATGATGGCACTTTCCTCTGCAGCTGAGGGTTGTGCTGATGTTCTGA
TCGGCGAGATCCCAAAAATCCTGGATATGGTAATTCCCCTCATCAACGAT
CCTCATCCAAGAGTACAGTATGGATGTTGTAATGTTTTGGGTCAAATATC
TACTGATTTTTCACCATTCATTCAAAGAACTGCACACGATAGAATTTTGC
ACACACGATAGAATTTTGCCGGCTTTAATATCTAAACTAACGTCAGAATG
CACCTCAAGAGTTCAAACGCACGCCGCAGCGGCTCTGGTTAACTTTTCTG
AATTCGCTTCGAAGGATATTCTTGAGCCTTACTTGGATAGTCTATTGACA
AATTTATTAGTTTTATTACAAGCAACAAACTTTACGTACAGGAACAGGCC
CTAACAACCATTGCATTTATTGCTGAAGCTGCAAAGAATAAATTTATCAA
GTATTACGATACTCTAATGCCATTATTATTAAATGTTTTGAAGGTTAACA
ATAAAGATAATAGTGTTTGAAAGGTAAATGTATGGAATGTGCAACTCTGA
TTGGTTTTGCCGTTGGTAAGGAAAAATTTCATGAGCACTCTCAAGAGCTG
ATTTCTATATTGGTCGCTTTACAAAACTCAGATATCGATGAAGATGATGC
GCTCAGATCATACTTAGAACAAAGTTGGAGCAGGATTTGCCGAATTCTGG
GTGATGATTTTGTTCCGTTGTTACCGATTGTTATACCACCCCTGCTAATT
ACTGCCAAAGCAACGCAAGACGTCGGTTTAATTGAAGAAGAAGAAGCAGC
AAATTTCCAACAATATCCAGATTGGGATGTTGTTCAAGTTCAGGGAAACA
CATTGCTATTCACACATCCGTCCTTGACGATAAAGTATCAGCAATGGAGC
TATTACAAAGCTATGCGACACTTTTAAGAGGCCAATTTGCTGTATATGTT
AAAGAAGTAATGGAAGAAATAGCTCTACCATCGCTTGACTTTTACCTACA
TGACGGTGTTCGTGCTGCAGGAGCAACTTTAATTCCTATTCTATTATCTT
GTTTACTTGCAGCCACCGGTACTCAAAACGAGGAATTGGTATTGTTGTGG
CATAAAGCTTCGTCTAAACTAATCGGAGGCTTAATGTCAGAACCAATGCC
AGAAATCACGCAAGTTTATCACAACTCGTTAGTGAATGGTATTAAAGTCA
TGGGTGACAATTGCTTAAGCGAAGACCAATTAGCGGCATTTACTAAGGGT
GTCTCCGCCAACTTAACTGACACTTACGAGGATGCAGGATCGCCATGGTG
ATGGTGATGAATATAATGAAAATATTGATGAAGAGGAAGACTTTACTGAC
GAAGATCTTCTCGATGAAATCAACAAGTCTATCGCGGCCGTTTTGAAAAC
CACAAATGGTCATTATCTAAAGAATTTGGAGAATATATGGCCTATGATAA
ACACATTCCTTTTAGATAATGAACCAATTTTAGTCATTTTTGCATTAGTA
GTGATTGGTGACTTGATTCAATATGGTGGCGAACAAACTGCTAGCATGAA
GAACGCATTTATTCCAAAGGTTACCGAGTGCTTGATTTCTCCTGACGCTC
GTATTCGCCAAGCTGCTTCTTATATAATCGGTGTTTGTGCCCAATACGCT
CCATCTACATATGCTGACGTTTGCATACCGACTTTAGATACACTTGTTCA
GATTGTCGATTTTCCAGGCTCCAAACTGGAAGAAAATCGTTCTTCAACAG
AGAATGCCAGTGCAGCCATCGCCAAAATTCTTTATGCATACAATTCCAAC
ATTCCTAACGTAGACACGTACACGGCTAATTGGTTCAAAACGTTACCAAC
AATAACTGACAAGAAGCTGCCTCATTCAACTATCAATTTTTGAGTCAATT
GATTGAAAATAATTCGCCAATTGTGTGTGCTCAATCTAATATCTCCGCTG
TAGTTGATTCAGTCATACAAGCCTTGAATGAGAGAAGTTTGACCGAAAGG
GAAGGCCAAACGGTGATAAGTTCAGTTAAAAAGTTGTTGGGATTTTTGCC
TTCTAGTGATGCTATGGCAATTTTCAATAGATATCCAGCTGATATTATGG
AGAAAGTACATAAATGGTTTGCATAA

The PSE1 gene is 3.25-kbp in size. Pse1p is involved in the nucleocytoplasmic transport of macromolecules (Seedorf & Silver, 1997, Proc. Natl. Acad. Sci. USA. 94, 8590-8595). This process occurs via the nuclear pore complex (NPC) embedded in the nuclear envelope and made up of nucleoporins (Ryan & Wente, 2000, Curr. Opin. Cell Biol. 12, 361-371). Proteins possess specific sequences that contain the information required for nuclear import, nuclear localisation sequence (NLS) and export, nuclear export sequence (NES) (Pemberton et al., 1998, Curr. Opin. Cell Biol. 10, 392-399). Pse1p is a karyopherin/importin, a group of proteins, which have been divided up into α and β families. Karyopherins are soluble transport factors that mediate the transport of macromolecules across the nuclear membrane by recognising NLS and NES, and interact with and the NPC (Seedorf & Silver, 1997, supra; Pemberton et al., 1998, supra; Ryan & Wente, 2000, supra). Translocation through the nuclear pore is driven by GTP hydrolysis, catalysed by the small GTP-binding protein, Ran (Seedorf & Silver, 1997, supra). Pse1p has been identified as a karyopherin β. 14 karyopherin β proteins have been identified in S. cerevisiae, of which only 4 are essential. This is perhaps because multiple karyopherins may mediate the transport of a single macromolecule (Isoyama et al., 2001, J. Biol. Chem. 276 (24), 21863-21869). Pse1p is localised to the nucleus, at the nuclear envelope, and to a certain extent to the cytoplasm. This suggests the protein moves in and out of the nucleus as part of its transport function (Seedorf & Silver, 1997, supra). Pse1p is involved in the nuclear import of transcription factors (Isoyama et al., 2001, supra; Ueta et al., 2003, J. Biol. Chem. 278 (50), 50120-50127), histones (Mosammaparast et al., 2002, J. Biol. Chem. 277 (1), 862-868), and ribosomal proteins prior to their assembly into ribosomes (Pemberton et al., 1998, supra). It also mediates the export of mRNA from the nucleus. Karyopherins recognise and bind distinct NES found on RNA-binding proteins, which coat the RNA before it is exported from the nucleus (Seedorf & Silver, 1997, Pemberton et al., 1998, supra).

As nucleocytoplasmic transport of macromolecules is essential for proper progression through the cell cycle, nuclear transport factors, such as pse1p are novel candidate targets for growth control (Seedorf & Silver, 1997, supra).

Overexpression of Pse1p (protein secretion enhancer) in S. cerevisiae has also been shown to increase endogenous protein secretion levels of a repertoire of biologically active proteins (Chow et al., 1992; J. Cell Sci. 101 (3), 709-719). There is no suggestion that increases in heterologous gene expression could be achieved if PSE1 and a heterologous protein were both to be encoded by recombinant genes on the same plasmid. In fact, in light of more recent developments in the over-expression of chaperones in yeast (e.g. Robinson et al, 1994, op. cit.; Hayano et al, 1995, op. cit., Shusta et al, 1998, op. cit; Pareld & Wittrup, 1997, op. cit.; Bao & Fukuhara, 2001, op. cit.; and Bao et al, 2000, op. cit) the skilled person would not have attempted to over-express PSE1 from a 2 μm-family plasmid at all, much less to express both PSE1 and a heterologous protein from a 2 μm-family plasmid in order to increase the expression levels of a heterologous protein.

Variants and fragments of PSE1 are also included in the present invention. A “variant”, in the context of PSE1, refers to a protein having the sequence of native PSE1 other than for at one or more positions where there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” of PSE1 typically has at least 25%, at least 50%, at least 60% or at least 70%, preferably at least 80%, more preferably at least 90%, even more preferably at least 95%, yet more preferably at least 99%, most preferably at least 99.5% sequence identity to the sequence of native PSE1.

The percent sequence identity between two polypeptides may be determined using suitable computer programs, as discussed below. Such variants may be natural or made using the methods of protein engineering and site-directed mutagenesis as are well known in the art.

A “fragment”, in the context of PSE1, refers to a protein having the sequence of native PSE1 other than for at one or more positions where there have been deletions. Thus the fragment may comprise at most 5, 10, 20, 30, 40 or 50%, typically up to 60%, more typically up to 70%, preferably up to 80%, more preferably up to 90%, even more preferably up to 95%, yet more preferably up to 99% of the complete sequence of the full mature PSE1 protein. Particularly preferred fragments of PSE1 protein comprise one or more whole domains of the desired protein.

A fragment or variant of PSE1 may be a protein that, when expressed recombinantly in a host cell, such as S. cerevisiae, can complement the deletion of the endogenous PSE1 gene in the host cell and may, for example, be a naturally occurring homolog of PSE1, such as a homolog encoded by another organism, such as another yeast or other fungi, or another eukaryote such as a human or other vertebrate, or animal or by a plant.

Another preferred chaperone is ORM2 or a fragment or variant thereof having equivalent chaperone-like activity.

ORM2, also known as YLR350W, is located on chromosome XII (positions 828729 to 829379) of the S. cerevisiae genome and encodes an evolutionarily conserved protein with similarity to the yeast protein Orm1p. Hjelmqvist et al, 2002, Genome Biology, 3(6), research 0027.1-0027.16 reports that ORM2 belongs to gene family comprising three human genes (ORMDL1, ORMDL2 and ORMDL3) as well as homologs in microsporidia, plants, Drosophila, urochordates and vertebrates. The ORMDL genes are reported to encode transmembrane proteins anchored in the proteins endoplasmic reticulum (ER).

The protein Orm2p is required for resistance to agents that induce the unfolded protein response. Hjelmqvist et al, 2002 (supra) reported that a double knockout of the two S. cerevisiae ORMDL homologs (ORM1 and ORM2) leads to a decreased growth rate and greater sensitivity to tunicamycin and dithiothreitol.

One published sequence of Orm2p is as follows:

MIDRTKNESPAFEESPLTPNVSNLKPFPSQSNKISTPVTDHRRRRSSSVI
SHVEQETFEDENDQQMLPNMNATWVDQRGAWLIHIVVIVLLRLFYSLFGS
TPKWTWTLTNMTYIIGFYIMFHLVKGTPFDFNGGAYDNLTMWEQINDETL
YTPTRKFLLIVPIVLFLINQYYRNDMTLFLSNLAVTVLIGVVPKLGITHR
LRISIPGITGRAQIS*

The above protein is encoded in S. cerevisiae by the following coding nucleotide sequence, although it will be appreciated that the sequence can be modified by degenerate substitutions to obtain alternative nucleotide sequences which encode an identical protein product:

ATGATTGACCGCACTAAAAACGAATCTCCAGCTTTTGAAGAGTCTCCGCT
TACCCCCAATGTGTCTAACCTGAAACCATTCCCTTCTCAAAGCAACAAAA
TATCCACTCCAGTGACCGACCATAGGAGAAGACGGTCATCCAGCGTAATA
TCACATGTGGAACAGGAAACCTTCGAAGACGAAAATGACCAGCAGATGCT
TCCCAACATGAACGCTACGTGGGTCGACCAGCGAGGCGCGTGGTTGATTC
ATATCGTCGTAATAGTACTCTTGAGGCTCTTCTACTCCTTGTTCGGGTCG
ACGCCCAAATGGACGTGGACTTTAACAAACATGACCTACATCATCGGATT
CTATATCATGTTCCACCTTGTCAAGGTACGCCCTTCGACTTTAACGGTGG
TGCGTA0CGACAACCTGACCATGTGGGAGCAGATTAACGATGAGACTTTG
TACACACCCACTAGAAAATTTCTGCTGATTGTACCCATTGTGTTGTTCCT
GATTAGCAACCACTACTACCGCACGACATGACACTATTCCTCTCCAACCT
CGCCGTGACGGTGCTTATTGGTGTCGTTCCTAAGCTGGGAATTACGCATA
GACTAAGAATATCCATCCCTGGTATTACGGGCCGTGCTCAATTAGTTAG

Variants and fragments of ORM2 are also included in the present invention. A “variant”, in the context of ORM2, refers to a protein having the sequence of native ORM2 other than for at one or more positions where there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, He, Leu; Asp, Glu; Asn, Gln; Ser, Thr; Lys, Arg; and Phe, Tyr.

A “variant” of ORM2 typically has at least 25%, at least 50%, at least 60% or at least 70%, preferably at least 80%, more preferably at least 90%, even more preferably at least 95%, yet more preferably at least 99%, most preferably at least 99.5% sequence identity to the sequence of native ORM2.

The percent sequence identity between two polypeptides may be determined using suitable computer programs, as discussed below. Such variants may be natural or made using the methods of protein engineering and site-directed mutagenesis as are well known in the art.

A “fragment”, in the context of ORM2, refers to a protein having the sequence of native ORM2 other than for at one or more positions where there have been deletions. Thus the fragment may comprise at most 5, 10, 20, 30, 40 or 50%, typically up to 60%, more typically up to 70%, preferably up to 80%, more preferably up to 90%, even more preferably up to 95%, yet more preferably up to 99% of the complete sequence of the full mature ORM2 protein. Particularly preferred fragments of ORM2 protein comprise one or more whole domains of the desired protein.

A fragment or variant of ORM2 may be a protein that, when expressed recombinantly in a host cell, such as S. cerevisiae, can complement the deletion of the endogenous ORM2 gene in the host cell and may, for example, be a naturally occurring homolog of ORM2, such as a homolog encoded by another organism, such as another yeast or other fungi, or another eukaryote such as a human or other vertebrate, or animal or by a plant.

A gene encoding a protein comprising the sequence of a chaperone may be formed in a like manner to that discussed below for genes encoding heterologous proteins, with particular emphasis on combinations of ORFs and regulatory regions.

The term “protein” as used herein includes all natural and non-natural proteins, polypeptides and peptides. A “heterologous protein” is a protein that is not naturally encoded by a 2 μm-family plasmid and can also be described as a “non 2 μm-family plasmid protein”. For convenience, the terms “heterologous protein” and “non 2 μm-family plasmid protein” are used synonymously throughout this application. Preferably, therefore, the heterologous protein is not a FLP, REP1, REP2, or a RAF/D protein as encoded by any one of pSR1, pSB3 or pSB4 as, obtained from Z. rouxii, pSB1 or pSB2 both as obtained from Z. bailli, pSM1 as obtained from Z. fermentati, pKD1 as obtained from K. drosophilarum, pPM1 as obtained from P. membranaefaciens or the 2 μm plasmid as obtained from S. cerevisiae.

A gene encoding a heterologous protein comprises polynucleotide sequence encoding the heterologous protein (typically according to standard codon usage for any given organism), designated the open reading frame (“ORF”). The gene may additionally comprise some polynucleotide sequence that does not encode an open reading frame (termed “non-coding region”).

Non-coding region in the gene may contain one or more regulatory sequences, operatively linked to the ORF, which allow for the transcription of the open reading frame and/or translation of the resultant transcript.

The term “regulatory sequence” refers to a sequence that modulates (i.e., promotes or reduces) the expression (i.e., the transcription and/or translation) of an ORF to which it is operably linked. Regulatory regions typically include promoters, terminators, ribosome binding sites and the like. The skilled person will appreciate that the choice of regulatory region will depend upon the intended expression system. For example, promoters may be constitutive or inducible and may be cell- or tissue-type specific or non-specific.

Suitable regulatory regions, may be 5 bp, 10 bp, 15 bp, 20 bp, 25 bp, 30 bp, 35 bp, 40 bp, 45 bp, 50 bp, 60 bp, 70 bp, 80 bp, 90 bp, 100 bp, 120 bp, 140 bp, 160 bp, 180 bp, 200 bp, 220 bp, 240 bp, 260 bp, 280 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1100 bp, 1200 bp, 1300 bp, 1400 bp, 1500 bp or greater, in length.

Those skilled in the art will recognise that the gene encoding the chaperone, for example PDI, may additionally comprise non-coding regions and/or regulatory regions. Such non-coding regions and regulatory regions are not restricted to the native non-coding regions and/or regulatory regions normally associated with the chaperone ORF.

Where the expression system is yeast, such as Saccharomyces cerevisiae, suitable promoters for S. cerevisiae include those associated with the PGK1 gene, GAL1 or GAL10 genes, TEF1, TEF2, PYK1, PMA1, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucolinase, α-mating factor pheromone, a-mating factor pheromone, the PRB1 promoter, the PRA1 promoter, the GPD1 promoter, and hybrid promoters involving hybrids of parts of 5′ regulatory regions with parts of 5′ regulatory regions of other promoters or with upstream activation sites (e.g. the promoter of EP-A-258 067).

Suitable transcription termination signals are well known in the art. Where the host cell is eukaryotic, the transcription termination signal is preferably derived from the 3′ flanking sequence of a eukaryotic gene, which contains proper signals for transcription termination and polyadenylation. Suitable 3′ flanking sequences may, for example, be those of the gene naturally linked to the expression control sequence used, i.e. may correspond to the promoter. Alternatively, they may be different. In that case, and where the host is a yeast, preferably S. cerevisiae, then the termination signal of the S. cerevisiae ADH1, ADH2, CYC1, or PGK1 genes are preferred.

It may be beneficial for the promoter and open reading frame of the heterologous gene, such as the those of the chaperone PDI1, to be flanked by transcription termination sequences so that the transcription termination sequences are located both upstream and downstream of the promoter and open reading frame, in order to prevent transcriptional read-through into neighbouring genes, such as 2 μm genes, and visa versa.

In one embodiment, the favoured regulatory sequences in yeast, such as Saccharomyces cerevisiae, include: a yeast promoter (e.g. the Saccharomyces cerevisiae PRB1 promoter), as taught in EP 431 880; and a transcription terminator, preferably the terminator from Saccharomyces ADH1, as taught in EP 60 057. Preferably, the vector incorporates at least two translation stop codons.

It may be beneficial for the non-coding region to incorporate more than one DNA sequence encoding a translational stop codon, such as UAA, UAG or UGA, in order to minimise translational read-through and thus avoid the production of elongated, non-natural fusion proteins. The translation stop codon UAA is preferred.

The term “operably linked” includes within its meaning that a regulatory sequence is positioned within any non-coding region in a gene such that it forms a relationship with an ORF that permits the regulatory region to exert an effect on the ORF in its intended manner. Thus a regulatory region “operably linked” to an ORF is positioned in such a way that the regulatory region is able to influence transcription and/or translation of the ORF in the intended manner, under conditions compatible with the regulatory sequence.

In one preferred embodiment, the heterologous protein is secreted. In that case, a sequence encoding a secretion leader sequence which, for example, comprises most of the natural HSA secretion leader, plus a small portion of the S. cerevisiae α-mating factor secretion leader as taught in WO 90/01063 may be included in the open reading frame.

Alternatively, the heterologous protein may be intracellular.

In another preferred embodiment, the heterologous protein comprises the sequence of a eukaryotic protein, or a fragment or variant thereof. Suitable eukaryotes include fingi, plants and animals. In one preferred embodiment the heterologous protein is a fungal protein, such as a yeast protein. In another preferred embodiment the heterologous protein is an animal protein. Exemplary animals include vertebrates and invertebrates. Exemplary vertebrates include mammals, such as humans, and non-human mammals.

Thus the heterologous protein may comprise the sequence of a yeast protein. It may, for example, comprise the sequence of a yeast protein from the same host from which the 2 μm-family plasmid is derived. Those skilled in the art will recognise that a method, use or plasmid of the first, second or third aspects of the invention may comprise DNA sequences encoding more than one heterologous protein, more than one chaperone, or more than one heterologous protein and more than one chaperone.

In another preferred embodiment, the heterologous protein may comprise the sequence of albumin, a monoclonal antibody, an etoposide, a serum protein (such as a blood clotting factor), antistasin, a tick anticoagulant peptide, transferrin, lactoferrin, endostatin, angiostatin, collagens, immunoglobulins or immunoglobulin-based molecules or fragment of either (e.g. a Small Modular ImmunoPharmaceutical™ (“SMIP”) or dAb, Fab′ fragments, F(ab′)2, scAb, scFv or scFv fragment), a Kunitz domain protein (such as those described in WO 03/066824, with or without albumin fusions), interferons, interleukins, IL10, IL11, IL2, interferon α species and sub-species, interferon β species and sub-species, interferon γ species and sub-species, leptin, CNTF, CNTF_{Ax15}, IL1-receptor antagonist, erythropoietin (EPO) and EPO mimics, thrombopoietin (TPO) and TPO mimics, prosaptide, cyanovirin-N, 5-helix, T20 peptide, T1249 peptide, HIV gp41, HIV gp120, urokinase, prourokinase, tPA, hirudin, platelet derived growth factor, parathyroid hormone, proinsulin, insulin, glucagon, glucagon-like peptides, insulin-like growth factor, calcitonin, growth hormone, transforming growth factor β, tumour necrosis factor, G-CSF, GM-CSF, M-CSF, FGF, coagulation factors in both pre and active forms, including but not limited to plasminogen, fibrinogen, thrombin, pre-thrombin, pro-thrombin, von Willebrand's factor, α₁-antitrypsin, plasminogen activators, Factor VII, Factor VIII, Factor IX, Factor X and Factor XIII, nerve growth factor, LACI, platelet-derived endothelial cell growth factor (PD-ECGF), glucose oxidase, serum cholinesterase, aprotinin, amyloid precursor protein, inter-alpha trypsin inhibitor, antithrombin III, apo-lipoprotein species, Protein C, Protein S, or a variant or fragment of any of the above.

A “variant”, in the context of the above-listed proteins, refers to a protein wherein at one or more positions there have been amino acid insertions, deletions, or substitutions, either conservative or non-conservative, provided that such changes result in a protein whose basic properties, for example enzymatic activity or receptor binding (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” typically has at least 25%, at least 50%, at least 60% or at least 70%, preferably at least 80%, more preferably at least 90%, even more preferably at least 95%, yet more preferably at least 99%, most preferably at least 99.5% sequence identity to the polypeptide from which it is derived.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res., 22(22), 4673-80). The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Such variants may be natural or made using the methods of protein engineering and site-directed mutagenesis as are well known in the art.

A “fragment”, in the context of the above-listed proteins, refers to a protein wherein at one or more positions there have been deletions. Thus the fragment may comprise at most 5, 10, 20, 30, 40 or 50% of the complete sequence of the full mature polypeptide. Typically a fragment comprises up to 60%, more typically up to 70%, preferably up to 80%, more preferably up to 90%, even more preferably up to 95%, yet more preferably up to 99% of the complete sequence of the full desired protein. Particularly preferred fragments of a protein comprise one or more whole domains of the protein.

In one particularly preferred embodiment the heterologous protein comprises the sequence of albumin or a variant or fragment thereof.

By “albumin” we include a protein comprising the sequence of an albumin protein obtained from any source. Typically the source is mammalian. In one preferred embodiment the serum albumin is human serum albumin (“HSA”). The term “human serum albumin” includes the meaning of a serum albumin having an amino acid sequence naturally occurring in humans, and variants thereof. Preferably the albumin has the amino acid sequence disclosed in WO 90/13653 or a variant thereof. The HSA coding sequence is obtainable by known methods for isolating cDNA corresponding to human genes, and is also disclosed in, for example, EP 73 646 and EP 286 424.

In another preferred embodiment the “albumin” comprises the sequence of bovine serum albumin. The term “bovine serum albumin” includes the meaning of a serum albumin having an amino acid sequence naturally occurring in cows, for example as taken from Swissprot accession number P02769, and variants thereof as defined below. The term “bovine serum albumin” also includes the meaning of fragments of full-length bovine serum albumin or variants thereof, as defined below.

In another preferred embodiment the albumin comprises the sequence of an albumin derived from one of serum albumin from dog (e.g. see Swissprot accession number P49822), pig (e.g. see Swissprot accession number P08835), goat (e.g. as available from Sigma as product no. A2514 or A4164), turkey (e.g. see Swissprot accession number O73860), baboon (e.g. as available from Sigma as product no. A1516), cat (e.g. see Swissprot accession number P49064), chicken (e.g. see Swissprot accession number P19121), ovalbumin (e.g. chicken ovalbumin) (e.g. see Swissprot accession number P01012), donkey (e.g. see Swissprot accession number P39090), guinea pig (e.g. as available from Sigma as product no. A3060, A2639, O5483 or A6539), hamster (e.g. as available from Sigma as product no. A5409), horse (e.g. see Swissprot accession number P35747), rhesus monkey (e.g. see Swissprot accession number Q28522), mouse (e.g. see Swissprot accession number O89020), pigeon (e.g. as defined by Khan et al, 2002, Int. J. Biol. Macromol., 30(3-4), 171-8), rabbit (e.g. see Swissprot accession number P49065), rat (e.g. see Swissprot accession number P36953) and sheep (e.g. see Swissprot accession number P14639) and includes variants and fragments thereof as defined below.

Many naturally occurring mutant forms of albumin are known. Many are described in Peters, (1996, All About Albumin: Biochemistry, Genetics and Medical Applications, Academic Press, Inc., San Diego, Calif., p. 170-181). A variant as defined above may be one of these naturally occurring mutants.

A “variant albumin” refers to an albumin protein wherein at one or more positions there have been amino acid insertions, deletions, or substitutions, either conservative or non-conservative, provided that such changes result in an albumin protein for which at least one basic property, for example binding activity (type of and specific activity e.g. binding to bilirubin), osmolarity (oncotic pressure, colloid osmotic pressure), behaviour in a certain pH-range (pH-stability) has not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr. Such variants may be made by techniques well known in the art, such as by site-directed mutagenesis as disclosed in U.S. Pat. No. 4,302,386 issued 24 Nov. 1981 to Stevens, incorporated herein by reference.

Typically an albumin variant will have more than 40%, usually at least 50%, more typically at least 60%, preferably at least 70%, more preferably at least 80%, yet more preferably at least 90%, even more preferably at least 95%, most preferably at least 98% or more sequence identity with naturally occurring albumin. The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., 1994). The parameters used may be as follows:

Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

The term “fragment” as used above includes any fragment of full-length albumin or a variant thereof, so long as at least one basic property, for example binding activity (type of and specific activity e.g. binding to bilirubin), osmolarity (oncotic pressure, colloid osmotic pressure), behaviour in a certain pH-range (pH-stability) has not significantly been changed. “Significantly” in this context means that one skilled in the art would say that the properties of the variant may still be different but would not be unobvious over the ones of the original protein. A fragment will typically be at least 50 amino acids long. A fragment may comprise at least one whole sub-domain of albumin. Domains of HSA have been expressed as recombinant proteins (Dockal, M. et al., 1999, J. Biol. Chem., 274, 29303-29310), where domain I was defined as consisting of amino acids 1-197, domain II was defined as consisting of amino acids 189-385 and domain III was defined as consisting of amino acids 381-585. Partial overlap of the domains occurs because of the extended α-helix structure (h10-h1) which exists between domains I and II, and between domains II and III (Peters, 1996, op. cit., Table 2-4). HSA also comprises six sub-domains (sub-domains IA, IB, IIA, IIB, IIIA and IIIB). Sub-domain IA comprises amino acids 6-105, sub-domain IB comprises amino acids 120-177, sub-domain IIA comprises amino acids 200-291, sub-domain IIB comprises amino acids 316-369, sub-domain IIIA comprises amino acids 392-491 and sub-domain IIIB comprises amino acids 512-583. A fragment may comprise a whole or part of one or more domains or sub-domains as defined above, or any combination of those domains and/or sub-domains.

In another particularly preferred embodiment the heterologous protein comprises the sequence of transferrin or a variant or fragment thereof. The term “transferrin” as used herein includes all members of the transferrin family (Testa, Proteins of iron metabolism, CRC Press, 2002; Harris & Aisen, Iron carriers and iron proteins, Vol. 5, Physical Bioinorganic Chemistry, VCH, 1991) and their derivatives, such as transferrin, mutant transferrins (Mason et al, 1993, Biochemistry, 32, 5472; Mason et al, 1998, Biochem. J., 330(1), 35), truncated transferring, transferrin lobes (Mason et al, 1996, Protein Expr. Purif, 8, 119; Mason et al, 1991, Protein Expr. Purif., 2, 214), lactoferrin, mutant lactoferrins, truncated lactoferrins, lactoferrin lobes or fusions of any of the above to other peptides, polypeptides or proteins (Shin et al, 1995, Proc. Natl. Acad. Sci. USA, 92, 2820; Ali et al, 1999, J. Biol. Chem., 274, 24066; Mason et al, 2002, Biochemistry, 41, 9448).

The transferrin may be human transferrin. The term “human transferrin” is used herein to denote material which is indistinguishable from transferrin derived from a human or which is a variant or fragment thereof. A “variant” includes insertions, deletions and substitutions, either conservative or non-conservative, where such changes do not substantially alter the useful ligand-binding or immunogenic properties of transferrin.

Mutants of transferrin are included in the invention. Such mutants may have altered immunogenicity. For example, transferrin mutants may display modified (e.g. reduced) glycosylation. The N-linked glycosylation pattern of a transferrin molecule can be modified by adding/removing amino acid glycosylation consensus sequences such as N-X-S/T, at any or all of the N, X, or S/T position.

Transferrin mutants may be altered in their natural binding to metal ions and/or other proteins, such as transferrin receptor. An example of a transferrin mutant modified in this manner is exemplified below.

We also include naturally-occurring polymorphic variants of human transferrin or human transferrin analogues. Generally, variants or fragments of human transferrin will have at least 5%, 10%, 15%, 20%, 30%, 40% or 50% (preferably at least 80%, 90% or 95%) of human transferrin's ligand binding activity (for example iron-binding), weight for weight. The iron binding activity of transferrin or a test sample can be determined spectrophotometrically by 470 nm:280 nm absorbance ratios for the proteins in their iron-free and fully iron-loaded states. Reagents should be iron-free unless stated otherwise. Iron can be removed from transferrin or the test sample by dialysis against 0.1M citrate, 0.1M acetate, 10 mM EDTA pH4.5. Protein should be at approximately 20 mg/mL in 100 mM HEPES, 10 mM NaHCO₃ pH8.0. Measure the 470 nm:280 nm absorbance ratio of apo-transferrin (Calbiochem, CN Biosciences, Nottingham, UK) diluted in water so that absorbance at 280 nm can be accurately determined spectrophotometrically (0% iron binding). Prepare 20 mM iron-nitrilotriacetate (FeNTA) solution by dissolving 191 mg nitrotriacetic acid in 2 mL 1M NaOH, then add 2 mL 0.5M ferric chloride. Dilute to 50 mL with deionised water. Fully load apo-transferrin with iron (100% iron binding) by adding a sufficient excess of freshly prepared 20 mM FeNTA, then dialyse the holo-transferrin preparation completely against 100 mM HEPES, 10 mM NaHCO₃ pH8.0 to remove remaining FeNTA before measuring the absorbance ratio at 470 nm:280 nm. Repeat the procedure using test sample, which should initially be free from iron, and compare final ratios to the control.

Additionally, single or multiple heterologous fusions comprising any of the above; or single or multiple heterologous fusions to albumin, transferrin or immunoglobins or a variant or fragment of any of these may be used. Such fusions include albumin N-terminal fusions, albumin C-terminal fusions and co-N-terminal and C-terminal albumin fusions as exemplified by WO 01/79271, and transferrin N-terminal fusions, transferrin C-terminal fusions, and co-N-terminal and C-terminal transferrin fusions.

Examples of transferrin fusions are given in US patent applications US2003/0221201 and US2003/0226155, Shin, et al., 1995, Proc Natl Acad Sci USA, 92, 2820, Ali, et al., 1999, J Biol Chem, 274, 24066, Mason, et al., 2002, Biochemistry, 41, 9448, the contents of which are incorporated herein by reference.

The skilled person will also appreciate that the open reading frame of any other gene or variant, or part or either, can be utilised as an open reading frame for use with the present invention. For example, the open reading frame may encode a protein comprising any sequence, be it a natural protein (including a zymogen), or a variant, or a fragment (which may, for example, be a domain) of a natural protein; or a totally synthetic protein; or a single or multiple fusion of different proteins (natural or synthetic). Such proteins can be taken, but not exclusively, from the lists provided in WO 01/79258, WO 01/79271, WO 01/79442, WO 01/79443, WO 01/79444 and WO 01/79480, or a variant or fragment thereof; the disclosures of which are incorporated herein by reference. Although these patent applications present the list of proteins in the context of fusion partners for albumin, the present invention is not so limited and, for the purposes of the present invention, any of the proteins listed therein may be presented alone or as fusion partners for albumin, the Fc region of immunoglobulin, transferrin, lactoferrin or any other protein or fragment or variant of any of the above, as a desired polypeptide.

The heterologous protein may be a therapeutically active protein. In other words, it may have a recognised medical effect on individuals, such as humans. Many different types of therapeutically active protein are well known in the art.

The heterologous protein may comprise a leader sequence effective to cause secretion in yeast.

Numerous natural or artificial polypeptide signal sequences (also called secretion pre regions) have been used or developed for secreting proteins from host cells. The signal sequence directs the nascent protein towards the machinery of the cell that exports proteins from the cell into the surrounding medium or, in some cases, into the periplasmic space. The signal sequence is usually, although not necessarily, located at the N-terminus of the primary translation product and is generally, although not necessarily, cleaved off the protein during the secretion process, to yield the “mature” protein.

In the case of some proteins the entity that is initially secreted, after the removal of the signal sequence, includes additional amino acids at its N-terminus called a “pro” sequence, the intermediate entity being called a “pro-protein”. These pro sequences may assist the final protein to fold and become functional, and are usually then cleaved off. In other instances, the pro region simply provides a cleavage site for an enzyme to cleave off the pre-pro region and is not known to have another function.

The pro sequence can be removed either during the secretion of the protein from the cell or after export from the cell into the surrounding medium or periplasmic space.

Polypeptide sequences which direct the secretion of proteins, whether they resemble signal (i.e. pre) sequences or pre-pro secretion sequences, are referred to as leader sequences. The secretion of proteins is a dynamic process involving translation, translocation and post-translational processing, and one or more of these steps may not necessarily be completed before another is either initiated or completed.

For production of proteins in eukaryotic species such as the yeasts Saccharomyces cerevisiae, Zygosaccharomyces species, Kluyveromyces lactis and Pichia pastoris, known leader sequences include those from the S. cerevisiae acid phosphatase protein (Pho5p) (see EP 366 400), the invertase protein (Suc2p) (see Smith et al. (1985) Science, 229, 1219-1224) and heat-shock protein-150 (Hsp150p) (see WO 95/33833). Additionally, leader sequences from the S. cerevisiae mating factor alpha-1 protein (MFα-1) and from the human lysozyme and human serum albumin (HSA) protein have been used, the latter having been used especially, although not exclusively, for secreting human albumin. WO 90/01063 discloses a fusion of the MFα-1 and HSA leader sequences, which advantageously reduces the production of a contaminating fragment of human albumin relative to the use of the MFα-1 leader sequence. Modified leader sequences are also disclosed in the examples of this application and the reader will appreciate that those leader sequences can be used with proteins other than transferrin. In addition, the natural transferrin leader sequence may be used to direct secretion of transferrin and other heterologous proteins.

Where the chaperone is protein disulphide isomerase, then preferably the heterologous protein comprises disulphide bonds in its mature form. The disulphide bonds may be intramolecular and/or intermolecular.

The heterologous protein may be a commercially useful protein. Some heterologously expressed proteins are intended to interact with the cell in which they are expressed in order to bring about a beneficial effect on the cell's activities. These proteins are not, in their own right, commercially useful. Commercially useful proteins are proteins that have a utility ex vivo of the cell in which they are expressed. Nevertheless, the skilled reader will appreciate that a commercially useful protein may also have a biological effect on the host cell expressing it as a heterologous protein, but that that effect is not the main or sole reason for expressing the protein therein.

In one embodiment it is preferred that the heterologous protein is not β-lactamase. In another embodiment it is preferred that the heterologous protein is not antistasin. However, the reader will appreciate that neither of these provisos exclude genes encoding either β-lactamase or antistasin from being present on the 2 μm-family plasmid of the invention, merely that the gene encoding the heterologous protein encodes a protein other than β-lactamase and/or antistasin.

Plasmids can be prepared by modifying 2 μm-family plasmids known in the art by inserting a gene encoding a chaperone and inserting a gene encoding a heterologous protein using techniques well known in the art such as are described in by Sambrook et al., Molecular Cloning: A Laboratory Manual, 2001, 3rd edition, the contents of which are incorporated herein by reference. For example, one such method involves ligation via cohesive ends. Compatible cohesive ends can be generated on a DNA fragment for insertion and plasmid by the action of suitable restriction enzymes. These ends will rapidly anneal through complementary base pairing and remaining nicks can be closed by the action of DNA ligase.

A further method uses synthetic double stranded oligonucleotide linkers and adaptors. DNA fragments with blunt ends are generated by bacteriophage T4 DNA polymerase or E. coli DNA polymerase I which remove protruding 3′ termini and fill in recessed 3′ ends. Synthetic linkers and pieces of blunt-ended double-stranded DNA, which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end. Alternatively a DNA fragment or DNA fragments can be ligated together by the action of DNA ligase in the presence or absence of one or more synthetic double stranded oligonucleotides optionally containing cohesive ends.

Synthetic linkers containing a variety of restriction endonuclease sites are commercially available from a number of sources including Sigma-Genosys Ltd, London Road, Pampisford, Cambridge, United Kingdom.

Appropriate insertion sites in 2 μm-family plasmids include, but are not limited to, those discussed above.

The present invention also provides a host cell comprising a plasmid as defined above. The host cell may be any type of cell. Bacterial and yeast host cells are preferred. Bacterial host cells may be useful for cloning purposes. Yeast host cells may be useful for expression of genes present in the plasmid.

In one embodiment the host cell is a yeast cell, such as a member of the Saccharomyces, Kluyveromyces, or Pichia genus, such Saccharomyces cerevisiae, Kluyveromyces lactis, Pichia pastoris and Pichia membranaefaciens, or Zygosaccharomyces rouxii, Zygosaccharomyces bailii, Zygosaccharomyces fermentati, or Kluyveromyces drosphilarum are preferred.

The host cell type may be selected for compatibility with the plasmid type being used. Plasmids obtained from one yeast type can be maintained in other yeast types (Irie et al, 1991, Gene, 108(1), 139-144; Irie et al, 1991, Mol. Gen. Genet., 225(2), 257-265). For example, pSR1 from Zygosaccharomyces rouxii can be maintained in Saccharomyces cerevisiae. Preferably, the host cell is compatible with the 2 μm-family plasmid used (see below for a full description of the following plasmids). For example, where the plasmid is based on pSR1, pSB3 or pSB4 then a suitable yeast cell is Zygosaccharomyces rouxii; where the plasmid is based on pSB1 or pSB2 then a suitable yeast cell is Zygosaccharomyces bailli; where the plasmid is based on pSM1 then a suitable yeast cell is Zygosaccharomyces fermentati; where the plasmid is based on pKD1 then a suitable yeast cell is Kluyveromyces drosophilarum; where the plasmid is based on pPM1 then a suitable yeast cell is Pichia membranaefaciens; where the plasmid is based on the 2 μm plasmid then a suitable yeast cell is Saccharomyces cerevisiae or Saccharomyces carlsbergensis. It is particularly preferred that the plasmid is based on the 2 μm plasmid and the yeast cell is Saccharomyces cerevisiae.

A 2 μm-family plasmid of the invention can be said to be “based on” a naturally occurring plasmid if it comprises one, two or preferably three of the genes FLP, REP1 and REP2 having sequences derived from that naturally occurring plasmid.

It may be particularly advantageous to use a yeast deficient in one or more protein mannosyl transferases involved in O-glycosylation of proteins, for instance by disruption of the gene coding sequence.

Recombinantly expressed proteins can be subject to undesirable post-translational modifications by the producing host cell. For example, the albumin protein sequence does not contain any sites for N-linked glycosylation and has not been reported to be modified, in nature, by O-linked glycosylation. However, it has been found that recombinant human albumin (“rHA”) produced in a number of yeast species can be modified by O-linked glycosylation, generally involving mannose. The mannosylated albumin is able to bind to the lectin Concanavalin A. The amount of mannosylated albumin produced by the yeast can be reduced by using a yeast strain deficient in one or more of the PMT genes (WO 94/04687). The most convenient way of achieving this is to create a yeast which has a defect in its genome such that a reduced level of one of the Pmt proteins is produced. For example, there may be a deletion, insertion or transposition in the coding sequence or the regulatory regions (or in another gene regulating the expression of one of the PMT genes) such that little or no Pmt protein is produced.

Alternatively, the yeast could be transformed to produce an anti-Pmt agent, such as an anti-Pmt antibody.

If a yeast other than S. cerevisiae is used, disruption of one or more of the genes equivalent to the PMT genes of S. cerevisiae is also beneficial, e.g. in Pichia pastoris or Kluyveromyces lactis. The sequence of PMT1 (or any other PMT gene) isolated from S. cerevisiae may be used for the identification or disruption of genes encoding similar enzymatic activities in other fungal species. The cloning of the PMT1 homologue of Kluyveromyces lactis is described in WO 94/04687.

The yeast will advantageously have a deletion of the HSP150 and/or YAP3 genes as taught respectively in WO 95/33833 and WO 95/23857.

A plasmid as defined above, may be introduced into a host through standard techniques. With regard to transformation of prokaryotic host cells, see, for example, Cohen et al (1972) Proc. Natl. Acad. Sci. USA 69, 2110 and Sambrook et al (2001) Molecular Cloning, A Laboratory Manual, 3^{rd} Ed. Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. Methods for the transformation of S. cerevisiae are taught generally in EP 251 744, EP 258 067 and WO 90/01063, all of which are incorporated herein by reference. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming cells and is well known in the art for transforming yeast cell, bacterial cells and vertebrate cells. Methods for transformation of yeast by electroporation are disclosed in Becker & Guarente (1990) Methods Enzymol. 194, 182.

Generally, the plasmid will transform not all of the hosts and it will therefore be necessary to select for transformed host cells. Thus, a plasmid may comprise a selectable marker, including but not limited to bacterial selectable marker and/or a yeast selectable marker. A typical bacterial selectable marker is the β-lactamase gene although many others are known in the art. Typical yeast selectable marker include LEU2, TRP1, HIS3, HIS4, URA3, URA5, SFA1, ADE2, MET15, LYS5, LYS2, ILV2, FBA1, PSE1, PDI1 and PGK1. Those skilled in the art will appreciate that any gene whose chromosomal deletion or inactivation results in an inviable host, so called essential genes, can be used as a selective marker if a functional gene is provided on the plasmid, as demonstrated for PGK1 in a pgk1 yeast strain (Piper and Curran, 1990, Curr. Genet. 17, 119). Suitable essential genes can be found within the Stanford Genome Database (SGD), http:://db.yeastgenome.org). Any essential gene product (e.g. PDI1, PSE1, PGK1 or FBA1) which, when deleted or inactivated, does not result in an auxotrophic (biosynthetic) requirement, can be used as a selectable marker on a plasmid in a host cell that, in the absence of the plasmid, is unable to produce that gene product, to achieve increased plasmid stability without the disadvantage of requiring the cell to be cultured under specific selective conditions. By “auxotrophic (biosynthetic) requirement” we include a deficiency which can be complemented by additions or modifications to the growth medium. Therefore, preferred “essential marker genes” in the context of the present invention are those that, when deleted or inactivated in a host cell, result in a deficiency which cannot be complemented by additions or modifications to the growth medium.

Additionally, a plasmid according to any one of the first, second or third aspects of the present invention may comprise more than one selectable marker.

One selection technique involves incorporating into the expression vector a DNA sequence marker, with any necessary control elements, that codes for a selectable trait in the transformed cell. These markers include dihydrofolate reductase, G418 or neomycin resistance for eukaryotic cell culture, and tetracyclin, kanamycin or ampicillin (i.e. β-lactamase) resistance genes for culturing in E. coli and other bacteria. Alternatively, the gene for such selectable trait can be on another vector, which is used to co-transform the desired host cell.

Another method of identifying successfully transformed cells involves growing the cells resulting from the introduction of a plasmid of the invention, optionally to allow the expression of a recombinant polypeptide (i.e. a polypeptide which is encoded by a polynucleotide sequence on the plasmid and is heterologous to the host cell, in the sense that that polypeptide is not naturally produced by the host). Cells can be harvested and lysed and their DNA or RNA content examined for the presence of the recombinant sequence using a method such as that described by Southern (1975) J. Mol. Biol. 98, 503 or Berent et al (1985) Biotech. 3, 208 or other methods of DNA and RNA analysis common in the art. Alternatively, the presence of a polypeptide in the supernatant of a culture of a transformed cell can be detected using antibodies.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium. Alternatively, transformed cells may represent an industrially/commercially or pharmaceutically useful product and can be used without further purification or can be purified from a culture medium and optionally formulated with a carrier or diluent in a manner appropriate to their intended industrial/commercial or pharmaceutical use, and optionally packaged and presented in a manner suitable for that use. For example, whole cells could be immobilised; or used to spray a cell culture directly on to/into a process, crop or other desired target. Similarly, whole cell, such as yeast cells can be used as capsules for a huge variety of applications, such as fragrances, flavours and pharmaceuticals.

Transformed host cells may be cultured for a sufficient time and under appropriate conditions known to those skilled in the art, and in view of the teachings disclosed herein, to permit the expression of the chaperone and heterologous protein encoded by the plasmid.

The culture medium may be non-selective or place a selective pressure on the maintenance of the plasmid.

The thus produced heterologous protein may be present intracellularly or, if secreted, in the culture medium and/or periplasmic space of the host cell.

The step of “purifying the thus expressed heterologous protein from the cultured host cell or the culture medium” optionally comprises cell immobilization, cell separation and/or cell breakage, but always comprises at least one other purification step different from the step or steps of cell immobilization, separation and/or breakage.

Cell immobilization techniques, such as encasing the cells using calcium alginate bead, are well known in the art. Similarly, cell separation techniques, such as centrifugation, filtration (e.g. cross-flow filtration, expanded bed chromatography and the like are well known in the art. Likewise, methods of cell breakage, including beadmilling, sonication, enzymatic exposure and the like are well known in the art.

The at least one other purification step may be any other step suitable for protein purification known in the art. For example purification techniques for the recovery of recombinantly expressed albumin have been disclosed in: WO 92/04367, removal of matrix-derived dye; EP 464 590, removal of yeast-derived colorants; EP 319 067, alkaline precipitation and subsequent application of the albumin to a lipophilic phase; and WO 96/37515, U.S. Pat. No. 5,728,553 and WO 00/44772, which describe complete purification processes; all of which are incorporated herein by reference.

Proteins other than albumin may be purified from the culture medium by any technique that has been found to be useful for purifying such proteins.

Suitable methods include ammonium sulphate or ethanol precipitation, acid or solvent extraction, anion or cation exchange chromatography, phosphocellulose chromatography, hydrophobic interaction chromatography, affinity chromatography, hydroxylapatite chromatography, lectin chromatography, concentration, dilution, pH adjustment, diafiltration, ultrafiltration, high performance liquid chromatography (“HPLC”), reverse phase HPLC, conductivity adjustment and the like.

In one embodiment, any one or more of the above mentioned techniques may be used to further purifying the thus isolated protein to a commercially or industrially acceptable level of purity. By commercially or industrially acceptable level of purity, we include the provision of the protein at a concentration of at least 0.01 g.L⁻¹, 0.02 g.L⁻¹, 0.03 g.L⁻¹, 0.04 g.L⁻¹, 0.05 g.L⁻¹, 0.06 g.L⁻¹, 0.07 g.L⁻¹, 0.08 g.L⁻¹, 0.09 g.L⁻¹, 0.1 g.L⁻¹, 0.2 g.L⁻¹, 0.3 g.L⁻¹, 0.4 g.L⁻¹, 0.5 g.L⁻¹, 0.6 g.L⁻¹, 0.7 g.L⁻¹, 0.8 g.L⁻¹, 0.9 g.L⁻¹, 1 g.L⁻¹, 2 g.L⁻¹, 3 g.L⁻¹, 4 g.L⁻¹, 5 g.L⁻¹, 6 g.L⁻¹, 7 g.L⁻¹, 8 g.L¹, 9 g.L⁻¹, 10 g.L⁻¹, 15 g.L⁻¹, 20 g.L⁻¹, 25 g.L⁻¹, 30 g.L⁻¹, 40 g.L⁻¹, 50 g.L⁻¹, 60 g.L⁻¹, 70 g.L⁻¹, 70 g.L⁻¹, 90 g.L⁻¹, 100 g.L⁻¹, 150 g.L⁻¹, 200 g.L⁻¹, 250 g.L⁻¹, 300 g.L⁻¹, 350 g.L⁻¹, 400 g.L⁻¹, 500 g.L⁻¹, 600 g.L⁻¹, 700 g.L⁻¹, 800 g.L⁻¹, 900 g.L⁻¹, 1000 g.L⁻¹, or more.

It is preferred that the heterologous protein is purified to achieve a pharmaceutically acceptable level of purity. A protein has a pharmaceutically acceptable level of purity is it is essentially pyrogen free and can be administered in a pharmaceutically efficacious amount without causing medical effects not associated with the activity of the protein.

The resulting heterologous protein may be used for any of its known utilities, which, in the case of albumin, include i.v. administration to patients to treat severe burns, shock and blood loss, supplementing culture media, and as an excipient in formulations of other proteins.

Although it is possible for a therapeutically useful heterologous protein obtained by a process of the of the invention to be administered alone, it is preferable to present it as a pharmaceutical formulation, together with one or more acceptable carriers or diluents. The carrier(s) or diluent(s) must be “acceptable” in the sense of being compatible with the desired protein and not deleterious to the recipients thereof. Typically, the carriers or diluents will be water or saline which will be sterile and pyrogen free.

Optionally the thus formulated protein will be presented in a unit dosage form, such as in the form of a tablet, capsule, injectable solution or the like.

A further embodiment of the present invention provides a host cell recombinantly encoding proteins comprising the sequences of PDI and transferrin-based proteins. By “transferrin-based protein” we mean transferrin or any other member of the transferrin family (e.g. lactoferrin), a variant or fragment thereof or a fusion protein comprising transferrin, a variant or fragment thereof, including the types described above. Thus the present invention also provides for the use of a recombinant PDI gene to increase the expression of a transferrin-based protein.

The PDI gene may be provided on a plasmid, such as a 2 μm-family plasmid as described above. Alternatively, the PDI gene may be chromosomally integrated. In a preferred embodiment, the PDI gene is chromosomally integrated at the locus of an endogenously encoded PDI gene, preferably without disrupting the expression of the endogenous PDI gene. In this context, “without disrupting the expression of the endogenous PDI gene” means that, although some decrease in the protein production from the endogenous PDI gene as a result of the integration may be acceptable (and preferably there is no decrease), the total level of PDI protein production in the modified host cell as a result of the combined effect of expression from the endogenous and integrated PDI genes is increased, relative to the level of PDI protein production by the host cell prior to the integration event.

The gene encoding the transferrin-based protein may be provided on a plasmid, such as a 2 μm-family plasmid as described above, or may be chromosomally integrated, such as at the locus of an endogenously encoded PDI gene, preferably without disrupting the expression of the endogenous PDI gene.

In one embodiment the PDI gene is chromosomally integrated and the gene encoding the transferrin-based protein is provided on a plasmid. In another embodiment, the PDI gene is provided on a plasmid and the gene encoding the transferrin-based protein is chromosomally integrated. In another embodiment both the PDI gene and the gene encoding the transferrin-based protein are chromosomally integrated. In another embodiment both the PDI gene and the gene encoding the transferrin-based protein are provided on a plasmid.

As discussed above, Bao et al, 2000, Yeast, 16, 329-341 reported that over-expression of the K. lactis PDI gene KlPDI1 was toxic to K. lactis cells. Against this background we have surprisingly found that, not only is it possible to over-express PDI and other chaperones without the detrimental effects reported in Bao et al, but that two different chaperones can be recombinantly over-expressed in the same cell and, rather than being toxic, can increase the expression of heterologous proteins to levels higher than the levels obtained by individual expression of either of the chaperones. This was not expected. On the contrary, in light of the teaching of Bao et al, one would think that over-expression of two chaperones would be even more toxic than the over-expression of one. Moreover, in light of the earlier findings of the present invention, it was expected that the increases in heterologous protein expression obtained by co-expression with a single chaperone would be at the maximum level possible for the cell system used. Therefore, it was particularly surprising to find that yet further increases in heterologous protein expression could be obtained by co-expression of two different chaperones with the heterologous protein.

Accordingly, as a fifth aspect of the present invention there is provided a method for producing heterologous protein comprising providing a host cell (such as defined above) comprising a first recombinant gene encoding a protein comprising the sequence of a first chaperone protein, a second recombinant gene encoding a protein comprising the sequence of a second chaperone protein and a third recombinant gene encoding a heterologous protein, wherein the first and second chaperones are different; culturing the host cell in a culture medium under conditions that allow the expression of the first, second and third genes; and optionally purifying the thus expressed heterologous protein from the cultured host cell or the culture medium; and further optionally, lyophilising the thus purified protein.

The method may further comprise the step of formulating the purified heterologous protein with a carrier or diluent and optionally presenting the thus formulated protein in a unit dosage form, in the manner discussed above.

The term “recombinant gene” includes nucleic acid sequences that operate independently as “stand alone” expressible sequences to produce an encoded protein or, in the alternative, nucleic acid sequences introduced that operate in combination with endogenous sequences (such as by integration into an endogenous sequence so as to produce a nucleic acid sequence that is different to the endogenous sequence) within the host to cause increased expression of a target protein.

The first and second chaperones may be a chaperone as discussed above, and are a combination of chaperones that, when co-expressed in the same host cell, provide an additive effect to the increase in expression of the heterologous protein. By “additive effect” we include the meaning that the level of expression of the heterologous protein in the host cell is higher when the first and second recombinant genes are simultaneously co-expressed with the third recombinant gene as compared to the same system wherein (i) the first recombinant gene is co-expressed with the third recombinant gene in the absence of the expression of the second recombinant gene and (ii) the second recombinant gene is co-expressed with the third recombinant gene in the absence of the expression of the first recombinant gene.

One preferred chaperone is protein disulphide isomerase. Another preferred chaperone is ORM2 or a fragment or variant thereof. In a particularly preferred embodiment, the first and second chaperones are protein disulphide isomerase and ORM2 or a fragment or variant thereof.

The first, second and third recombinant genes may each individually be present on a plasmid within the host cell (such as a 2 μm-family plasmid, as discussed above) or be chromosomally integrated within the genome of the host cell. It will be appreciated that any combination of plasmid and chromosomally integrated first, second and third recombinant genes may be used. For example, the first, second and third recombinant genes may each individually be present on a plasmid, and this may be either the same plasmid or different plasmids. Alternatively, the first recombinant gene may be present on a plasmid, and second and third recombinant genes may be chromosomally integrated within the genome of the host cell. Alternatively, the first and second recombinant genes may be present on a plasmid and the third recombinant gene may be chromosomally integrated within the genome of the host cell. Alternatively, the first and third recombinant genes may be present on a plasmid and the second recombinant gene may be chromosomally integrated within the genome of the host cell. Alternatively, the first and second recombinant gene may be chromosomally integrated within the genome of the host cell and the third recombinant gene may be present on a plasmid. Alternatively, the first, second and third recombinant genes may each individually be chromosomally integrated within the genome of the host cell.

Particularly preferred plasmids are those defined above in respect of earlier aspects of the present invention. Accordingly, the present invention also provides a plasmid as defined above wherein the plasmid comprises two different genes (the first and second recombinant genes) encoding different chaperones. In one preferred embodiment, the plasmid may further comprise a gene encoding a heterologous protein (the third recombinant gene), such as a heterologous protein as described above.

In a sixth aspect of the present invention there is provided a method for producing a heterologous protein, such as a heterologous protein as defined above for an earlier aspect of the present invention, comprising: providing a host cell comprising a first recombinant gene encoding the protein comprising the sequence of ORM2 or a variant thereof and a second recombinant gene encoding a heterologous protein; culturing the host cell in a culture medium under conditions that allow the expression of the first and second genes; and purifying the thus expressed heterologous protein from the cultured host cell or the culture medium; and optionally, lyophilising the thus purified protein; and optionally formulating the purified heterologous protein with a carrier or diluent; and optionally presenting the thus formulated protein in a unit dosage form.

In the manner discussed above, the host cell may further comprise a further recombinant gene encoding a protein comprising the sequence of an alternative chaperone to ORM2 or a variant thereof.

Either or both of the first and second recombinant genes may be expressed from a plasmid, and preferably from the same plasmid. A further recombinant gene encoding a protein comprising the sequence of an alternative chaperone to ORM2 or a variant thereof may also be expressed from a plasmid, preferably from the same plasmid as either or both of the first and second recombinant genes. The plasmid may be a 2 μm-family plasmid, such as the 2 μm plasmid.

The present invention also provides, in a seventh aspect, for the use of a nucleic acid sequence encoding the protein ORM2 or a variant thereof to increase the production, in a host cell, of a heterologous protein encoded by a recombinant gene in the host cell by co-expression of the nucleic acid sequence and the recombinant gene within the host cell. Either or both of the nucleic acid sequence and the recombinant gene encoding the heterologous protein may be expressed from a plasmid within the host cell, and preferably from the same plasmid. In the manner discussed above, the host cell may further comprise a recombinant gene encoding an alternative chaperone to ORM2 or a variant thereof, which may be located on a plasmid within the host cell, preferably on the same plasmid as either or both of the nucleic acid sequence and the recombinant gene encoding the heterologous protein. Suitable plasmids include a 2 μm-family plasmid, such as the 2 μm plasmid, as discussed above.

In an eighth aspect of the present invention there is also provided the use of a plasmid as an expression vector to increase the production of a heterologous protein by providing a recombinant gene encoding the heterologous protein and a gene encoding ORM2 or a variant thereof on the same plasmid. The plasmid may further comprise a gene encoding an alternative chaperone to ORM2 or a variant thereof in the manner discussed above. Suitable plasmids include a 2 μm-family plasmid, such as the 2 μm plasmid, as discussed above.

Accordingly, in a ninth aspect, the present invention also provides a plasmid, preferably an expression plasmid, comprising a first gene encoding the protein ORM2 or a variant or fragment thereof and a second gene encoding a heterologous protein, as discussed above. The plasmid may further comprise a third gene encoding an alternative chaperone to ORM2 or a variant thereof. In a preferred embodiment, the third gene encodes a protein comprising the sequence of protein disulphide isomerase.

We have also demonstrated that a plasmid-borne gene encoding a protein comprising the sequence of an “essential” chaperone, such as PDI, can be used to stably maintain the plasmid in a host cell that, in the absence of the plasmid, does not produce the chaperone, and simultaneously increase the expression of a heterologous protein encoded by a recombinant gene within the host cell. This system is advantageous because it allows the user to minimise the number of recombinant genes that need to be carried by a plasmid. For example, typical prior art plasmids carry marker genes (such as those as described above) that enable the plasmid to be stably maintained during host cell culturing process. Such marker genes need to be retained on the plasmid in addition to any further genes that are required to achieve a desired effect. However, the ability of plasmids to incorporate exogenous DNA sequences is limited and it is therefore advantageous to minimise the number of sequence insertions required to achieve a desired effect. Moreover, some marker genes (such as auxotrophic marker genes) require the culturing process to be conducted under specific conditions in order to obtain the effect of the marker gene. Such specific conditions may not be optimal for cell growth or protein production, or may require inefficient or unduly expensive growth systems to be used.

For the purpose of increasing heterologous gene expression, we have found that it is possible to use a gene that recombinantly encodes a protein comprising the sequence of an “essential” chaperone for the dual purpose of increasing the production of a heterologous protein in a host cell and in the role of a selectable marker on a plasmid, where the plasmid is present within a cell that, in the absence of the plasmid, is unable to produce the chaperone. This system has the advantage that it minimises the number of recombinant genes that need to be carried by the plasmid. The system also has the advantage that the host cell can be cultured under conditions that do not have to be adapted for any particular marker gene, without loosing plasmid stability. For example, host cells produced using this system can be culture in rich media, which may be more economical than the minimal media that is commonly used to give auxotrophic marker genes their effect.

Accordingly, in a tenth aspect, the present invention also provides a host cell comprising a plasmid, the plasmid comprising a gene that encodes an essential chaperone wherein, in the absence of the plasmid, the host cell is unable to produce the chaperone. Preferably, in the absence of the plasmid, the host cell is inviable. The host cell may further comprise a recombinant gene encoding a heterologous protein, such as those described above in respect of earlier aspects of the invention.

The present invention also provides, in a eleventh aspect, a plasmid comprising, as the sole selectable marker, a gene encoding an essential chaperone. The plasmid may further comprise a gene encoding a heterologous protein. The plasmid may be a 2 μm-family plasmid.

The present invention also provides, in a twelfth aspect, a method for producing a heterologous protein comprising the steps of: providing a host cell comprising a plasmid, the plasmid comprising a gene that encodes an essential chaperone wherein, in the absence of the plasmid, the host cell is unable to produce the chaperone and wherein the host cell further comprises a recombinant gene encoding a heterologous protein; culturing the host cell in a culture medium under conditions that allow the expression of the essential chaperone and the heterologous protein; and optionally purifying the thus expressed heterologous protein from the cultured host cell or the culture medium; and further optionally, lyophilising the thus purified protein.

The method may further comprise the step of formulating the purified heterologous protein with a carrier or diluent and optionally presenting the thus formulated protein in a unit dosage form, in the manner discussed above. In one preferred embodiment, the method involves culturing the host cell in non-selective media, such as a rich media.

We have surprising also found that different PDI genes have the ability to increase the expression of heterologous proteins by different amounts under particular culture conditions. In particular, as discussed in Example 8, we have shown that the SKQ2n PDI1 gene provides for higher heterologous protein expression than the S288c PDI1 gene, when the host cells are cultured in minimal media.

The sole difference between the encoded proteins of the SKQ2n PDI1 and S288c PDI1 genes is that SKQ2n comprises the additional amino acids EADAEAEA at positions 506-513 (positions as defined with reference to Genbank accession no. CAA38402, as given above).

The differences between the gene sequences used are shown in the sequence alignment given in FIG. 94 and can be summarised as follows—
The promoter of SKQ2n includes a run of fourteen “TA” repeats, whereas the promoter of S288c only has twelve “TA” repeats;
Ser41 is encoded by TCT in SKQ2n, but by TCC in S288c;
Glu44 is encoded by GAA in SKQ2n but by GAG in S298c;
Leu262 is encoded by TTG in SKQ2n but by TTA in S288c;
Asp514 is encoded by GAC in SKQ2n but the homologous Asp506 is encoded by GAT in S288c;
The terminator sequence of SKQ2n contains a run of 8 consecutive “A” bases, whereas the terminator sequence of S288c contains a run of 7 consecutive “A” bases and does not include an “A” base at the equivalent of position 1880 in the SKQ2n gene;
The terminator sequence of SKQ2n has a “C” at position 1919, whereas the terminator sequence of S288c has a “T” at the equivalent position.

It may be advantageous to include any or all of the above mentioned features of the SKQ2n gene in a PDI gene of choice, in order to achieve the observed increase in heterologous protein expression when the host cells are cultured in minimal media.

Accordingly, in a thirteenth aspect, there is also provided a nucleotide sequence encoding a protein disulphide isomerase, for use in increasing the expression of a heterologous protein in a host cell by expression of the nucleotide sequence within the host cell, which host cell is cultured in minimal media, wherein the nucleotide sequence encoding the protein disulphide isomerase is characterised in that it has at least one of the following characteristics—
the nucleotide sequence comprises a promoter having the sequence of a natural PDI promoter or a functional variant thereof and comprises a run of fourteen “TA” repeats; or
the encoded protein disulphide isomerase comprises the amino acids EADAEAEA or a conservatively substituted variant thereof, typically at positions 506-513 as defined with reference to Genbank accession no. CAA38402; or
residue Ser41 of the encoded protein disulphide isomerase is encoded by the codon TCT; or
residue Glu44 of the encoded protein disulphide isomerase is encoded by the codon GAA; or
residue Leu262 of the encoded protein disulphide isomerase is encoded by codon TTG; or
residue Asp514 of the encoded protein disulphide isomerase is encoded by codon GAC; or
the nucleotide sequence comprises a terminator sequence having the sequence of a natural PDI terminator or a functional variant thereof and either comprises a run of 8 consecutive “A” bases and/or the base “C” at position 1919 (as defined by reference to position 1919 of the natural SKQ2n terminator sequence).

The present invention also provides, in a fourteenth aspect, a method for producing a heterologous protein comprising the steps of: providing a host cell comprising a recombinant gene that encodes a protein disulphide isomerase and having the sequence of the above-defined nucleic acid sequence, the host cell further comprising a recombinant gene encoding a heterologous protein; culturing the host cell in a minimal culture medium under conditions that allow the expression of the protein disulphide isomerase and the heterologous protein; and optionally purifying the thus expressed heterologous protein from the cultured host cell or the culture medium; and further optionally, lyophilising the thus purified protein; and optionally further formulating the purified heterologous protein with a carrier or diluent; and optionally presenting the thus formulated protein in a unit dosage form, in the manner discussed above.

The genes encoding the PDI and heterologous protein can be provided in the manner described above in respect of other embodiments of the present invention.

We have also found that the effects of recombinantly-provided chaperones according to the other embodiments of the present invention can be modulated by modifying the promoters that control the expression levels of the chaperone(s). Surprisingly we have found that, in some cases, shorter promoters result in increased heterologous protein expression. Without being bound by theory we believe that this is because the expression of a recombinant chaperone in host cells that already express heterologous proteins at high levels can cause the cells to overload themselves with heterologously expressed protein, thereby achieving little or no overall increase in heterologous protein production. In those cases, it may be beneficial to provide recombinant chaperone genes with truncated promoters.

Accordingly, in a fifteenth aspect of the present invention there is provided a polynucleotide (such as a plasmid as defined above) comprising the sequence of a promoter operably connected to a coding sequence encoding a chaperone (such as those described above), for use in increasing the expression of a heterologous protein (such as those described above) in a host cell (such as those described above) by expression of the polynucleotide sequence within the host cell, wherein the promoter is characterised in that it achieves a modified, such as a higher or lower, level of expression of the chaperone than would be achieved if the coding sequence were to be operably connected to its naturally occurring promoter.

The present invention also provides, in a sixteenth aspect, a method for producing a heterologous protein comprising the steps of: providing a host cell comprising a recombinant gene that comprising the sequence of promoter operably connected to a coding sequence encoding a chaperone, the promoter being characterised in that it achieves a lower level of expression of the chaperone than would be achieved if the coding sequence were to be operably connected to its naturally occurring promoter, and the host cell further comprising a recombinant gene encoding a heterologous protein; culturing the host cell under conditions that allow the expression of the chaperone and the heterologous protein; and optionally purifying the thus expressed heterologous protein from the cultured host cell or the culture medium; and further optionally, lyophilising the thus purified protein; and optionally further formulating the purified heterologous protein with a carrier or diluent; and optionally presenting the thus formulated protein in a unit dosage form, in the manner discussed above.

As is apparent from the examples of the present application, the combination of recombinantly expressed PDI and transferrin-based proteins provides a surprisingly high level of transferrin expression. For example, transferrin expression in a system that includes a chromosomally encoded recombinant PDI gene provided a 2-fold increase (compared to a control in which there is no chromosomally encoded recombinant PDI gene). This increase was 5-times greater than an equivalent system comprising a recombinant gene encoding human albumin in place of the recombinant transferrin gene.

The host may be any cell type, such as a prokaryotic cell (e.g. bacterial cells such as E. coli) or a eukaryotic cell. Preferred eukaryotic cells include fungal cells, such as yeast cells, and mammalian cells. Exemplary yeast cells are discussed above. Exemplary mammalian cells include human cells.

Host cells as described above can be cultured to produce recombinant transferrin-based proteins. The thus produced transferrin-based proteins can be isolated from the culture and purified, preferably to a pharmaceutically acceptable level of purity, for example using techniques known in the art and/or as set out above. Purified transferrin-based proteins may be formulated with a pharmaceutically acceptable carrier or diluent and may be presented in unit dosage form.

The present invention will now be exemplified with reference to the following non-limiting examples and figures.

### BRIEF DESCRIPTION OF THE FIGURES

FIGS. 1, 2, 4, 6 to 15, 22, 25, 27 to 52, 57 to 71, 74, 75, 77 to 79, 81 to 83, 85 to 91, 95 and 96 show various plasmid maps.

FIG. 3 shows plasmid insertion sites.

FIG. 5 shows a restriction map of a DNA fragment containing the PDI coding sequence.

FIG. 16 shows the results of rocket immunoelectrophoresis (RIE) determination of increased recombinant transferrin (N413Q, N611Q) secretion with PDI1 over-expression. Cryopreserved yeast stocks were grown for 4-days in 10 mL BMMD shake flask cultures and supernatants were loaded at 5 μL per well. Goat polyclonal anti-transferrin (human) antiserum (Calbiochem) was used at 40 μL per rocket immunoelectrophoresis gel (50 mL). A=Control strain [pSAC35], duplicate flasks; B=Control strain [pDB2536], duplicate flasks; C=Control strain [pDB2711], neat to 40-fold aqueous dilutions; D=Control strain [pDB2931], duplicate flasks; E=Control strain [pDB2929], neat to 40-fold aqueous dilutions.

FIG. 17 shows the results of RIE analysis of recombinant transferrin (N413Q, N611Q) secretion with and without PDI1 over-expression. Cryopreserved yeast stocks were grown for 4-days in 10 mL BMMD shake flask cultures and supernatants were loaded at 5 μL per well. Duplicate loadings were made of supernatants from two individual cultures of each strain. Goat polyclonal anti-transferrin (human) antiserum (Calbiochem) was used at 40 μL per rocket immunoelectrophoresis gel (50 mL). A=Control strain [pSAC35]; B=Control strain [pDB2536]; C=Control strain [pDB2711]; D=Control strain [pDB2931]; E=Control strain [pDB2929].

FIG. 18 shows the results of SDS-PAGE analysis of recombinant transferrin secretion with and without PDI1 over-expression. BMMD shake flask cultures were grown for 4-days and 10 μL supernatant analysed on non-reducing SDS-PAGE (4-12% NuPAGE®, MOPS buffer, InVitrogen) with GelCode® Blue reagent (Pierce). SeeBlue Plus2 Markers (InVitrogen). 1=pDB2536; 2=pDB2536; 3=pDB2711; 4=pDB2711; 5=pDB2931; 6=pDB2931; 7=pDB2929; 8=pDB2929; 9=pSAC35 control.

FIG. 19 shows RIE analysis of recombinant transferrin secretion from S. cerevisiae strains with an additional integrated copy of PDI1. 5-day BMMD shake flask culture supernatants were loaded at 5 mL per well. Strains contained: 1) pSAC35 (negative control); 2) pDB2536 (recombinant non-glycosylated transferrin (N413Q, N611Q)) or 3) pDB2506 (same as plasmid pDB2536 but the transferrin ORF encodes transferrin without the N→Q mutations at positions 413 and 611, i.e. recombinant glycosylated transferrin). Each well contained a sample derived from an individual transformant. Standards were human plasma holo-transferrin (Calbiochem) at 100, 50, 20, 10, 5 and 2 mg.L⁻¹.

FIG. 20 shows RIE analysis of recombinant transferrin secretion from Strain A [pDB2536] and Strain A [pDB2506] grown in shake flask culture. 5-day BMMD or YEPD shake flask culture supernatants were loaded in duplicate at 51-L per well.

FIG. 21 shows SDS-PAGE analysis of recombinant transferrin secreted from Strain A [pDB2536] and Strain A [pDB2506] grown in shake flask culture. Cultures were grown for 5-days in BMMD and 30 mL supernatants analysed on SDS-PAGE (4-12% NuPAGE™, MOPS Buffer, InVitrogen) stained with GelCode, Blue Reagent (Pierce). 1) Strain A [pDB2536] transformant 1; 2) Strain A [pDB2536] transformant 2; 3) Strain A [pSAC35] control; 4) Strain A [pDB2506] transformant 1; 5) SeeBlue, Plus2 Protein Standards (approximate molecular weights only).

FIG. 23 shows RIE of recombinant transferrin secreted from S. cerevisiae strains with different PDI1 copy numbers. 3-day BMMD shake flask culture supernatants were loaded at 5 mL per well. Goat polyclonal anti-transferrin (human) antiserum (Calbiochem) was used at 30 mL per rocket immunoelectrophoresis gel (50 mL). (A) supernatant from S. cerevisiae control strain [pDB2711] or [pDB2712]; (B) supernatant from Strain A [pDB2536]; (C) supernatant from control strain [pDB2536].

FIG. 24 shows SDS-PAGE analysis of recombinant transferrin secreted from S. cerevisiae strains with different PDI1 copy numbers. 4-12% NuPAGE reducing gel run with MOPS buffer (InVitrogen) after loading with 30 mL of 3-day BMMD shake flask culture supernatant per lane; (lane 1) supernatant from control strain [pDB2536]; (lane 2) supernatant from Strain A [pDB2536]; (lanes 3-6) supernatant from control strain [pDB2711] or [pDB2712]; (lane 7) molecular weight markers (SeeBlue Plus2, InVitrogen).

FIG. 26 shows RIE of recombinant transferrin secreted from different S. cerevisiae strains with and without additional PDI1 gene co-expression. 10 mL YEPD shake flasks were inoculated with yeast and incubated for 4-days at 30° C. 5 μL culture supernatant loaded per well of a rocket immunoelectrophoresis gel. Plasma Tf standards concentrations are in μg/mL. 20 μL goat anti-Tf/50 mL agragose. Precipin was stained with Coomassie blue.

FIG. 53 shows RIE analysis of rHA expression in different S. cerevisiae strains when co-expressed with PDI1 genes having different length promoters. 10 mL YEPD shake flasks were inoculated with yeast and incubated for 4-days at 30° C. 4 μL culture supernatant loaded per well of a rocket immunoelectrophoresis gel. rHA standards concentrations are in μg/mL. 400 μL goat anti-HA (Sigma product A-1151 resuspended in 5 mL water)/50 mL agarose. Precipin was stained with Coomassie blue.

FIG. 54 shows RIE analysis of rHA expression in different S. cerevisiae strains when co-expressed with PDI1 genes having different length promoters. 10 mL YEPD shake flasks were inoculated with yeast and incubated for 4-days at 30° C. 4 μL culture supernatant loaded per well of a rocket immunoelectrophoresis gel. rHA standards concentrations are in μg/mL. 400 μL goat anti-HA (Sigma product A-1151 resuspended in 5 mL water)/50 mL agarose. Precipin was stained with Coomassie blue.

FIG. 55 shows RIE analysis of rTF expression, when co-expressed with different PDI1 constructs. 10 mL BMMD shake flasks were inoculated with yeast and incubated for 4-days at 30° C. 5 μL culture supernatant was loaded per well of a rocket immunoelectrophoresis gel containing 25 μL goat anti-Tf/50 mL. Plasma Tf standards concentrations are in μg/mL. Precipin was stained with Coomassie blue.

FIG. 56 shows RIE analysis of rTF expression, when co-expressed with different PDI1 constructs. 10 mL YEPD shake flasks were inoculated with yeast and incubated for 4-days at 30° C. 5 μL culture supernatant was loaded per well of a rocket immunoelectrophoresis gel containing 25 μL goat anti-Tf/50 mL. Plasma Tf standards concentrations are in μg/mL. Precipin was stained with Coomassie blue.

FIG. 72 shows RIE analysis of rHA fusion proteins with and without co-expressed recombinant PDI1. 10 mL BMMD shake flasks were inoculated with YBX7 transformed with albumin fusion expression plasmids and incubated for 4-days at 30° C. 4 μL culture supernatant loaded per well of a rocket immunoelectrophoresis gel. rHA standards concentrations are in μg/mL. 200 μL goat anti-HA (Sigma product A-1151 resuspended in 5 mL water)/50 mL agarose. Precipin was stained with Coomassie blue.

FIG. 73 shows SDS-PAGE analysis of recombinant albumin fusion secretion with and without PDI1 present on the expression plasmid. 10 mL BMMD shake flasks were inoculated with yeast and incubated for 4-days at 30° C., 200 rpm. 30 μL supernatant analysed on non-reducing SDS-PAGE (4-12% NuPAGE®, MES buffer, InVitrogen) with GelCode® Blue reagent (Pierce). 1=SeeBlue Plus2 Markers (InVitrogen); 2=1 μg rHA; 3=angiostatin-rHA; 4=angiostatin-rHA+PDI1; 5 endostatin-rHA; 6=endostatin-rHA+PDI1; 7=DX-890-(GGS)₄GG-rHA; 8=DX-890-(GGS)₄GG-rHA+PDI1; 9=DPI-14-(GGS)₄GG-rHA; 10=DPI-14-(GGS)₄GG-rH-A+PDI1; 11=Axokine™ (CNTF_{Ax15})-(GGS)₄GG-rHA (Lambert et al, 2001, Proc. Natl. Acad. Sci. USA, 98, 4652-4657); 12=Axokine™ (CNTF_{Ax15})-(GGS)₄GG-rHA+PDI1.

FIG. 76 shows RIE analysis demonstrating increased transferrin secretion from S. cerevisiae with ORM2 co-expression from a 2 μm-based plasmid. Four day shake flask culture supernatants were loaded at 5 μl per well. Standards were human plasma holo-transferrin (Calbiochem), at 25, 20, 15, 10, 5 μg/ml, loaded 5 μl per well. Goat polyclonal anti-transferrin (human) antiserum (Calbiochem) used at 20 μl per rocket immunoelectrophoresis gel (50 ml).

FIG. 80 shows RIE analysis demonstrating increased transferrin secretion from S. cerevisiae with PSE1 co-expression from a 2 μm-based plasmid. Four day shake flask culture supernatants were loaded at 5 μl per well. Standards were human plasma holo-transferrin (Calbiochem), at 25, 20, 15, 10, 5 μg/ml, loaded 5 μl per well. Goat polyclonal anti-transferrin (human) antiserum (Calbiochem) used at 20 μl per rocket immunoelectrophoresis gel (50 ml).

FIG. 84 shows RIE analysis demonstrating increased transferrin secretion from S. cerevisiae with SSA1 co-expression from a 2 nm-based plasmid. Four day shake flask culture supernatants were loaded at 5 μl per well. Standards were human plasma holo-transferrin (Calbiochem), at 25, 20, 15, 10, 5 μg/ml, loaded 5 μl per well. Goat polyclonal anti-transferrin (human) antiserum (Calbiochem) used at 20 μl per rocket immunoelectrophoresis gel (50 ml).

FIG. 92 shows the results of RIE. 10 mL YEPD shake flasks were inoculated with DXY1 trp1Δ [pDB2976], DXY1 trp1Δ [pDB2977], DXY1 trp1Δ [pDB2978], DXY1 trp1Δ [pDB2979], DXY1 trp1Δ [pDB2980] or DXY1 trp1Δ [pDB2981] transformed to tryptophan prototrophy with a 1.41 kb NotI/PstI pdi1::TTP1 disrupting DNA fragment was isolated from pDB3078. Transformants were grown for 4-days at 30° C., 200 rpm. 4 μL culture supernatant loaded per well of a rocket immunoelectrophoresis gel. rHA standards concentrations are in μg/mL. 700 μL goat anti-HA (Sigma product A-1151 resuspended in 5 mL water)/50 mL agarose. Precipin was stained with Coomassie blue. Isolates selected for further analysis are indicated (*).

FIG. 93 shows the results of RIE. 10 mL YEPD shake flasks were inoculated with DXY1 [pDB2244], DXY1 [pDB2976], DXY1 trp1Δ pdi1::TRP1 [pDB2976], DXY1 [pDB2978], DXY1 trp1Δ pdi1::TRP1 [pDB2978], DXY1 [pDB2980], DXY1 trp1Δ pdi1::TRP1 [pDB2980], DXY1 [pDB2977], DXY1 trp1Δ pdi1::TRP1 [pDB2977], DXY1 [pDB2979] DXY1 trp1Δ pdi1::TRP1 [pDB2979], DXY1 [pDB2981] and DXY1 trp1Δ pdi1::TRP1 [pDB2981], and were grown for 4-days at 30° C., 200 rpm. 4 μL culture supernatant loaded per well of a rocket immunoelectrophoresis gel. rHA standards concentrations are in μg/mL. 800 μL goat anti-HA (Sigma product A-1151 resuspended in 5 mL water)/50 mL agarose. Precipin was stained with Coomassie blue. Isolates selected for further analysis are indicated (*)

FIG. 94 shows a sequence alignment of the SKQ2n and S288c gene sequences with long promoters, as described in Example 6.

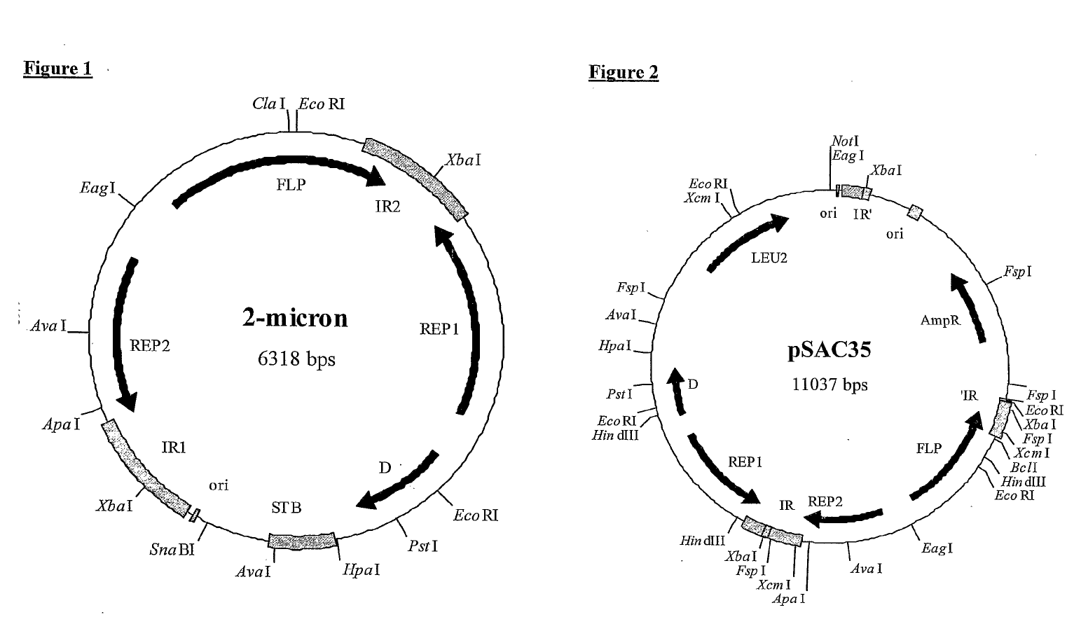

### EXAMPLES

Two types of expression cassette have been used to exemplify secretion of a recombinant human transferrin mutant (N413Q, N611Q) from S. cerevisiae. One type uses a modified HSA(pre)/MFα1(pro) leader sequence (named the “modified fusion leader” sequence). The second type of expression cassette uses only the modified HSA(pre) leader sequence.

The 24 amino acid sequence of the “modified fusion leader” is MKWVFIVSILFLFSSAYSRSLDKR.

The 18 amino acid sequence of the modified HSA(pre) leader sequence is MKWVFIVSILFLFSSAYS.

Transferrin (N413Q, N611Q) expression using these two cassettes has been studied in S. cerevisiae using the 2 μm expression vector with and without an additional copy of the S. cerevisiae PDI gene, PDI1.

### Example 1

### Construction of Expression Plasmids

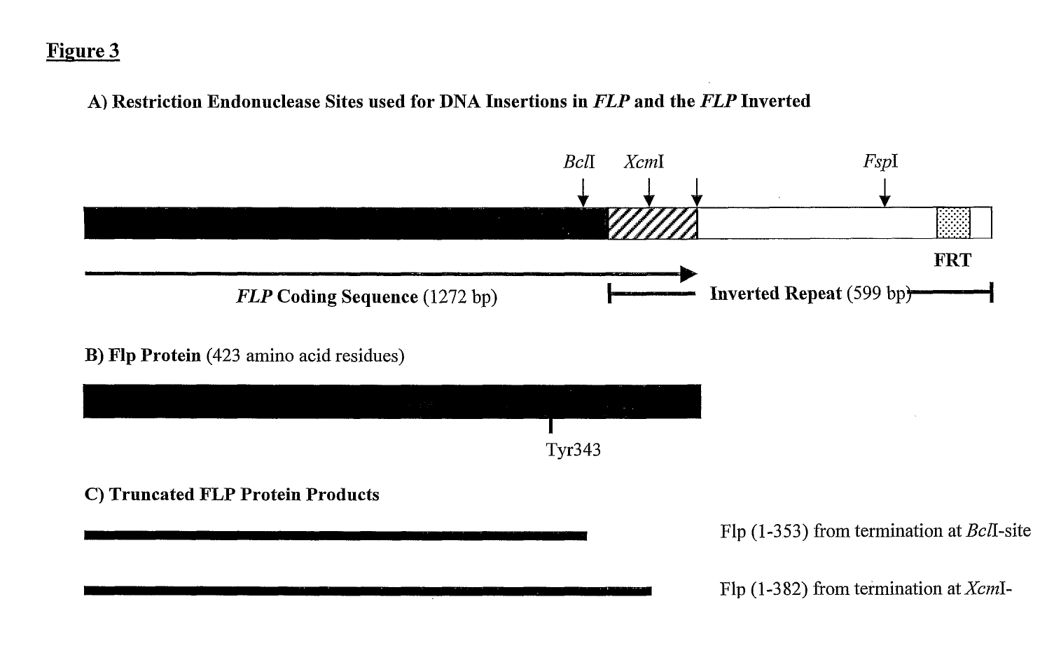

A 52-bp linker made by annealing 0.5 mM solutions of oligonucleotides CF86 and CF87 (see below) was introduced into the US-region of the 2 μm plasmid pSAC35 at the XcmI-sites in the 599-bp inverted repeats. One XcmI-site cuts 51-bp after the REP2 translation termination codon, whereas the other XcmI-site cuts 127-bp before the end of the FLP coding sequence, due to overlap with the inverted repeat (see FIG. 3). This DNA linker contained a core region “SnaBI-PacI-FseI/SfiI-SmaI-SnaBI”, which encoded restriction sites absent from pSAC35.

XcmI Linker (CF86 + CF87)

| | | | |
|---|---|---|---|
| | SfiI | | |
| | -------------- | | |
| | PacI | SnaBI | |
| | --------- ------- | | |
| | SnaBT | FseI | SmaI |
| | ------- -------- ------ | | |
| CF86 | GGAGTGGTA CGTATTAATT AAGGCCGGCC AGGCCCGGGT ACGTACCAAT TGA | | |
| CF87 | TCCTCACCAT GCATAATTAA TTCCGGCCGG TCCGGGCCA TGCATGGTTA AC | | |

Plasmid pSAC35 was partially digested with XcmI, the linear 11-kb fragment was isolated from a 0.7% (w/v) agarose gel, ligated with the CF86/CF87 XcmI linker (neat, 10⁻¹ and 10⁻² dilutions) and transformed into E. coli DH5α. Ampicillin resistant transformants were selected and screened for the presence of plasmids that could be linearised by SmaI digestion. Restriction enzyme analysis identified pDB2688 (FIG. 4) with the linker cloned into the XcmI-site after REP2. DNA sequencing using oligonucleotides primers CF88, CF98 and CF99 (Table 1) confirmed the insertion contained the correct linker sequence.

TABLE 1
Oligonucleotide sequencing primers:

| | | |
|---|---|---|
| Primer | Description | Sequence |

| | | |
|---|---|---|
| CF88 | REP2 primer, | 5′-ATCACGTAATACTTCTAGGG-3′ |
| | 20 mer | |
| CF98 | REP2 primer, | 5′-AGAGTGAGTTGGAAGGAAGG-3′ |
| | 20 mer | |
| CF99 | REP2 primer, | 5′-AGCTCGTAAGCGTCGTTACC-3′ |
| | 20 mer | |

The yeast strain was transformed to leucine prototrophy using a modified lithium acetate method (Sigma yeast transformation kit, YEAST-1, protocol 2; (Ito et al, 1983, J. Bacteriol., 153, 163; Elble, 1992, Biotechniques, 13, 18)). Transformants were selected on BMMD-agar plates, and were subsequently patched out on BMMD-agar plates. Cryopreserved trehalose stocks were prepared from 10 mL BMMD shake flask cultures (24 hrs, 30° C., 200 rpm), by addition of an equal volume of sterile 40% (w/v) trehalose

The composition of YEPD and BMMD is described by Sleep et al., 2002, Yeast, 18, 403. YEPS and BMMS are similar in composition to YEPD and BMMD accept that 2% (w/v) sucrose was substituted for the 2% (w/v) glucose as the sole initial carbon source.

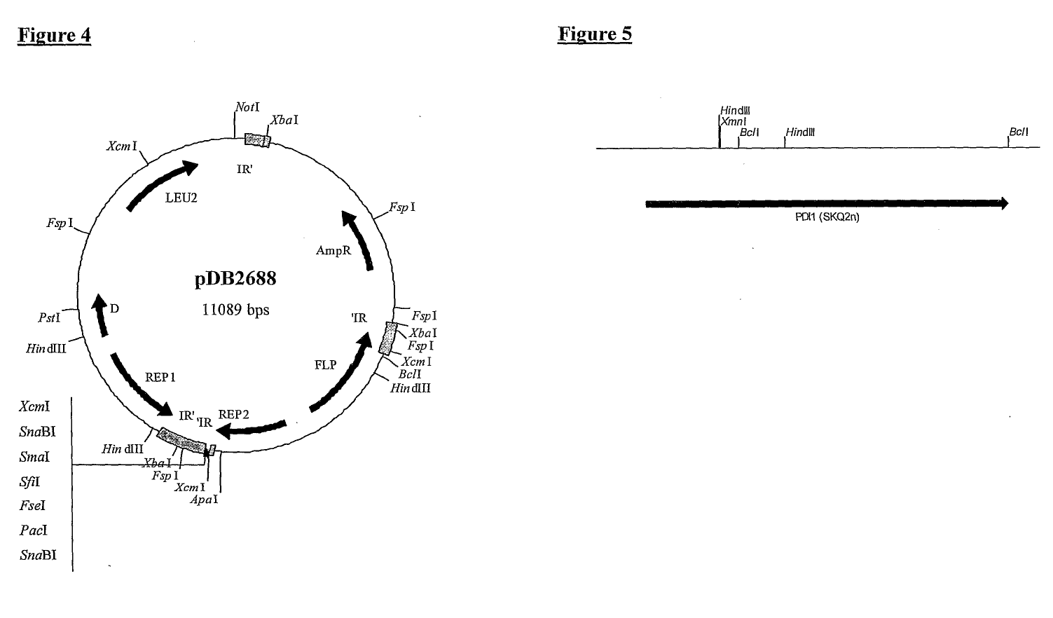

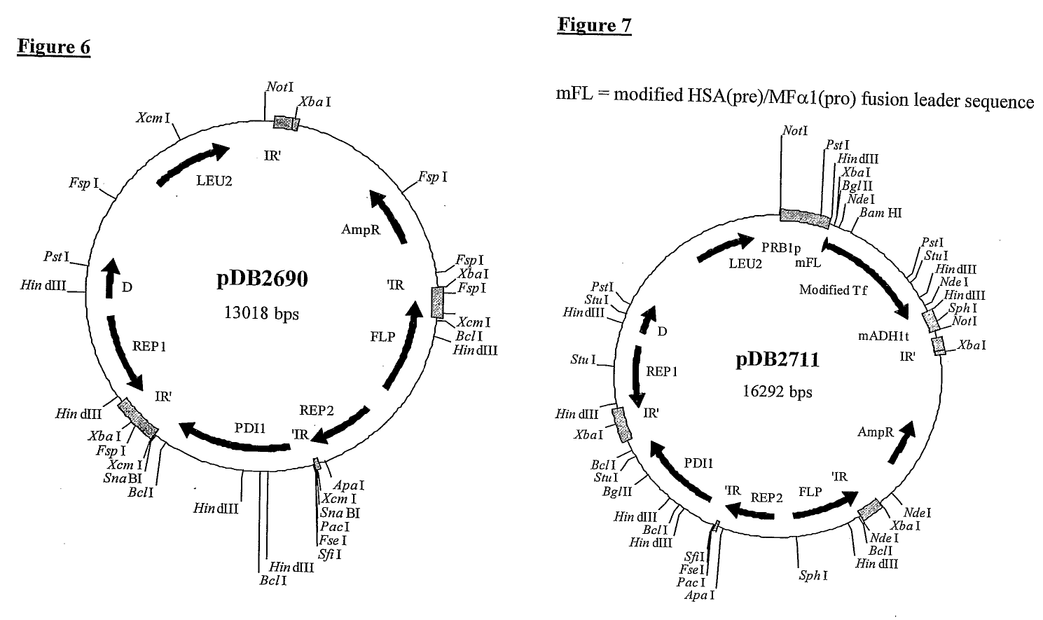

The S. cerevisiae PDI1 gene was cloned into the XcmI-linker of pDB2688. The PDI1 gene (FIG. 5) was cloned on a 1.9-kb SacI-SpeI fragment from a larger S. cerevisiae genomic SKQ2n DNA fragment containing the PDI1 gene (as provided in the plasmid pMA3a:C7 that is described in U.S. Pat. No. 6,291,205 and also described as Clone C7 in Crouzet & Tuite, 1987, Mol. Gen. Genet., 210, 581-583 and Farquhar et al, 1991, supra), which had been cloned into YIplac211 (Gietz & Sugino, 1988, Gene, 74, 527-534), and had a synthetic DNA linker containing a SacI restriction site inserted at a unique Bsu36I-site in the 3′ untranslated region of the PDI1 gene. The 1.9-kb SacI-SpeI fragment was treated with T4 DNA polymerase to fill the SpeI 5′-overhang and remove the SacI 3′-overhang. This PDI1 fragment included 212-bp of the PDI1 promoter upstream of the translation initiation codon, and 148-bp downstream of the translation termination codon. This was ligated with SmaI linearised/calf intestinal alkaline phosphatase treated pDB2688, to create plasmid pDB2690 (FIG. 6), with the PDI1 gene transcribed in the same direction as REP2. A S. cerevisiae strain was transformed to leucine prototrophy with pDB2690.

An expression cassette for a human transferrin mutant (N413Q, N611Q) was subsequently cloned into the NotI-site of pDB2690 to create pDB2711 (FIG. 7). The expression cassette in pDB2711 contains the S. cerevisiae PRB1 promoter, an HSA/MFα fusion leader sequence (EP 387319; Sleep et al, 1990, Biotechnology (N.Y.), 8, 42) followed by a coding sequence for the human transferrin mutant (N413Q, N611Q) and the S. cerevisiae ADH1 terminator. Plasmid pDB2536 was constructed similarly by insertion of the same expression cassette into the NotI-site of pSAC35.

The “modified fusion leader” sequence used in pDB2536 and pDB2711 comprises a modified HSA-pre sequence and a MFα1-pro sequence. An alternative leader sequence used was the modified HSA-pre sequence, which was derived from the modified fusion leader sequence by removal of the six residues of the MFα1-pro sequence.

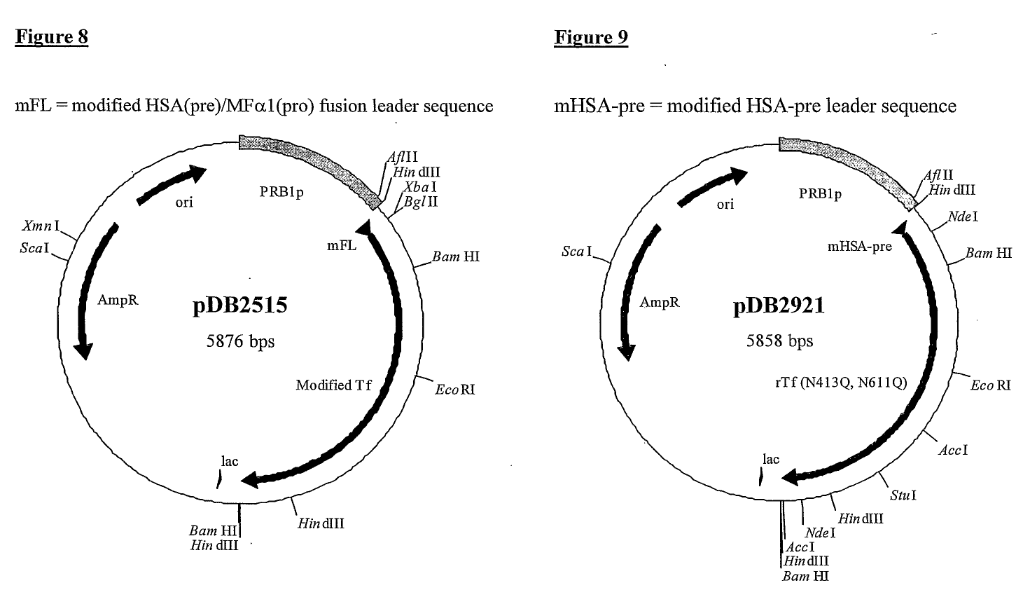

The modified fusion leader sequence in pDB2515 (FIG. 8) was mutated with oligonucleotides CF154 and CF155 to delete the coding sequence for the six residues (RSLDKR) of the MFα1-pro region. This was performed according to the instruction manual of the Statagene's QuickChange™ Site-Directed Mutagenesis Kit. pDB2515 is the E. coli cloning vector pGEM-7Z(−) (Promega) containing the 2940-bp NotI-HindIII (partial) DNA fragment of pDB2529 (see below) ligated between the PspOMI and HindIII sites.

CF154
5′-GTTCTTGTTCTCCTCTGCTTACTCTGTCCCTGATAAAACTGTGAGAT
GG-3′
CF155
5′-CCATCTCACAGTTTTATCAGGGACAGAGTAAGCAGAGGAGAACAAGA
AC-3′

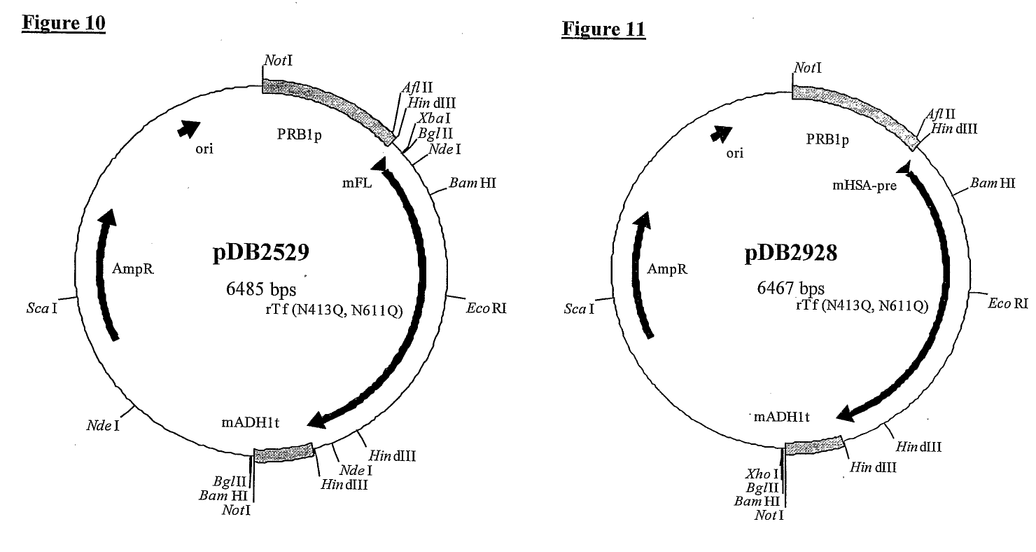

Competent E. coli DH5α cells were transformed with the mutated plasmids and ampicillin resistant colonies were selected. Plasmid DNA from these colonies was screened by double digestion with EcoRI and BglII. The correct DNA sequence for the modified HSA-pre leader was subsequently confirmed in pDB2921 (FIG. 9) over a 386-bp region between the AflII and BamHI sites either side of the leader sequence. This 386-bp AflII-BamHI fragment was isolated, and ligated with a 6,081-bp AflII-BamHI fragment from pDB2529 (FIG. 10), prepared by partial digestion with BamHI and complete digestion with AflII and calf intestinal alkaline phosphatase. pDB2529 is the E. coli cloning vector pBST(+) (Sleep et al, 2001, Yeast, 18, 403-441) containing the transferrin expression cassette of pDB2536 cloned into the unique NotI-site. This produced pDB2928 (FIG. 11), which was isolated from ampicillin resistant E. coli DH5α cells transformed with the ligation products.

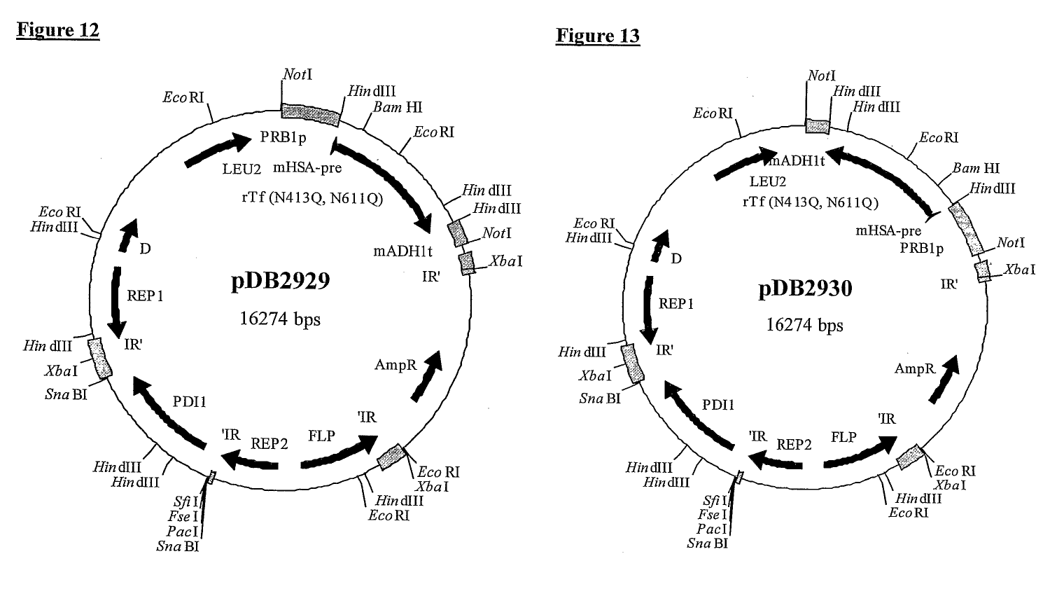

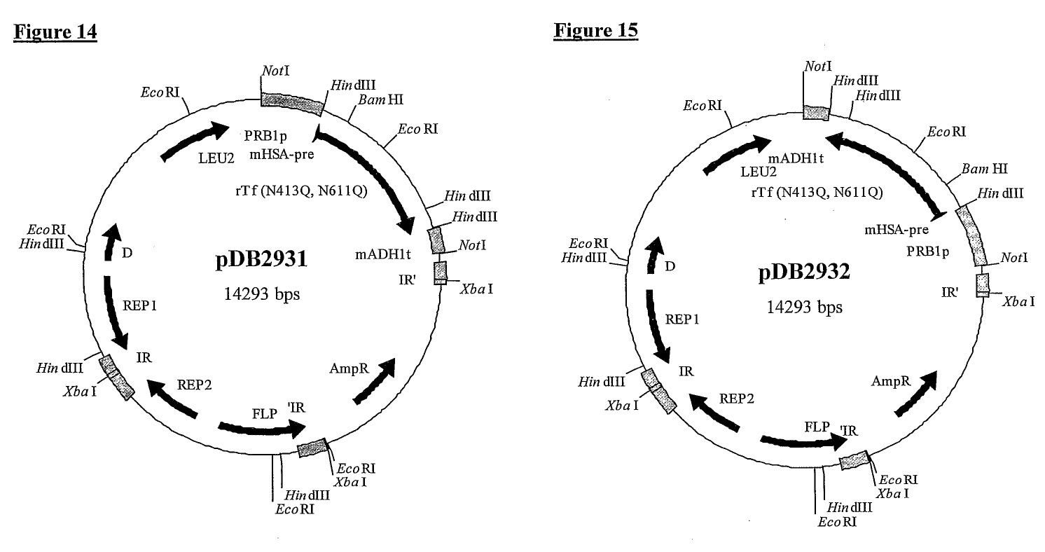

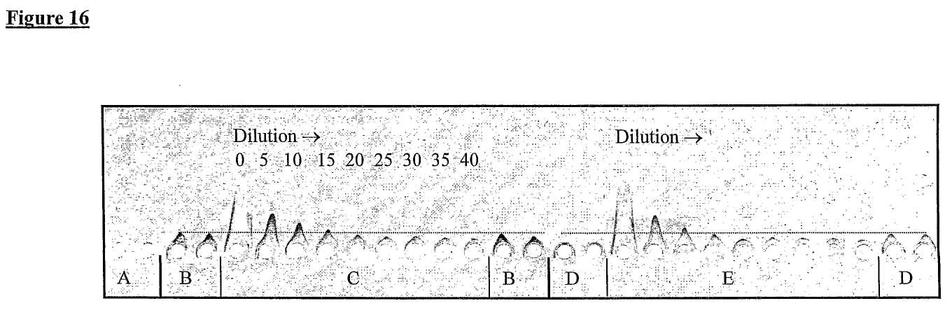

The 3,256-bp NotI expression cassette was isolated from pDB2928. This contained the PRB1 promoter, the coding region for the modified HSA-pre leader sequence followed by transferrin (N413Q, N611Q), and the ADH1 terminator. This was ligated into the NotI sites of the 2 μm-based vectors pSAC35 and pDB2690 to generate the expression plasmids pDB2929, pDB2930, pDB2931 and pDB2932 (FIGS. 12-15). In pDB2929 and pDB2931 the transferrin (N413Q, N611Q) sequence is transcribed in the same direction as LEU2, whereas in pDB2930 and pDB2932 transcription is in the opposite direction.

### Example 2

### Expression of Transferrin

A S. cerevisiae control strain was transformed to leucine prototrophy with all the transferrin (N413Q, N611Q) expression plasmids, and cryopreserved stocks were prepared.

Strains were grown for four days at 30° C. in 10 mL BMMD cultures in 50 mL conical flasks shaken at 200 rpm. The titres of recombinant transferrin secreted into the culture supernatants were compared by rocket immunoelectrophoresis (RIE as described in Weeke, B., 1976, “Rocket immunoelectrophoresis” In N. H. Azelsen, J. Kroll, and B. Weeke [eds.], A manual of quantitative immunoelectrophoresis. Methods and applications. Universitetsforlaget, Oslo, Norway), reverse phase high performance liquid chromatography (RP-HPLC) (Table 2), and non-reducing SDS polyacrylamide electrophoresis stained with colloidal Coomassie blue stain (SDS-PAGE). The increase in recombinant transferrin secreted when S. cerevisiae PDI1 was over-expressed was estimated to be greater than 10-fold.

TABLE 2

| | | Addi- | Average Transferrin | Estimated Increase |
|---|---|---|---|---|
| | Secretory | tional | Titre (μg · mL⁻¹) | due to Additional |
| Plasmid | Leader | PDI1 | (n = 2) | PDI1 |
| pSAC35 | None | No | 0.4 | — |
| pDB2536 | Fusion | No | 6.2 | — |
| | Leader | | | |
| pDB2711 | Fusion | Yes | 112.8 | 18-fold |
| | Leader | | | |
| pDB2931 | Modified | No | 5.1 | — |
| | HSA-pre | | | |
| | Leader | | | |
| pDB2929 | Modified | Yes | 76.1 | 15-fold |
| | HSA-pre | | | |
| | Leader | | | |

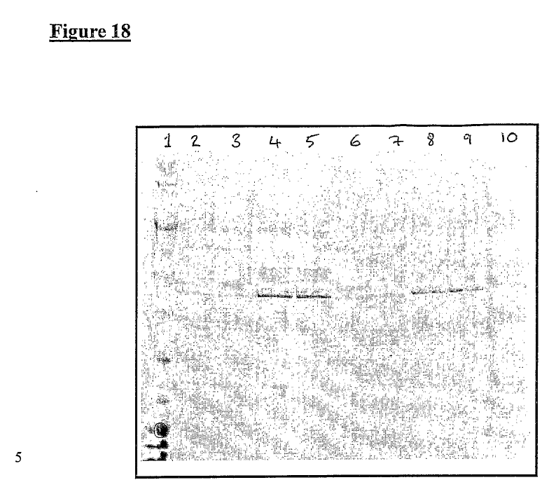

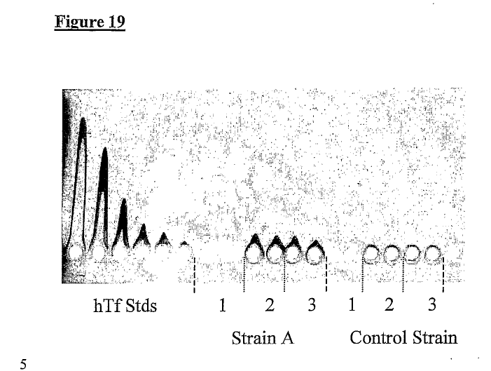

RIE analysis indicated that the increased transferrin secretion in the presence of additional copies of PDI1 was approximately 15-fold (FIG. 16). By RIE analysis the increase appeared slightly larger for the modified HSA-pre leader sequence than for the modified fusion leader sequence (FIG. 17).

By RP-HPLC analysis the increase in transferrin secretion was determined to be 18-fold for the modified fusion leader sequence and 15-fold for the modified HSA-pre leader sequence (Table 2).

FIG. 18 shows an SDS-PAGE comparison of the recombinant transferrin secreted by S. cerevisiae strains with and without additional PDI1 expression.

RP-HPLC Method for Determining Transferrin Expression

| | |
|---|---|
| Column: | 50 × 4.6 mm Phenomenex Jupiter C4 |
| | 300 Å, 5 μm |
| Column temperature: | 45° C. |
| Flow rate: | 1 mL · min⁻¹ |
| Peak detection: | UV absorbance at 214 nm |
| HPLC mobile phase A: | 0.1% TFA, 5% Acetonitrile |
| HPLC mobile phase B: | 0.1% TFA, 95% Acetonitrile |
| Gradient: | 0 to 3 minutes 30% B |
| | 3 to 13 minutes 30 to 55% B in a linear |
| | gradient |
| | 13 to 14 minutes 55% B |
| | 14 to 15 minutes 55 to 30% B in a linear |
| | gradient |
| | 15 to 20 minutes 30% B |
| Injection: | Generally 100 μL of sample, but any |
| | volume can be injected |
| Standard Curve: | 0.1 to 10 μg of human transferrin |
| | injected vs peak area |
| | Standard curve used for the results shown |
| | was linear up to 10 μg. |
| | y = 530388.x + 10526.7 where y = peak |
| | area, and x = amount in μg. |
| (r²): | 0.999953, where Correlation Coefficient = r |

### Example 3

### Chromosomal Over-Expression of PDI

S. cerevisiae Strain A was selected to investigate the secretion of recombinant glycosylated transferrin expression from plasmid pDB2506 and recombinant non-glycosylated transferrin (N413Q, N611Q) from plasmid pDB2536. Strain A has the following characteristics—
additional chromosomally integrated PDI1 gene integrated at the host PDI1 chromosomal location.
the URA3 gene and bacterial DNA sequences containing the ampicillin resistance gene were also integrated into the S. cerevisiae genome at the insertion sites for the above genes.

A control strain had none of the above insertions.

Control strain [cir⁰] and Strain A [cir⁰] were transformed to leucine prototrophy with pDB2506 (recombinant transferring, pDB2536 (recombinant non-glycosylated transferrin (N413Q, N611Q)) or pSAC35 (control). Transformants were selected on BMMD-agar.

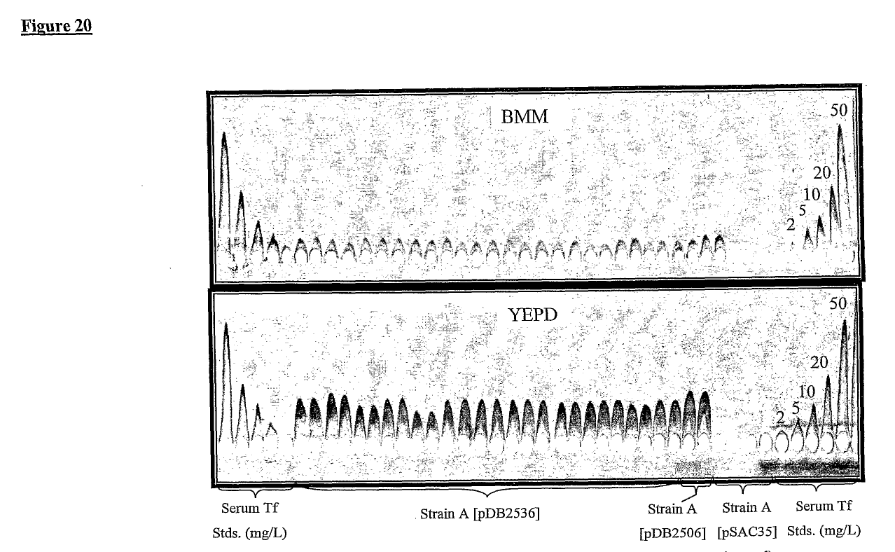

The relative level of transferrin secretion in BMMD shake flask culture was determined for each strain/plasmid combination by rocket immunoelectrophoresis (RIE). FIG. 19 shows that both strains secreted both the glycosylated and non-glycosylated recombinant transferrins into the culture supernatant.

The levels of both the glycosylated and non-glycosylated transferrins secreted from Strain A [pDB2506] and Strain A [pDB2536] respectively, appeared higher than the levels secreted from the control strain. Hence, at least in shake flask culture, PDI1 integrated into the host genome at the PDI1 locus in Strain A has enhanced transferrin secretion.

Furthermore, the increase in transferrin secretion observed between control strain [pDB2536] and Strain A [pDB2536] appeared to be at least a 100% increase by RIE. In contrast, the increase in rHA monomer secretion between control strain [pDB2305] and Strain A [pDB2305] was approximately 20% (data not shown). Therefore, the increase in transferrin secretion due to the additional copy of PDI1 in Strain A was surprising large considering that transferrin has 19 disulphide bonds, compared to rHA with 17 disulphide bonds. Additional copies of the PDI1 gene may be particularly beneficial for the secretion from S. cerevisiae of proteins from the transferrin family, and their derivatives.

The levels of transferrin secreted from Strain A [pDB2536] and Strain A [pDB2506] were compared by RIE for transformants grown in BMMD and YEPD (FIG. 20). Results indicated that a greater than 2-fold increase in titres of both non-glycosylated recombinant transferrin (N413Q, N611Q) and glycosylated recombinant transferrin was achieved by growth in YEPD (10-20 mg.L⁻¹ serum transferrin equivalent) compared to BMMD (2-5 mg.L⁻¹ serum transferrin equivalent). The increase in both glycosylated and non-glycosylated transferrin titre observed in YEPD suggested that both transferrin expression plasmids were sufficiently stable under non-selective growth conditions to allow the expected increased biomass which usually results from growth in YEPD to be translated into increased glycosylated and non-glycosylated transferrin productivity.

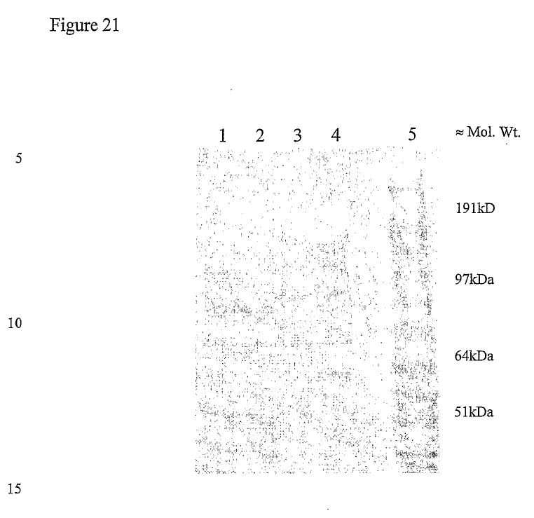

SDS-PAGE analysis of non-glycosylated transferrin (N413Q, N611Q) secreted from Strain A [pDB2536] and glycosylated transferrin from Strain A [pDB2506] grown in BMMD shake flask culture is shown in FIG. 21. Strain A [pDB2536] samples clearly showed an additional protein band compared to the Strain A [pSAC35] control. This extra band migrated at the expected position for the recombinant transferrin (N413 Q, N611 Q) secreted from control strain [pDB2536]. Strain A [pDB2506] culture supernatants appeared to contain a diffuse protein band at the position expected for transferrin. This suggested that the secreted recombinant transferrin was heterogeneous, possibly due to hyper-mannosylation at Asp413 and/or Asp611.

### Example 4

### Comparing Transferrin Secretion from S. Cerevisiae Control Strain Containing pDB2711 with Transferrin Secretion from S. cerevisiae Strain A

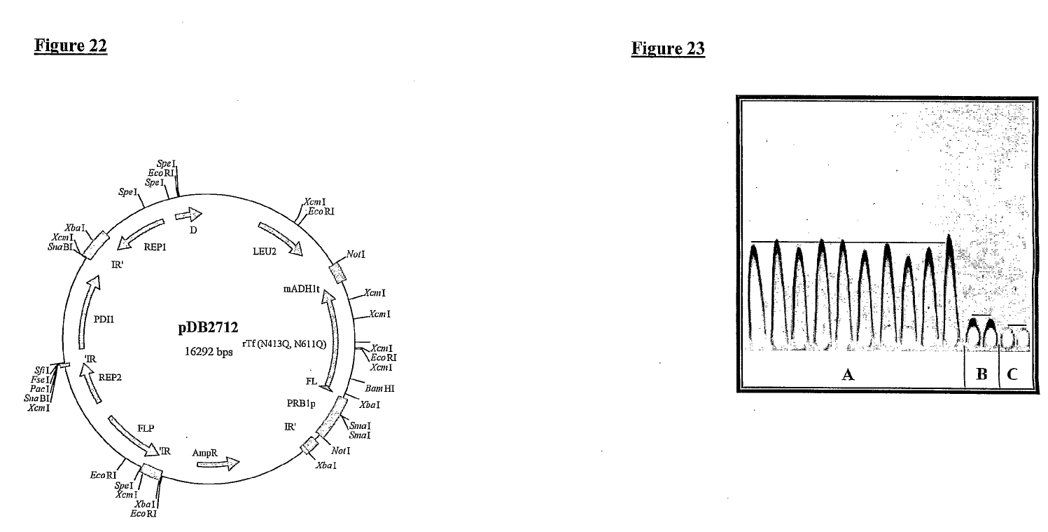

Plasmid pDB2711 is as described above. Plasmid pDB2712 (FIG. 22) was also produced with the NotI cassette in the opposite direction to pDB2711.

Control strain S. cerevisiae [cir⁰] was transformed to leucine prototrophy with pDB2711 and pDB2712. Transformants were selected on BMMD-agar and cryopreserved trehalose stocks of control strain [pDB2711] were prepared.

Secretion of recombinant transferrin (N413Q, N611Q) by control strain [pDB2711], control strain [pDB2712], Strain A [pDB2536], control strain [pDB2536] and an alternative control strain [pDB2536] was compared in both BMMD and YEPD shake flask culture. RIE indicated that a significant increase in recombinant transferrin secretion had been achieved from control strain [pDB2711] with multiple episomal PDI1 copies, compared to Strain A [pDB2536] with two chromosomal copies of PDI1, and control strain [pDB2536] with a single chromosomal copy of PDI1 gene (FIG. 23). Control strain [pDB2711] and control strain [pDB2712] appeared to secrete similar levels of rTf (N413Q, N611Q) into the culture media. The levels of secretion were relatively consistent between control strain [pDB2711] and control strain [pDB2712] transformants in both BMMD and YEPD media, suggesting that plasmid stability was sufficient for high-level transferrin secretion even under non-selective conditions. This is in contrast to the previous published data in relation to recombinant PDGF-BB and HSA where introduction of PDI1 into multicopy 2 μm plasmids was shown to be detrimental to the host.

TABLE 3
Recombinant transferrin titres from high cell density fermentations

Supernatant (g · L⁻¹)

| Strain | GP-HPLC | SDS-PAGE |
|---|---|---|
| Control [pDB2536] | 0.5/0.4 | — |
| Alternative control [pDB2536] | 1.5/1.6 | 0.6 |
| | 0.9/0.9 | 0.4/0.4/0.5 |
| Strain A [pDB2536] | 0.7 | 0.6 |
| | 0.6 | — |
| Control [pDB2711] | 3.5 | 3.6 |
| | 3.4 | 2.7/3.1 |

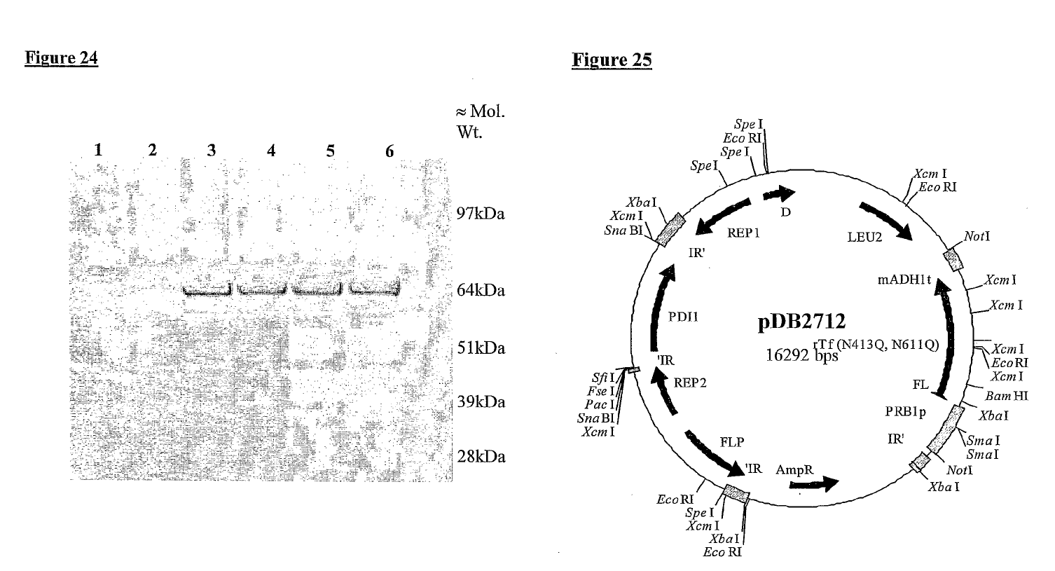

Reducing SDS-PAGE analysis of transferrin secreted from control strain [pDB2711], control strain [pDB2712], Strain A [pDB2536], control strain [pDB2536] and alternative control strain [pDB2536] in BMMD shake flask culture is shown in FIG. 24. This shows an abundant protein band in all samples from control strain [pDB2711] and control strain [pDB2712] at the position expected for transferrin (N413Q, N611Q). The relative stain intensity of the transferrin (N413Q, N611Q) band from the different strains suggested that Strain A [pDB2536] produced more than control strain [pDB2536] and alternative control strain [pDB2536], but that there was an even more dramatic increase in secretion from control strain [pDB2711] and control strain [pDB2712]. The increased recombinant transferrin secretion observed was concomitant with the increased PDI1 copy number in these strains. This suggested that Pdi1p levels were limiting transferrin secretion in control strain, Strain A and the alternative control strain, and that elevated PDI1 copy number was responsible for increased transferrin secretion. Elevated PDI1 copy number could increase the steady state expression level of PDI1 so increasing the amount of Pdi1p activity. There are a number of alternative methods by which this could be achieved without increasing the copy number of the PDI1 gene, for example the steady state PDI1 mRNA level could be increased by either increasing the transcription rate, say by use of a higher efficiency promoter, or by reducing the clearance rate of the PDI1 mRNA. Alternatively, protein engineering could be used to enhance the specific activity or turnover number of the Pdi1p protein.

In high cell density fermentations control strain [pDB2711] recombinant transferrin (N413Q, N611Q) production was measured at approximately 3 g.L⁻¹ by both GP-HPLC analysis and SDS-PAGE analysis (Table 3). This level of production is several fold-higher than control strain, the alternative control strain or Strain A containing pDB2536. Furthermore, for the production of proteins for therapeutic use in humans, expression systems such as control strain [pDB2711] have advantages over those using Strain A, as they do not contain bacterial DNA sequences.

### CONCLUSIONS

Secretion of recombinant transferrin from a multicopy expression plasmid (pDB2536) was investigated in S. cerevisiae strains containing an additional copy of the PDI1 gene integrated into the yeast genome. Transferrin secretion was also investigated in S. cerevisiae transformed with a multicopy expression plasmid, in which the PDI1 gene has been inserted into the multicopy episomal transferrin expression plasmid (pDB2711).

A S. cerevisiae strain with an additional copy of the PDI1 gene integrated into the genome at the endogenous PDI1 locus, secreted recombinant transferrin and non-glycosylated recombinant transferrin (N413Q, N611Q) at an elevated level compared to strains containing a single copy of PDI1. A further increase in PDI1 copy number was achieved by using pDB2711 In high cell density fermentation of the strain transformed with pDB2711, recombinant transferrin (N413Q, N611Q) was secreted at approximately 3 g.L⁻¹, as measured by SDS-PAGE and GP-HPLC analysis. Therefore, increased PDI1 gene copy number has produced a large increase in the quantity of recombinant transferrins secreted from S. cerevisiae.

The following conclusions are drawn—

1. In shake flask analysis of recombinant transferrin expression from pDB2536 (non-glycosylated transferrin (N413Q, N611Q) and pDB2506 (glycosylated transferrin) the S. cerevisiae strain Strain A secreted higher levels of both recombinant transferrins into the culture supernatant than control strains. This was attributed to the extra copy of PDI1 integrated at the PDI1 locus.

2. Control strain [pDB2711], which contained the PDI1 gene on the multicopy expression plasmid, produced a several-fold increase in recombinant transferrin (N413Q, N611Q) secretion compared to Strain A [pDB2536] in both shake flask culture and high cell density fermentation.

3. Elevated PDI1 copy number in yeast such as S. cerevisiae will be advantageous during the production of heterologous proteins, such as those from the transferrin family.

4. pSAC35-based plasmids containing additional copies of PDI1 gene have advantages for the production of proteins from the transferrin family, and their derivatives, such as fusions, mutants, domains and truncated forms.

### Example 5

### Insertion of a PDI1 Gene Into a 2 μm-Like Plasmid Increased Secretion of Recombinant Transferrin from Various Different S. cerevisiae Strains

The S. cerevisiae strain JRY188 cir⁺ (National Collection of Yeast Cultures) and MT302/28B cir⁺ (Finnis et al., 1993, Eur. J. Biochem., 212, 201-210) was cured of the native 2 μm plasmid by galactose induced over-expression of FLP from Yep351-GAL-FLP1, as described by Rose and Broach (1990, Meth. Enzymol., 185, 234-279) to create the S. cerevisiae strains JRY188 cir⁰ and MT302/28B cir⁰, respectively.

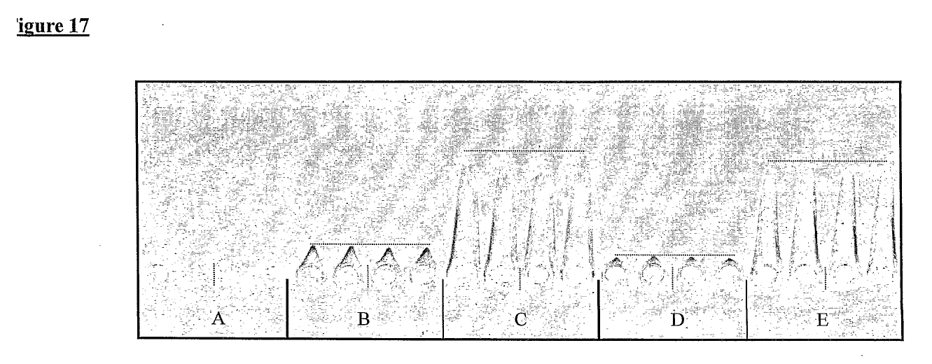

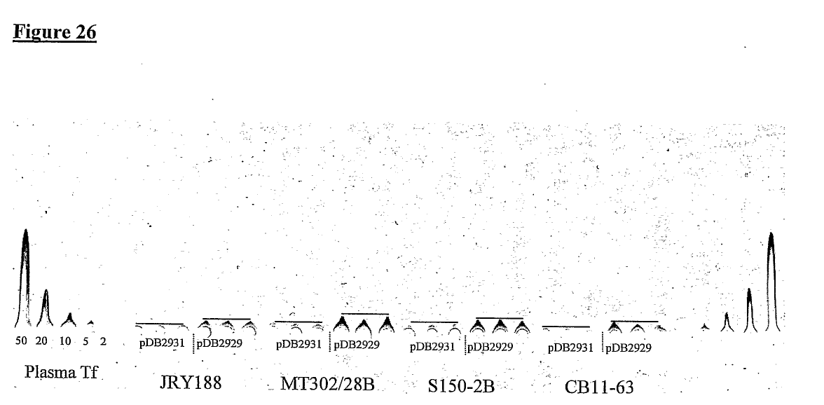

The S. cerevisiae strains JRY188 cir⁰, MT302/28B cir⁰, S150-2B cir⁰ (Cashmore et al., 1986, Mol. Gen. Genet., 203, 154-162), CB11-63 cir⁰ (Zealey et al., 1988, Mol. Gen. Genet., 211, 155-159) were all transformed to leucine prototrophy with pDB2931 (FIG. 14) and pDB2929 (FIG. 12). Transformants were selected on appropriately supplemented minimal media lacking leucine. Transformants of each strain were inoculated into 10 mL YEPD in 50 mL shake flasks and incubated in an orbital shaker at 30° C., 200 rpm for 4-days. Culture supernatants were harvested and the recombinant transferrin titres compared by rocket immunoelectrophoresis (FIG. 26). The results indicated that the transferrin titres in supernatants from all the yeast strains were higher when PDI1 was present in the 2 μm plasmid (pDB2929) than when it was not (pDB2931)

### Example 6

### The Construction of Expression Vectors Containing Various PDI1 Genes and the Expression Cassettes for Various Heterologous Proteins on the Same 2 μm-Like Plasmid

PCR Amplification and Cloning of PDI1 Genes into YIplac211:

The PDI1 genes from S. cerevisiae S288c and S. cerevisiae SKQ2n were amplified by PCR to produce DNA fragments with different lengths of the 5′-untranslated region containing the promoter sequence. PCR primers were designed to permit cloning of the PCR products into the EcoRI and BamHI sites of YIplac211 (Gietz & Sugino, 1988, Gene, 74, 527-534). Additional restriction endonuclease sites were also incorporated into PCR primers to facilitate subsequent cloning. Table 4 describes the plasmids constructed and Table 5 gives the PCR primer sequences used to amplify the PDI1 genes. Differences in the PDI1 promoter length within these YIplac211-based plasmids are described in Table 4.

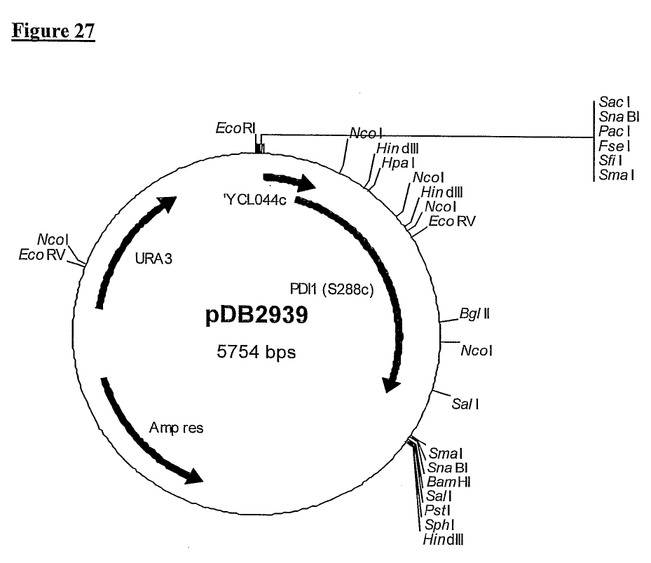

pDB2939 (FIG. 27) was produced by PCR amplification of the PDI1 gene from S. cerevisiae S288c genomic DNA with oligonucleotide primers DS248 and DS250 (Table 5), followed by digesting the PCR product with EcoRI and BamHI and cloning the approximately 1.98-kb fragment into YIplac211 (Gietz & Sugino, 1988, Gene, 74, 527-534), that had been cut with EcoRI and BamHI. DNA sequencing of pDB2939 identified a missing ‘G’ from within the DS248 sequence, which is marked in bold in Table 5. Oligonucleotide primers used for sequencing the PDI1 gene are listed in Table 6, and were designed from the published S288c PDI1 gene sequence (PDI1/YCL043C on chromosome III from coordinates 50221 to 48653 plus 1000 basepairs of upstream sequence and 1000 basepairs of downstream sequence. (http://www.yeastgenome.org/ Genebank Accession number NC001135).

TABLE 4
YIplac211-based Plasmids Containing PDI1 Genes

| | Plasmid | PDI1 Gene | | | |
|---|---|---|---|---|---|
| Plasmid | Base | Source | Promoter | Terminator | PCR Primers |
| pDB2939 | YIplac211 | S288c | Long (˜210-bp) | → Bsu36I | DS248 + DS250 |
| pDB2941 | YIplac211 | S288c | Medium (˜140-bp) | → Bsu36I | DS251 + DS250 |
| pDB2942 | YIplac211 | S288c | Short (˜80-bp) | → Bsu36I | DS252 + DS250 |
| pDB2943 | YIplac211 | SKQ2n | Long (˜210-bp) | → Bsu36I | DS248 + DS250 |
| pDB2963 | YIplac211 | SKQ2n | Medium (˜140-bp) | → Bsu36I | DS267 + DS250 |
| pDB2945 | YIplac211 | SKQ2n | Short (˜80-bp) | → Bsu36I | DS252 + DS250 |

TABLE 5

Oligonucleotide Primers for PCR Amplification
of S. cerevisiae PDII Genes

| | |
|---|---|
| Primer | Sequence |

| | |
|---|---|
| DS248 | 5′-GTCAGAATTCGAGCTCTACGTATTAATTAAGGCCGGCCAGG |
| | CCCGGGCTAGTCTCTTTTTCCAATTTGCCACCGTGTAGCATTTT |
| | GTTGT-3′ |
| DS249 | 5′-GTCAGGATCCTACGTACCCGGGGATATCATTATCATCTTTG |
| | TCGTGGTCATCTTGTGTG-3′ |
| DS250 | 5′-GTCAGGATCCTACGTACCCGGGTAAGGCGTTCGTGCAGTGT |
| | GACGAATATAGCG-3′ |
| DS251 | 5′-GTCAGAATTCGAGCTCTACGTATTAATTAAGGCCGGCCAGG |
| | CCCGGGCCCGTATGGACATACATATATATATATATATATATATA |
| | TATTTTGTTACGCG-3′ |
| DS252 | 5′-GTCAGAATTCGAGCTCTACGTATTAATTAAGGCCGGCCAGG |
| | CCCGGGCTTGTTGCAAGCAGCATGTCTAATTGGTAATTTTAAAG |
| | CTGCC-3′ |
| DS267 | 5′-GTCAGAATTCGAGCTCTACGTATTAATTAAGGCCGGCCAGG |
| | CCCGGGCCCGTATGGACATACATATATATATATATATATATATA |
| | TATATATTTTGTTACGCG-3′ |

TABLE 6

Oligonucleotide Primers for DNA Sequencing
S. cerevisiae PDII Genes

| | |
|---|---|
| Primer | Sequence |

| | |
|---|---|
| DS253 | 5′-CCTCCCTGCTGCTCGCC-3′ |
| DS254 | 5′-CTGTAAGAACATGGCTCC-3′ |
| DS255 | 5′-CTCGATCGATTACGAGGG-3′ |
| DS256 | 5′-AAGAAAGCCGATATCGC-3′ |
| DS257 | 5′-CAACTCTCTGAAGAGGCG-3′ |
| DS258 | 5′-CAACGCCACATCCGACG-3′ |
| DS259 | 5′-GTAATTCTGATCACTTTGG-3′ |
| DS260 | 5′-GCACTTATTATTACTACGTGG-3′ |
| DS261 | 5′-GTTTTCCTTGATGAAGTCG-3′ |
| DS262 | 5′-GTGACCACACCATGGGGC-3′ |
| DS263 | 5′-GTTGCCGGCGTGTCTGCC-3′ |
| DS264 | 5′-TTGAAATCATCGTCTGCG-3′ |
| DS265 | 5′-CGGCAGTTCTAGGTCCC-3′ |
| DS266 | 5′-CCACAGCCTCTTGTTGGG-3′ |
| M13/pUC Primer (−40) | 5′-GTTTTCCCAGTCACGAC-3′ |

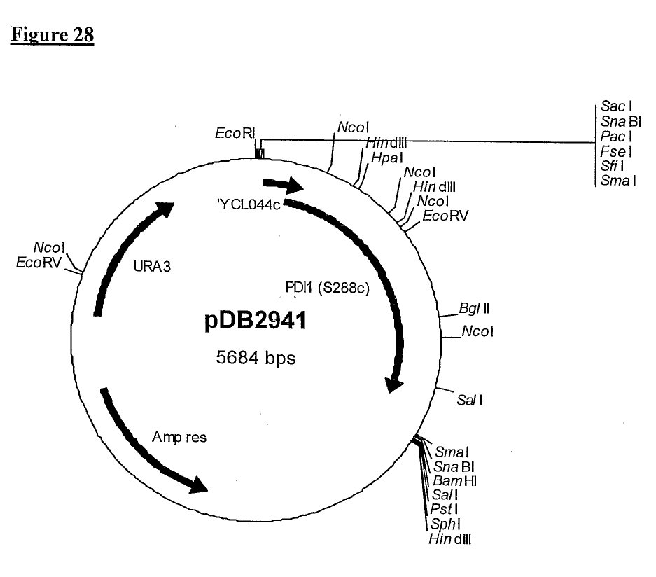

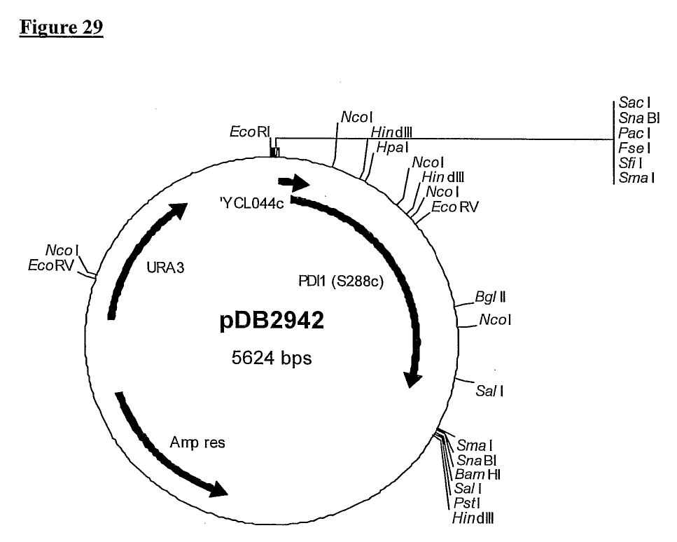

Plasmids pDB2941 (FIG. 28) and pDB2942 (FIG. 29) were constructed similarly using the PCR primers described in Tables 4 and 5, and by cloning the approximately 1.90-kb and 1.85-kb EcoRI-BamHI fragments, respectively, into YIplac211. The correct DNA sequences were confirmed for the PDI1 genes in pDB2941 and pDB2942.

The S. cerevisiae SKQ2n PDI1 gene sequence was PCR amplified from plasmid DNA containing the PDI1 gene from pMA3a:C7 (U.S. Pat. No. 6,291,205), also known as Clone C7 (Crouzet & Tuite, 1987, supra; Farquhar et al, 1991, supra). The SKQ2n PDI1 gene was amplified using oligonucleotide primers DS248 and DS250 (Tables 4 and 5). The approximately 2.01-kb PCR product was digested with EcoRI and BamHI and ligated into YIplac211 (Gietz & Sugino, 1988, Gene, 74, 527-534) that has been cut with EcoRI and BamHI, to produce plasmid pDB2943 (FIG. 30). The 5′ end of the SKQ2n PDI1 sequence is analogous to a blunt-ended SpeI-site extended to include the EcoRI, SacI, SnaBI, PacI, FseI, SfiI and SmaI sites, the 3′ end extends up to a site analogous to a blunt-ended Bsu36I site, extended to include a SmaI, SnaBI and BamHI sites. The PDI1 promoter length is approximately 210 bp. The entire DNA sequence was determined for the PDI1 fragment using oligonucleotide primers given in Table 6. This confirmed the presence of a coding sequence for the PDI protein of S. cerevisiae strain SKQ2n (NCBI accession number CAA38402), but with a serine residue at position 114 (not an arginine residue as previously published). Similarly, in the same way as in the S. cerevisiae S288c sequence in pDB2939, pDB2943 also had a missing ‘G’ from within the DS248 sequence, which is marked in bold in Table 5.

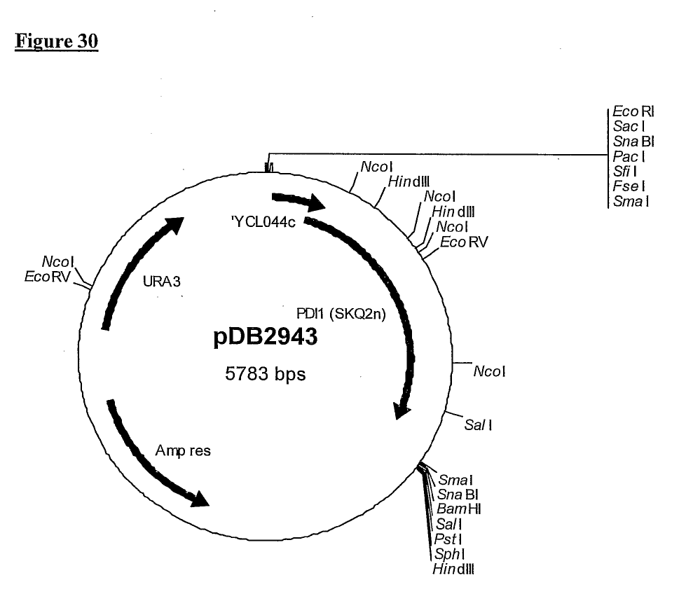

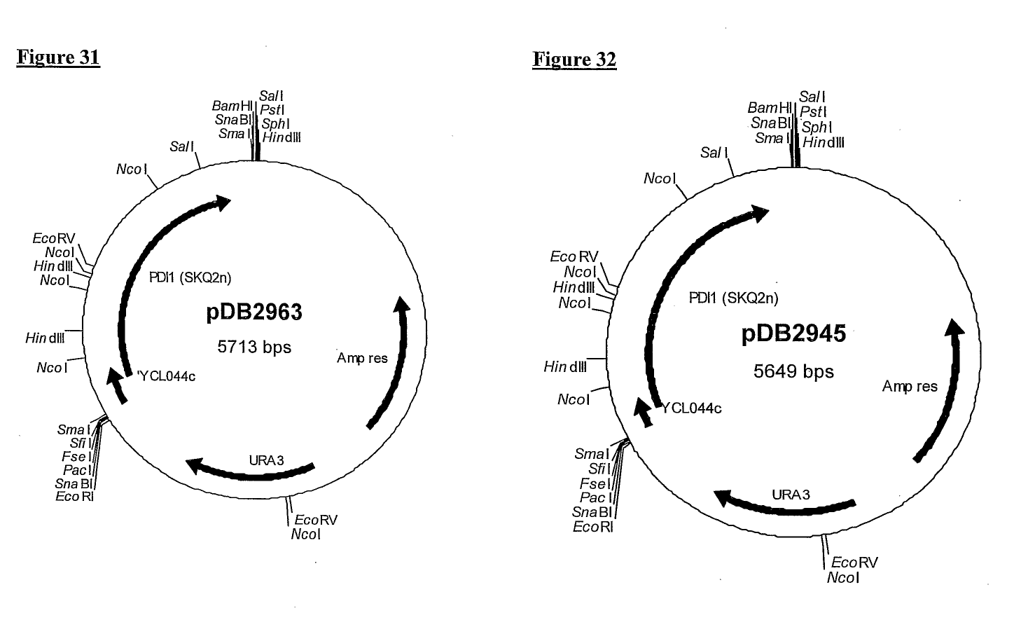

Plasmids pDB2963 (FIG. 31) and pDB2945 (FIG. 32) were constructed similarly using the PCR primers described in Tables 4 and 5, and by cloning the approximately 1.94-kb and 1.87-kb EcoRI-BamHI fragments, respectively, into YIplac211. The expected DNA sequences were confirmed for the PDI1 genes in pDB2963 and pDB2945, with a serine codon at the position of amino acid 114.

The Construction of pSAC35-Based rHA Expression Plasmids with Different PDI1 Genes Inserted at the XcmI-Site After REP2:

pSAC35-based plasmids were constructed for the co-expression of rHA with different PDI1 genes (Table 7).

TABLE 7
pSAC35-based plasmids for co-expression of rHA with different PDI1 genes

| | | | | | | Heterologous Protein |
|---|---|---|---|---|---|---|
| | Plasmid | PDI1 Gene at XcmI-site after REP2 | | | | Expression Cassette |
| Plasmid | Base | Source | Promoter | Terminator | Orientation | (at NotI-site) |
| pDB2982 | pSAC35 | SKQ2n | Long | → Bsu36I | A | rHA |
| pDB2983 | pSAC35 | SKQ2n | Long | → Bsu36I | B | rHA |
| pDB2984 | pSAC35 | SKQ2n | Medium | → Bsu36I | A | rHA |
| pDB2985 | pSAC35 | SKO2n | Medium | → Bsu36I | B | rHA |
| pDB2986 | pSAC35 | SKQ2n | Short | → Bsu36I | A | rHA |
| pDB2987 | pSAC35 | SKQ2n | Short | → Bsu36I | B | rHA |
| pDB2976 | pSAC35 | S288c | Long | → Bsu36I | A | rHA |
| pDB2977 | pSAC35 | S288c | Long | → Bsu36I | B | rHA |
| pDB2978 | pSAC35 | S288c | Medium | → Bsu36I | A | rHA |
| pDB2979 | pSAC35 | S288c | Medium | → Bsu36I | B | rHA |
| pDB2980 | pSAC35 | S288c | Short | → Bsu36I | A | rHA |
| pDB2981 | pSAC35 | S288c | Short | → Bsu36I | B | rHA |

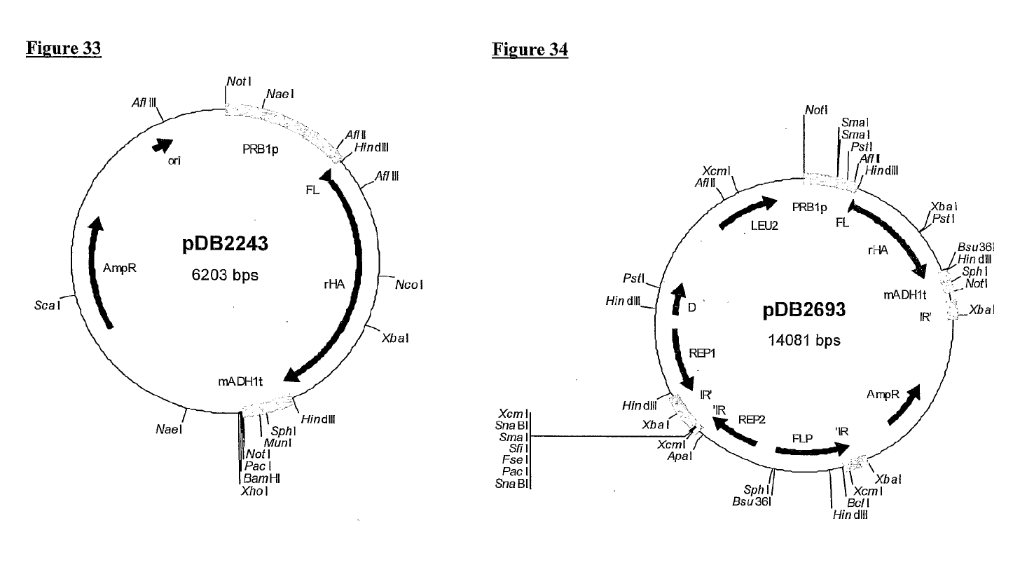

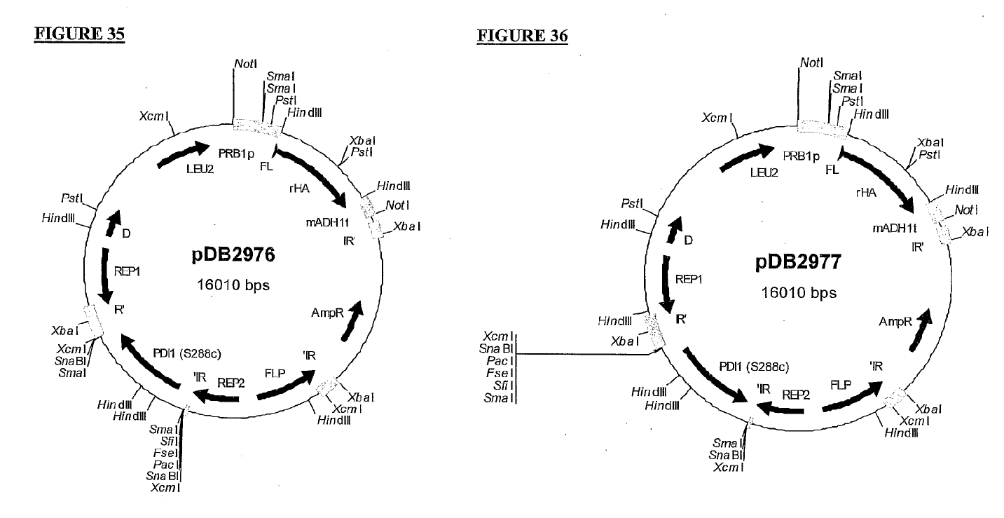

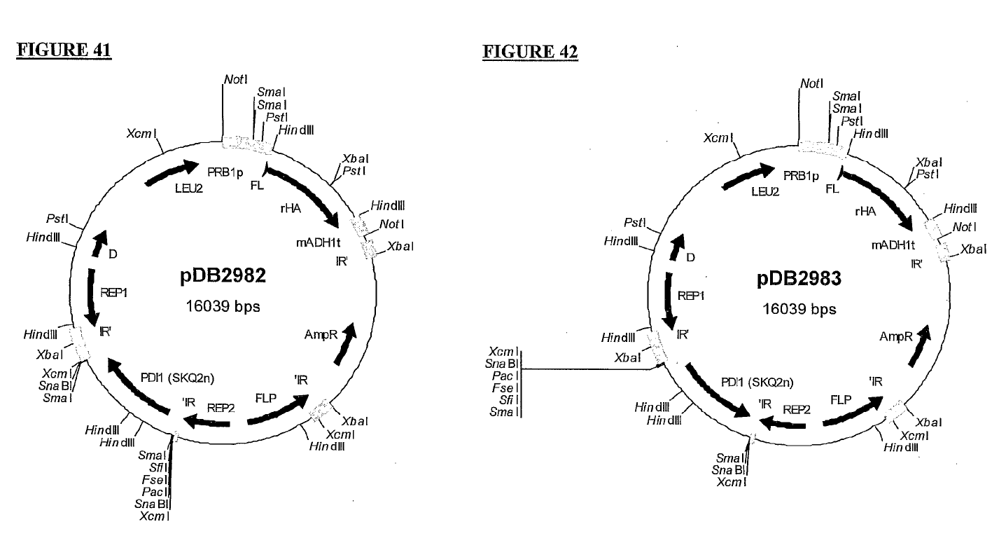

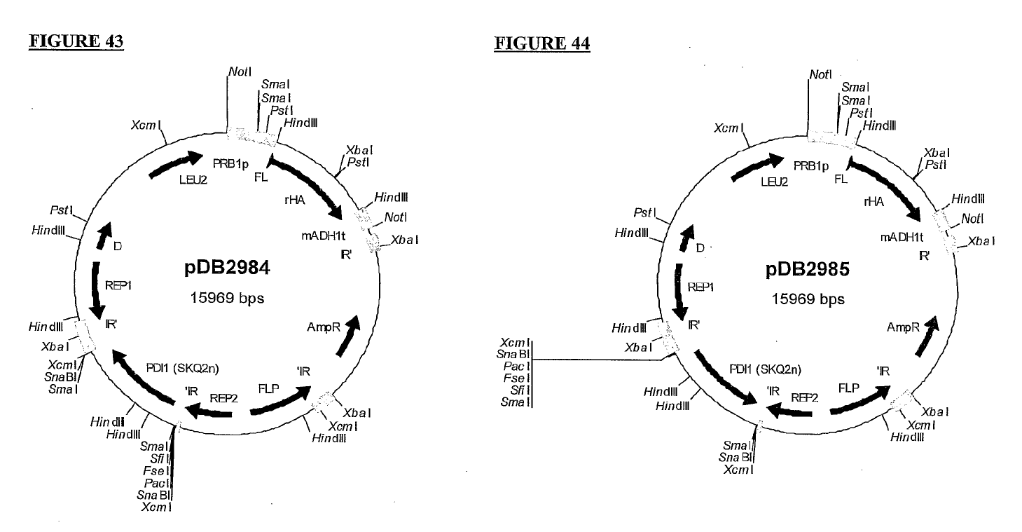

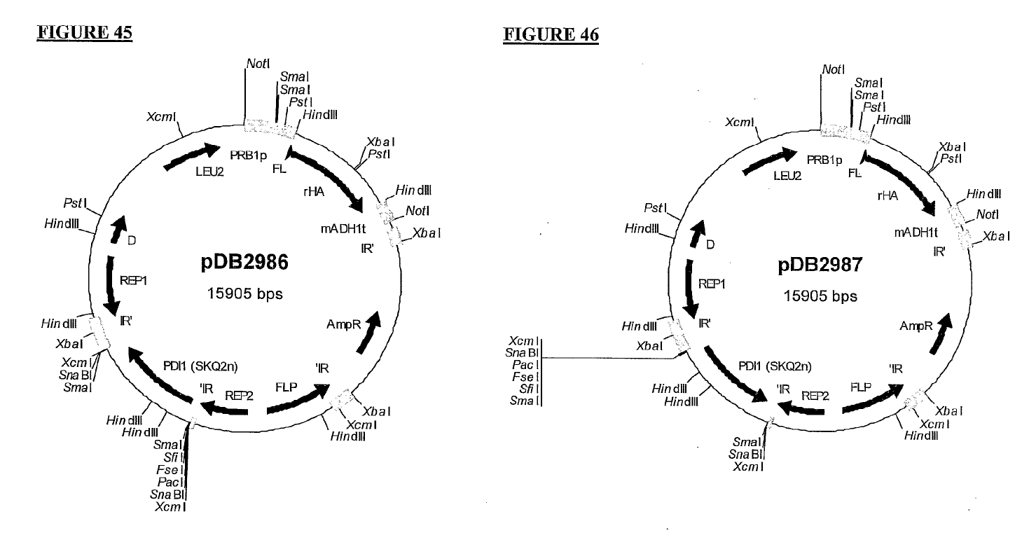

The rHA expression cassette from pDB2243 (FIG. 33, as described in WO 00/44772) was first isolated on a 2,992-bp NotI fragment, which subsequently was cloned into the NotI-site of pDB2688 (FIG. 4) to produce pDB2693 (FIG. 34). pDB2693 was digested with SnaBI, treated with calf intestinal alkaline phosphatase, and ligated with SnaBI fragments containing the PDI1 genes from pDB2943, pDB2963, pDB2945, pDB2939, pDB2941 and pDB2942. This produced plasmids pDB2976 to pDB2987 (FIGS. 35 to 46). PDI1 transcribed in the same orientation as REP2 was designated “orientation A”, whereas PDI1 transcribed in opposite orientation to REP2 was designated “orientation B” (Table 7).

The Construction of pSAC35-Based Transferrin Expression Plasmids with Different PDI1 Genes Inserted at the XcmI-Site After REP2:

pSAC35-based plasmids were constructed for the co-expression of recombinant transferrin (N413Q, N611Q) with different PDI1 genes (Table 8).

TABLE 8
pSAC35-based plasmids for co-expression of transferrin with different PDI1 genes

| | | | | | | Heterologous Protein |
|---|---|---|---|---|---|---|
| | Plasmid | PDI1 Gene at XcmI-site after REP2 | | | | Expression Cassette |
| Plasmid | Base | Source | Promoter | Terminator | Orientation | (at NotI-site) |
| pDB2929 | pSAC35 | SKQ2n | Long | → Bsu36I | A | rTf (N413Q, N611Q) |
| pDB3085 | pSAC35 | S288c | Long | → Bsu36I | A | rTf (N413Q, N611Q) |
| pDB3086 | pSAC35 | S288c | Medium | → Bsu36I | A | rTf (N413Q, N611Q) |
| pDB3087 | pSAC35 | S288c | Short | → Bsu36I | A | rTf (N413Q, N611Q) |

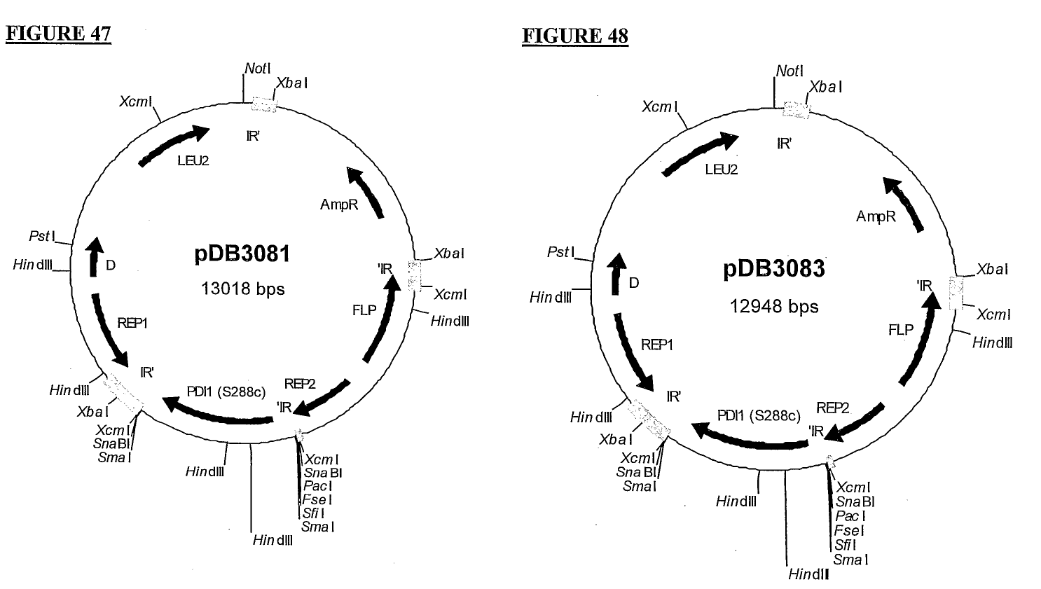

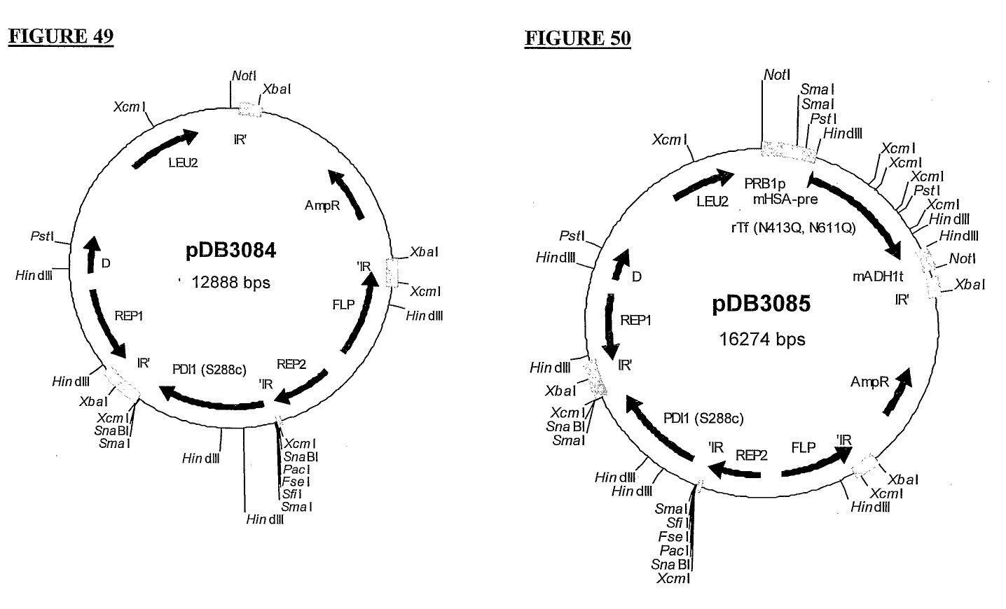

In order to achieve this, the NotI expression cassettes for rHA expression were first deleted from pDB2976, pDB2978, and pDB2980 by NotI digestion and circularisation of the vector backbone. This produced plasmids pDB3081 (FIG. 47), pDB3083 (FIG. 48) and pDB3084 (FIG. 49) as described in Table 9.

TABLE 9
pSAC35-based plasmids with different PDI1 genes

| | | | | | | Heterologous Protein |
|---|---|---|---|---|---|---|
| | Plasmid | PDI1 Gene at XcmI-site after REP2 | | | | Expression Cassette |
| Plasmid | Base | Source | Promoter | Terminator | Orientation | (at NotI-site) |
| pDB2690 | pSAC35 | SKQ2n | Long | → Bsu36I | A | None |
| pDB3081 | pSAC35 | S288c | Long | → Bsu36I | A | None |
| pDB3083 | pSAC35 | S288c | Medium | → Bsu36I | A | None |
| pDB3084 | pSAC35 | S288c | Short | → Bsu36I | A | None |

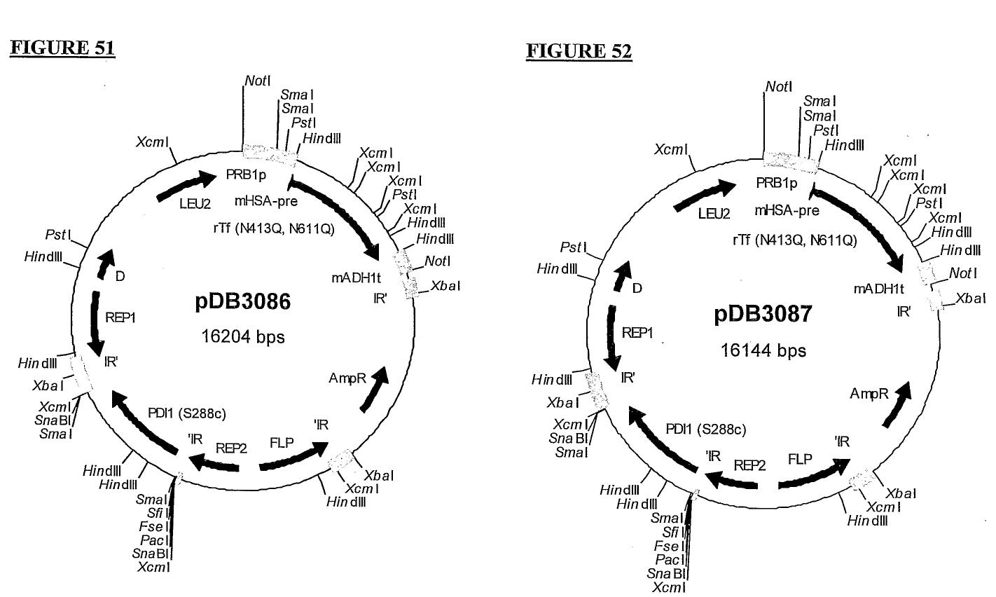

The 3,256-bp NotI fragment from pDB2928 (FIG. 11) was cloned into the NotI-sites of pDB3081, pDB3083 and pDB3084, such that transcription from the transferrin gene was in the same direction as LEU2. This produced plasmids pDB3085 (FIG. 50), pDB3086 (FIG. 51) and pDB3087 (FIG. 52) as described in Table 8.

### Example 7

### Insertion and Optimisation of a PDI1 Gene in the 2 μm-Like Plasmid Increased the Secretion of Recombinant Human Serum Albumin by Various Different S. cerevisiae Strains

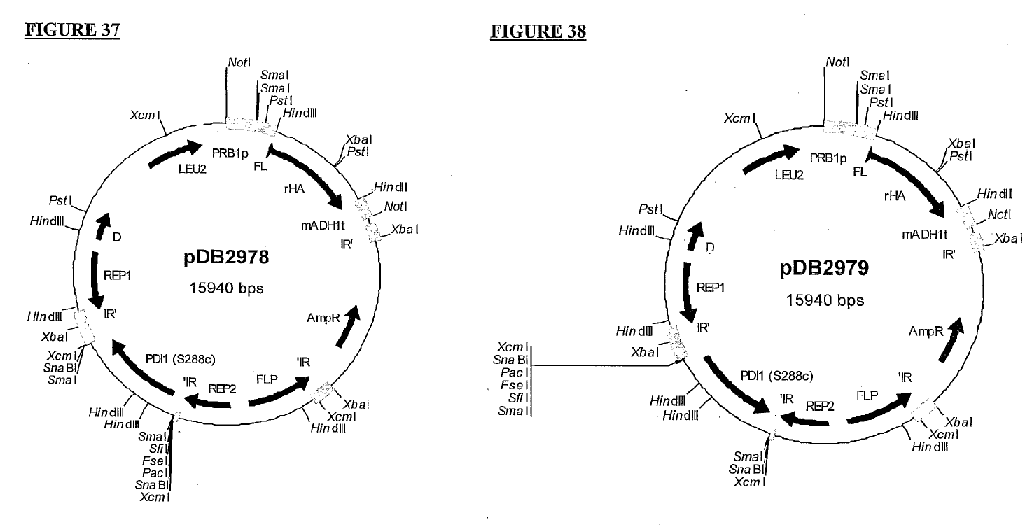

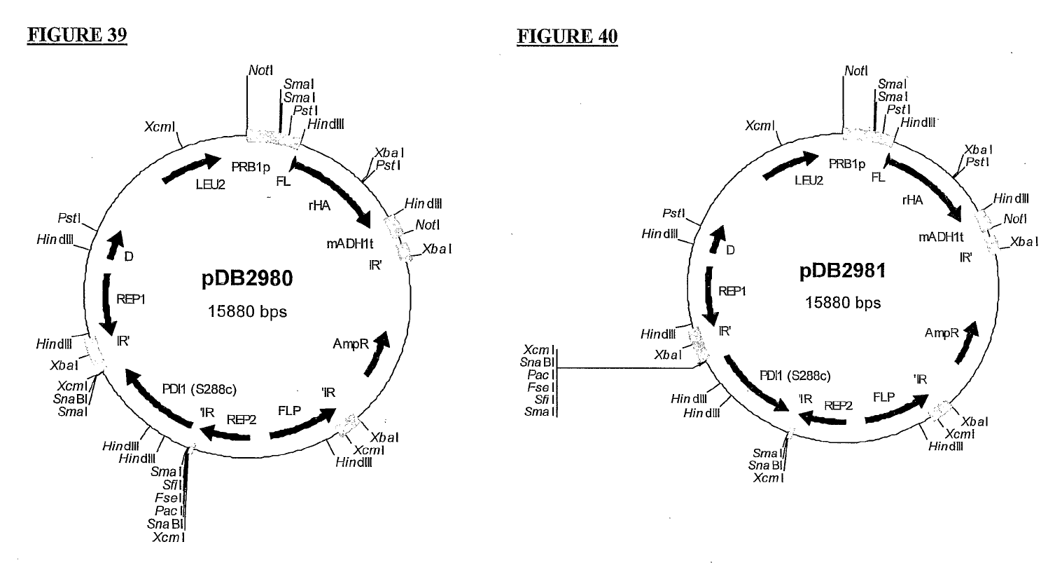

The S. cerevisiae strains JRY188 cir⁰, MT302/28B cir⁰, S150-2B cir⁰, CB11-63 cir⁰ (all described above), AH22 cir⁰ (Mead et al., 1986, Mol. Gen. Genet., 205, 417-421) and DS569 cir⁰ (Sleep et al., 1991, Bio/Technology, 9, 183-187) were transformed to leucine prototrophy with either pDB2244 (WO 00/44772), pDB2976 (FIG. 35), pDB2978 (FIG. 37) or pDB2980 (FIG. 39) using a modified lithium acetate method (Sigma yeast transformation kit, YEAST-1, protocol 2; (Ito et al, 1983, J. Bacteriol., 153, 163; Elble, 1992, Biotechniques, 13, 18)). Transformants were selected on BMMD-agar plates with appropriate supplements, and were subsequently patched out on BMMD-agar plates with appropriate supplements.

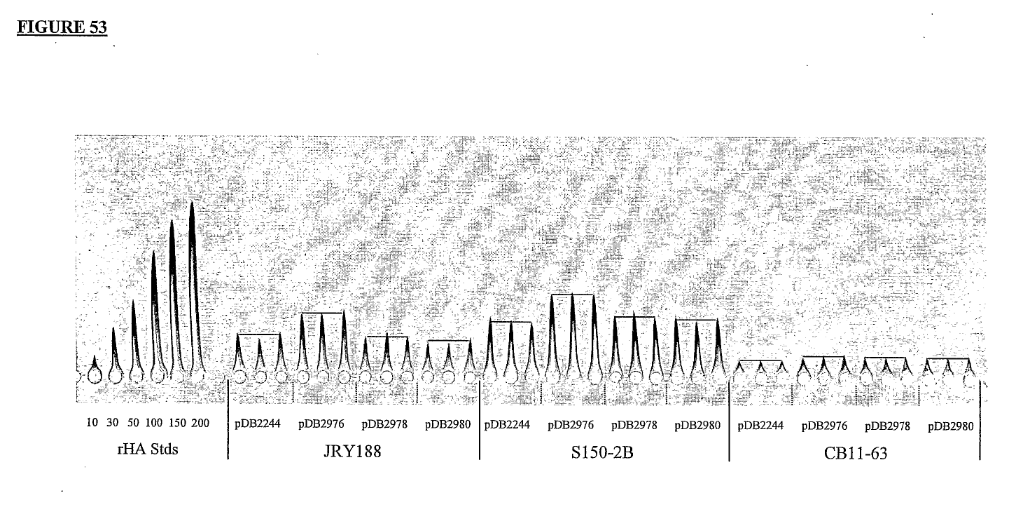

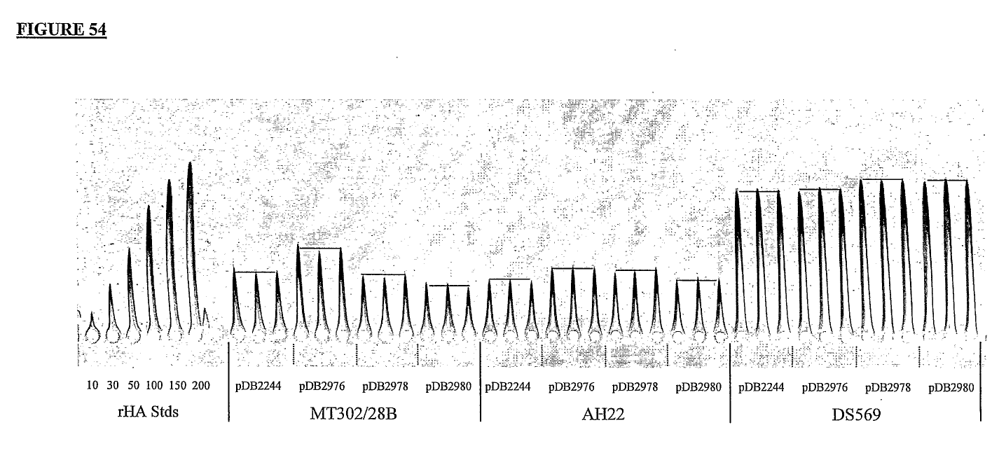

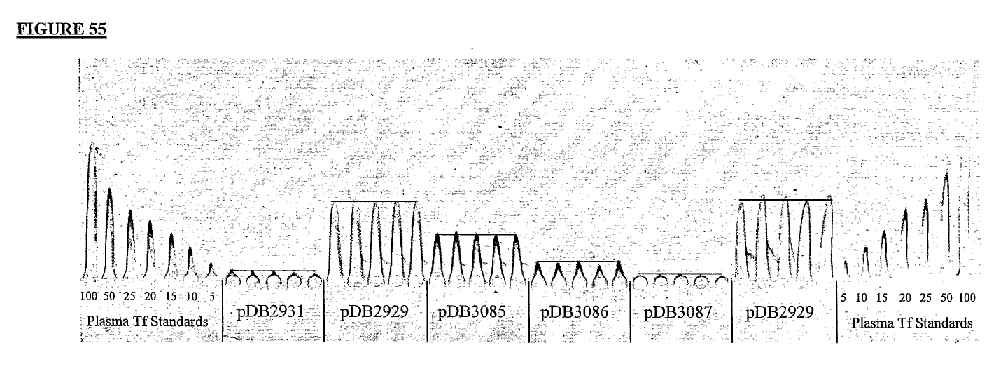

Transformants of each strain were inoculated into 10 mL YEPD in 50 mL shake flasks and incubated in an orbital shaker at 30° C., 200 rpm for 4-days. Culture supernatants were harvested and the recombinant albumin titres compared by rocket immunoelectrophoresis (FIGS. 53 and 54). The results indicated that the albumin titres in the culture supernatants from all the yeast strains were higher when PDI1 was present in the 2 μm plasmid than when it was not (pDB2244). The albumin titre in the culture supernatants in the absence of PDI1 on the plasmid was dependant upon which yeast strain was selected as the expression host, however, in most examples tested the largest increase in expression was observed when PDI1 with the long promoter (˜210-bp) was present in the 2 μm plasmid (pDB2976). Modifying the PDI1 promoter by shortening, for example to delete regulation sites, had the affect of controlling the improvement. For one yeast strain, known to be a high rHA producing strain (DS569) a shorter promoter was preferred for optimal expression.

### Example 8

### Different PDI1 Genes Enhanced the Secretion of Recombinant Transferrin when Co-Expressed on a 2 μm-Based Plasmid

The secretion of recombinant transferrin (N413 Q, N611Q) was investigated with co-expression of the S. cerevisiae SKQ2n PDI1 gene with the long promoter (˜210-bp), and the S. cerevisiae S288c PDI1 with the long, medium and short promoters (˜210 bp, ˜140 bp and ˜80 bp respectively).

The same Control Strain as used in previous examples (e.g. Example 2) was transformed to leucine prototrophy with pDB2931 (negative control plasmid without PDI1) and pDB2929, pDB3085, pDB3086 and pDB3087 (Table 8). Transformants were selected on BMMD-agar plates and five colonies selected for analysis. Strains were grown in 10 mL BMMD and 10 mL YEPD shake flask cultures for 4-days at 30° C., 200 rpm and culture supernatants harvested for analysis by rocket immunoelectrophoresis (RIE).

FIG. 55 shows that in minimal media (BMMD) the S. cerevisiae SKQ2n PDI1 gene with the long promoter gave the highest rTF (N413Q, N611Q) titres. The S. cerevisiae S288c PDI1 gene gave lower rTF (N413Q, N611Q) titres, which decreased further as the PDI1 promoter length was shortened.

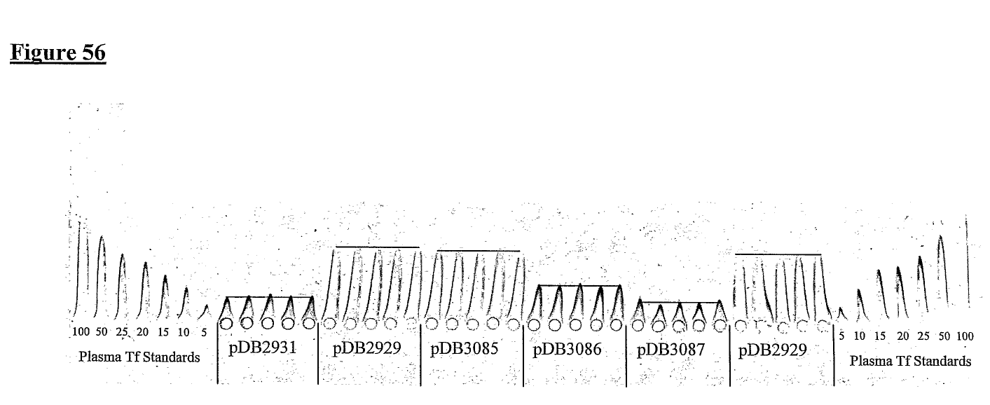

FIG. 56 shows that in rich media (YEPD) the S. cerevisiae SKQ2n PDI1 and S. cerevisiae S288c PDI1 genes with the long promoters gave similar rTF (N413Q, N611Q) production levels. Also, the shorter the promoter length of the S. cerevisiae S288c PDI1 gene the lower was the rTF (N413Q, N611Q) production level.

### Example 9

### PDI1 on the 2 μm-Based Plasmid Enhanced the Secretion of Recombinant Albumin Fusions

The affect of co-expression of the S. cerevisiae SKQ2n PDI1 gene with the long promoter (˜210-bp) upon the expression of recombinant albumin fusions was investigated.

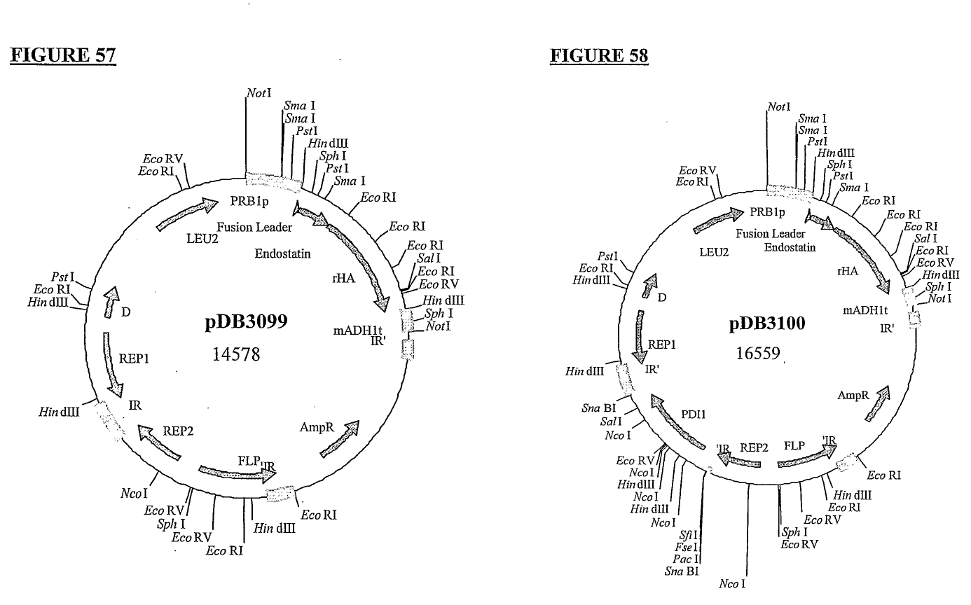

The construction of a NotI N-terminal endostatin-albumin expression cassette (pDB2556) has been previously described (WO 03/066085). Appropriate yeast vector sequences were provide by a “disintegration” plasmid pSAC35 generally disclosed in EP-A-286 424 and described by Sleep, D., et al., 1991, Bio/Technology, 9, 183-187. The 3.54 kb NotI N-terminal endostatin-albumin expression cassette was isolated from pDB2556, purified and ligated into NotI digested pSAC35, which had been treated with calf intestinal phosphatase, creating plasmid pDB3099 containing the NotI expression cassette in the same orientation to the LEU2 selection marker (FIG. 57). An appropriate yeast PDI1 vector sequences were provide by a “disintegration” plasmid pDB2690 (FIG. 6). The 3.54 kb NotI N-terminal endostatin-albumin expression cassette was isolated from pDB2556, purified and ligated into NotI digested pDB2690, which had been treated with calf intestinal phosphatase, creating plasmid pDB3100 containing the NotI expression cassette in the same orientation to the LEU2 selection marker (FIG. 58).

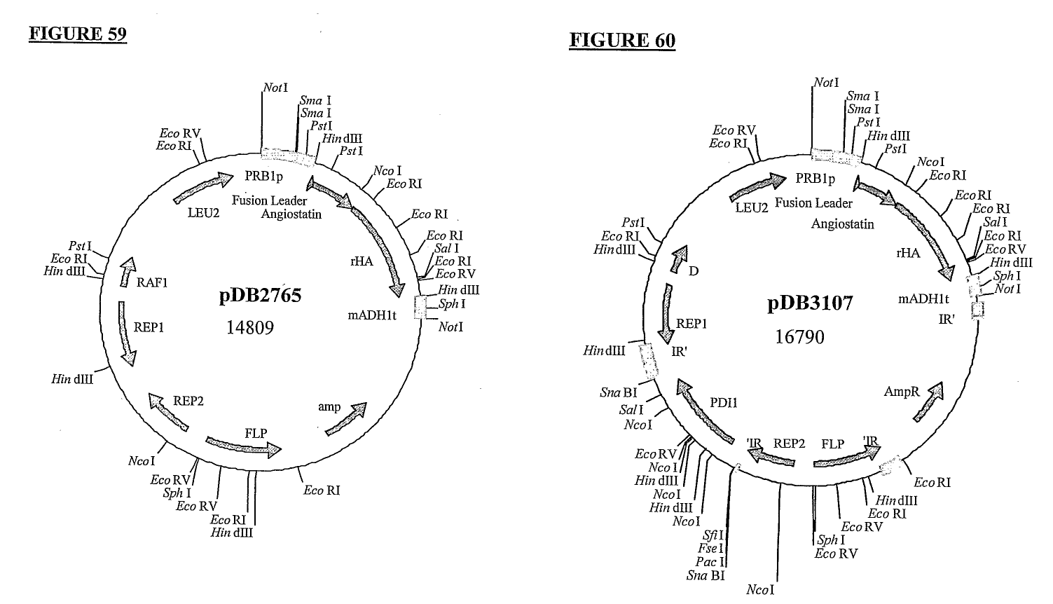

The construction of an NotI N-terminal angiostatin-albumin expression cassette (pDB2556) has been previously described (WO 03/066085), as has the construction of a pSAC35-based yeast expression vector, pDB2765 (FIG. 59). The 3.77 kb NotI N-terminal angiostatin-albumin expression cassette was isolated from pDB2556, purified and ligated into NotI digested pDB2690, an appropriate yeast PDI1 expression vector, which had been treated with calf intestinal phosphatase, creating plasmid pDB3107 containing the NotI expression cassette in the same orientation to the LEU2 selection marker (FIG. 60).

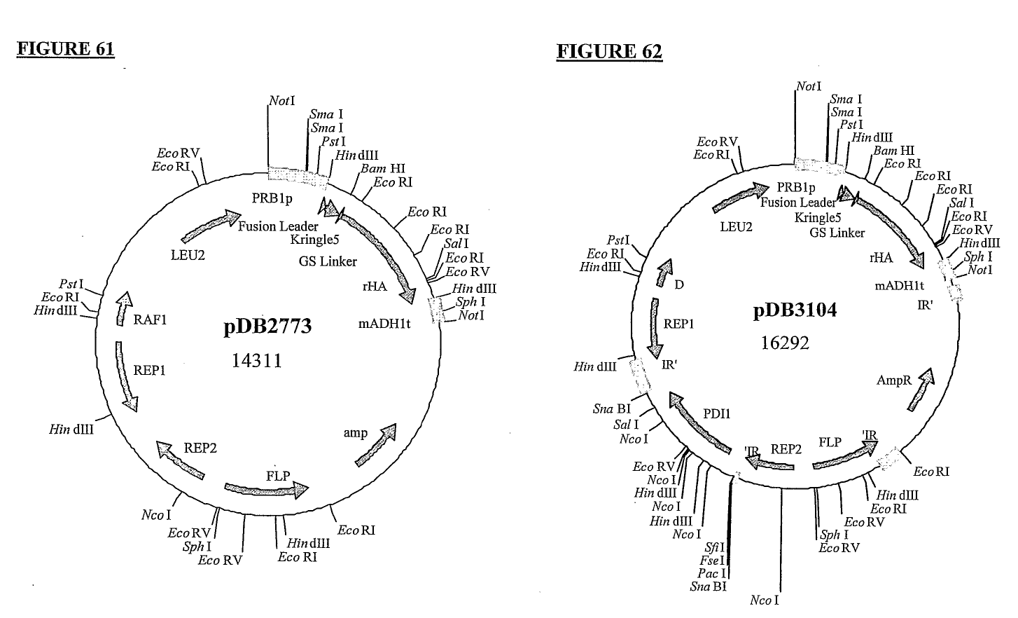

The construction of an NotI N-terminal Kringle5-(GGS)₄GG-albumin expression cassette (pDB2771) has been previously described (WO 03/066085), as has the construction of a pSAC35-based yeast expression vector, pDB2773 (FIG. 61). The 3.27 kb NotI N-terminal Kringle5-(GGS)₄GG-albumin expression cassette was isolated from pDB2771, purified and ligated into NotI digested pDB2690, an appropriate yeast PDI1 expression vector, which had been treated with calf intestinal phosphatase, creating plasmid pDB3104 containing the NotI expression cassette in the same orientation to the LEU2 selection marker (FIG. 62).

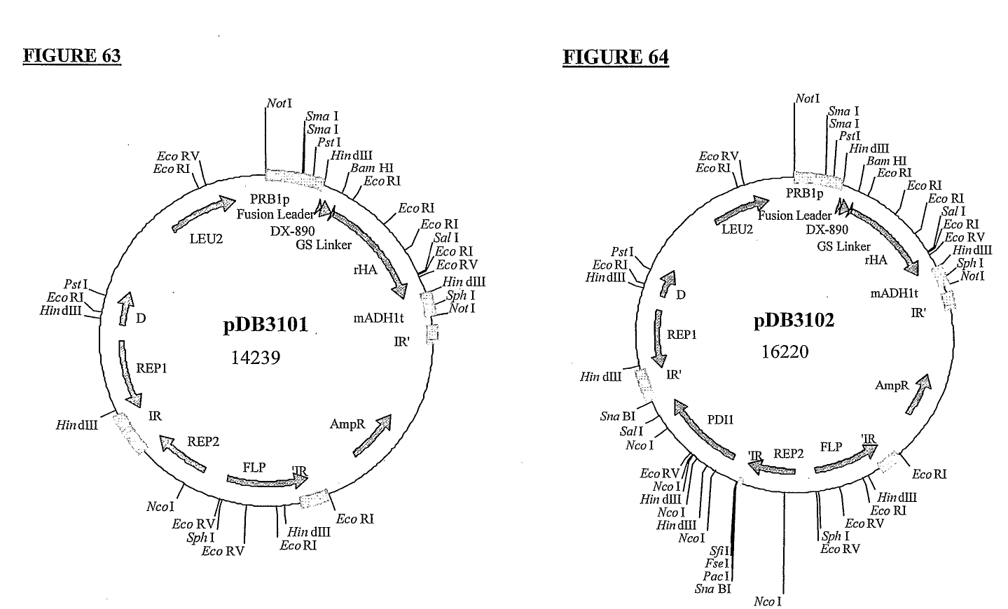

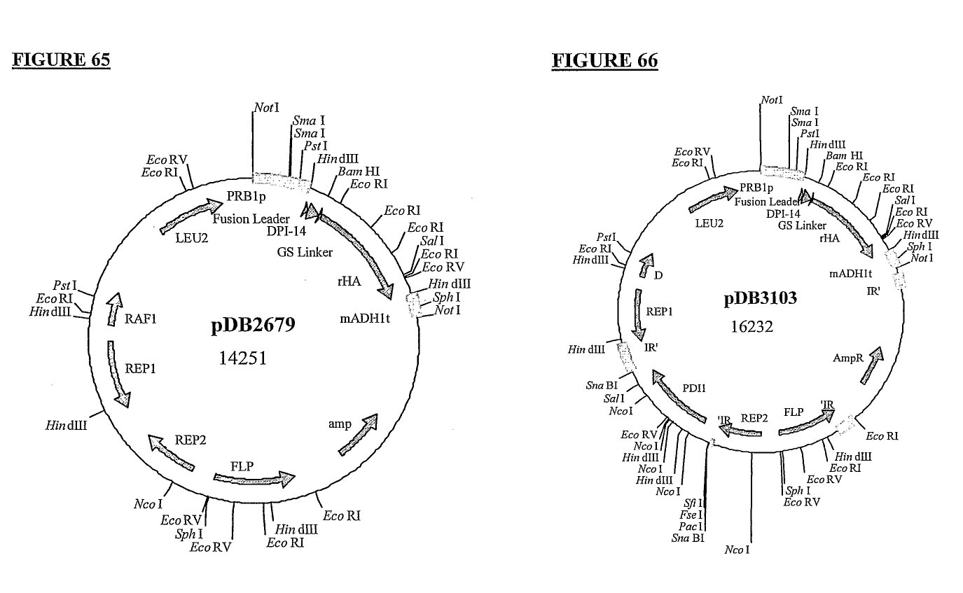

The construction of an NotI N-terminal DX-890-(GGS)₄GG-albumin expression cassette (pDB2683) has been previously described (WO 03/066824). Appropriate yeast vector sequences were provide by the “disintegration” plasmid pSAC35. The 3.20 kb NotI N-terminal DX-890-(GGS)₄GG-albumin expression cassette was isolated from pDB2683, purified and ligated into NotI digested pSAC35, which had been treated with calf intestinal phosphatase, creating plasmid pDB3101 containing the NotI expression cassette in the same orientation to the LEU2 selection marker (FIG. 63). An appropriate yeast PDI1 vector sequences were provide by a “disintegration” plasmid pDB2690 (FIG. 6). The 3.20 kb NotI N-terminal DX-890-(GGS)₄GG-albumin expression cassette was isolated from pDB2683, purified and ligated into NotI digested pDB2690, which had been treated with calf intestinal phosphatase, creating plasmid pDB3102 containing the NotI expression cassette in the same orientation to the LEU2 selection marker (FIG. 64).

The construction of an NotI N-terminal DPI-14-(GGS)₄GG-albumin expression cassette (pDB2666) has been previously described (WO 03/066824), as has the construction of a pSAC35-based yeast expression vector, pDB2679 (FIG. 65).

The 3.21 kb NotI N-terminal DPI-14-(GGS)₄GG-albumin expression cassette was isolated from pDB2666, purified and ligated into NotI digested pDB2690, an appropriate yeast PDI1 expression vector, which had been treated with calf intestinal phosphatase, creating plasmid pDB3103 containing the NotI expression cassette in the same orientation to the LEU2 selection marker (FIG. 66).

CNTF was cloned from human genomic DNA by amplification of the two exons using the following primers for exon 1 and exon 2, respectively, using standard conditions.

Exon 1 primers:
5′-CTCGGTACCCAGCTGACTTGTTTCCTGG-3′;
and
5′-ATAGGATTCCGTAAGAGCAGTCAG-3′
Exon 2 primers:
5′-GTGAAGCATCAGGGCCTGAAC-3′;
and
5′-CTCTCTAGAAGCAAGGAAGAGAGAAGGGAC-3′

Both fragments were ligated under standard conditions, before being re-amplified by PCR using primers 5′-CTCGGTACCCAGCTGACTTGTTTCCTGG-3′ and 5′-CTCTCTAGAAGCAAGGAAGAGAGAAGGGAC-3′ and cloned into vector pCR4 (Invitrogen). To generate Axokine™ (as disclosed in Lambert et al, 2001, PNAS, 98, 4652-4657) site-directed mutagenesis was employed to introduce C17A (TGT→GCT) and Q63R (CAG→AGA) mutations. DNA sequencing also revealed the presence of a silent T→C substitution V85V (GTT→GTC) as described in WO 2004/015113.

The Axokine™ cDNA was amplified by PCR using single stranded oligonucleotides MH33 and MH36 to create an approximate 0.58 kbp PCR fragment.

MH33
5′-ATGCAGATCTTTGGATAAGAGAGCTTTCACAGAGCATTCACCGCTGA
CCCC-3′
MH36
5′-CACCGGATCCACCCCCAGTCTGATGAGAAGAAATGAAACGAAGGTCA
TGG-3′

This was achieved with FastStart Taq DNA polymerase (Roche) in a 50 mL reaction, which was initiated by a 4-minute incubation at 95° C. and followed by 25 cycles of PCR (95° C. for 30 secs, 55° C. for 30 secs, 72° C. for 60 sec). A PCR product of the expected size was observed in a 10 mL sample following electrophoresis in an ethidium bromide stained 1% agarose gel. The remaining PCR product was purified using a QIAquick PCR purification kit (Qiagen) and digested to completion with BamHI and BglII. DNA of approximately the expected size was excised from an ethidium bromide stained 1% (w/v) agarose gel and purified.

Plasmid pDB2573X provided a suitable transcription promoter and terminator, along with a suitable secretory leader sequence and DNA sequences encoding part of a (GGS)₄GG peptide linker fused to the N-terminus of human albumin. The construction of pDB2573X has been previously described (WO 03/066824).

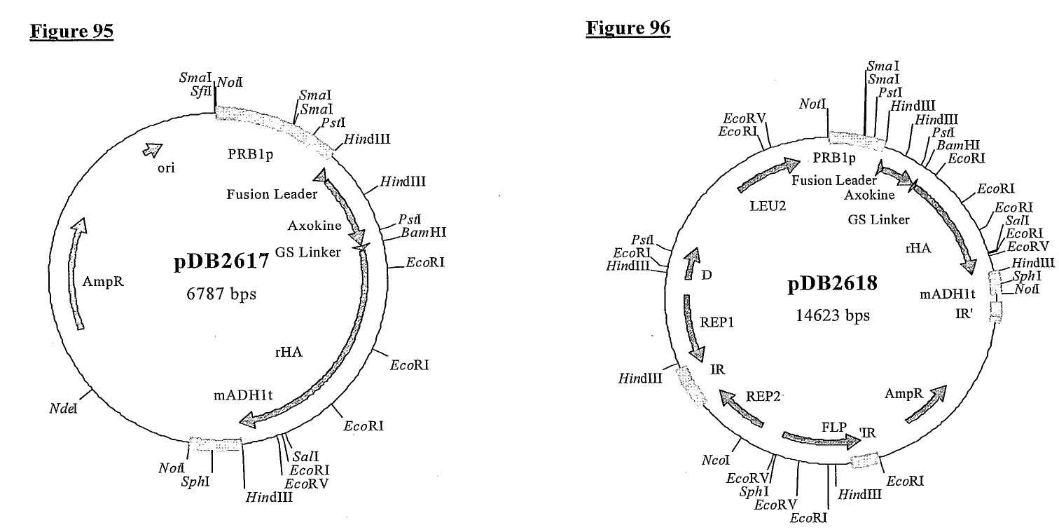

The 0.57 kb BamHI and BglII digested PCR product was ligated with pDB2573X, which had been digested with BamHI, BglII and calf intestinal alkaline phosphatase to create plasmid pDB2617 (FIG. 95) and the correct DNA sequence confirmed for the PCR generated fragment and adjacent sequences using oligonucleotide primers CF84, CF85, PRB and DS229.

CF84
5′-CCTATGTGAAGCATCAGGGC-3′
CF85
5′-CCAACATTAATAGGCATCCC-3′
PRB
5′-CGTCCCGTTATATTGGAG-3′
DS229
5′-CTTGTCACAGTTTTCAGCAGATTCGTCAG-3′

Plasmid pDB2617 was digested with NdeI and NotI, and the 3.586-kb NotI expression cassette for Axokine™-(GGS)₄GG-albumin secretion was purified from an agarose gel.

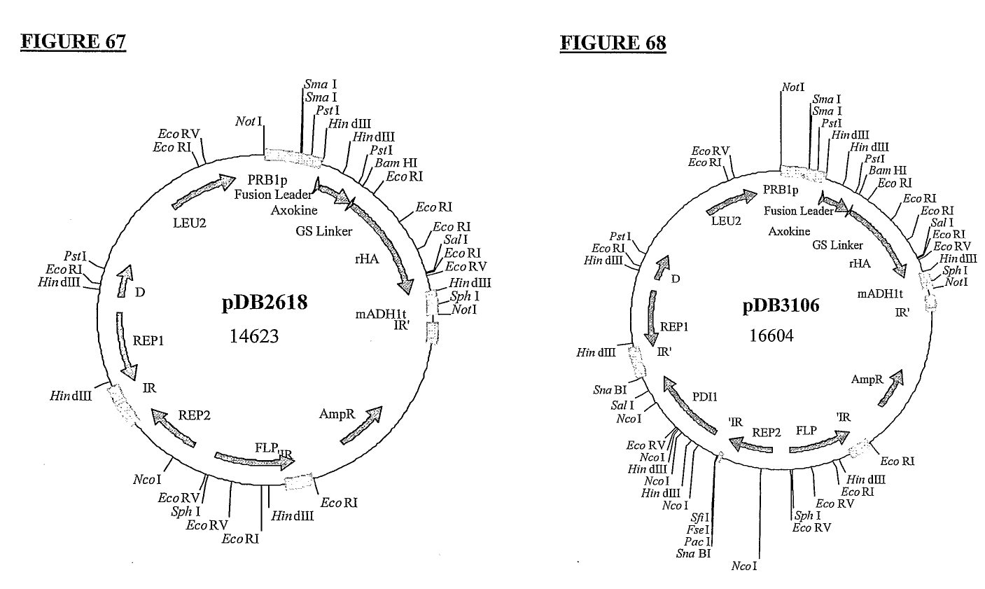

Appropriate yeast vector sequences were provided by the “disintegration” plasmid pSAC35. The 3.586 kb NotI N-terminal Axokine™-(GGS)₄GG-albumin expression cassette was isolated from pDB2617, purified and ligated into NotI digested pSAC35, which had been treated with calf intestinal phosphatase, creating plasmid pDB2618 containing the NotI expression cassette in the same orientation to the LEU2 selection marker (FIG. 96). Appropriate yeast PDI1 vector sequences were provide by a “disintegration” plasmid pDB2690 (FIG. 6). The 3.586 kb NotI N-terminal Axokine™-(GGS)₄GG-albumin expression cassette was isolated from pDB2617, purified and ligated into NotI digested pDB2690, which had been treated with calf intestinal phosphatase, creating plasmid pDB31Q6 containing the NotI expression cassette in the same orientation to the LEU2 selection marker (FIG. 68).

A human IL10 cDNA (NCBI accession number (NM_{—}000572) was amplified by PCR using single stranded oligonucleotides CF68 and CF69.

CF68
5′-GCGCAGATCTTTGGATAAGAGAAGCCCAGGCCAGGGCACCCAGTCTG
AGAACAGCTGCAC-3′
CF69
5′-GCTTGGATCCACCGTTTCGTATCTTCATTGTCATGTAGGCTTCTATG
TAG-3′

The 0.43 kb DNA fragment was digested to completion with BamHI and partially digested with BglII and the 0.42 kb BglII-BamHI DNA fragment isolated.

Plasmid pDB2573X provided a suitable transcription promoter and terminator, along with a suitable secretory leader sequence and DNA sequences encoding part of a (GGS)₄GG peptide linker fused to the N-terminus of human albumin. The construction of pDB2573X has been previously described (WO 03/066824).

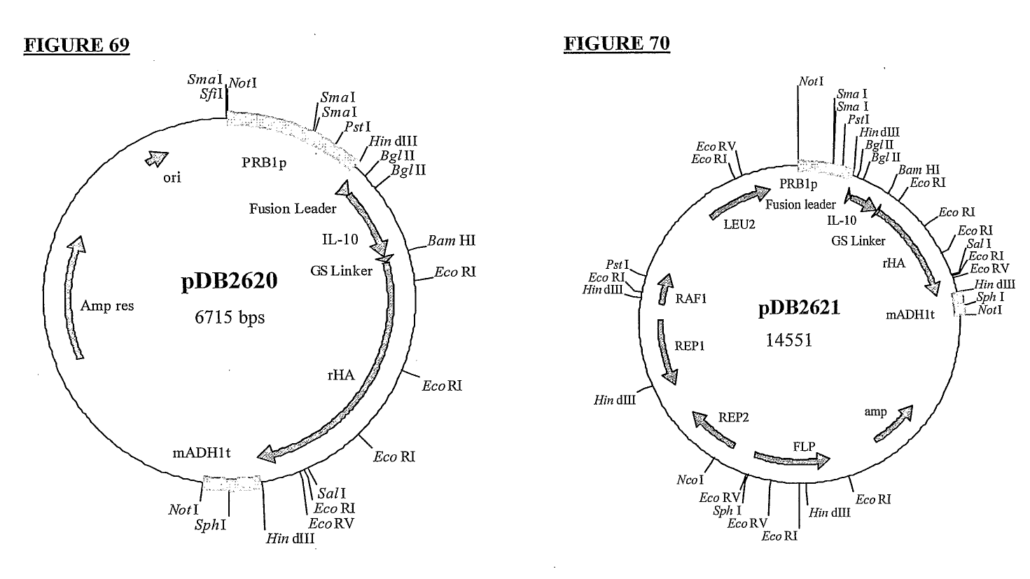

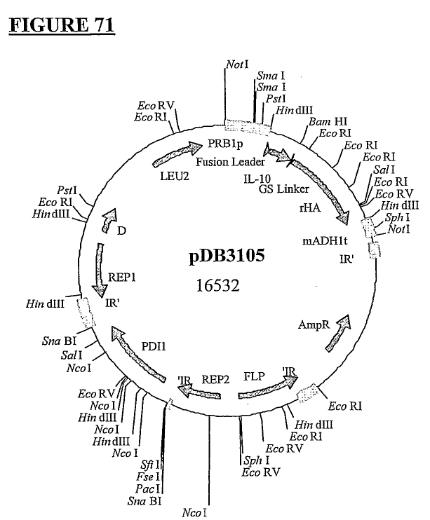

Plasmid pDB2573X was digested to completion with BglII and BamHI, the 6.21 kb DNA fragment was isolated and treated with calf intestinal phosphatase and then ligated with the 0.42 kb BglII/BamHI N-terminal IL10 cDNA to create pDB2620 (FIG. 69). Appropriate yeast vector sequences were provided by the “disintegration” plasmid pSAC35. The 3.511 kb NotI N-terminal IL10-(GGS)₄GG-albumin expression cassette was isolated from pDB2620, purified and ligated into NotI digested pSAC35, which had been treated with calf intestinal phosphatase, creating plasmid pDB2621 containing the NotI expression cassette in the same orientation to the LEU2 selection marker (FIG. 70). An appropriate yeast PDI1 vector sequences were provide by a “disintegration” plasmid pDB2690 (FIG. 6). The 3.51 kb NotI N-terminal IL10-(GGS)₄GG-albumin expression cassette was isolated from pDB2620, purified and ligated into NotI digested pDB2690, which had been treated with calf intestinal phosphatase, creating plasmid pDB3105 containing the NotI expression cassette in the same orientation to the LEU2 selection marker (FIG. 71).

The same control yeast strain as used in previous examples was transformed to leucine prototrophy using a modified lithium acetate method (Sigma yeast transformation kit, YEAST-1, protocol 2; (Ito et al, 1983, J. Bacteriol., 153, 163; Elble, 1992, Biotechniques, 13, 18)). Transformants were selected on BMMD-agar plates, and were subsequently patched out on BMMD-agar plates. Cryopreserved trehalose stocks were prepared from 10 mL BMMD shake flask cultures (24 hrs, 30° C., 200 rpm).

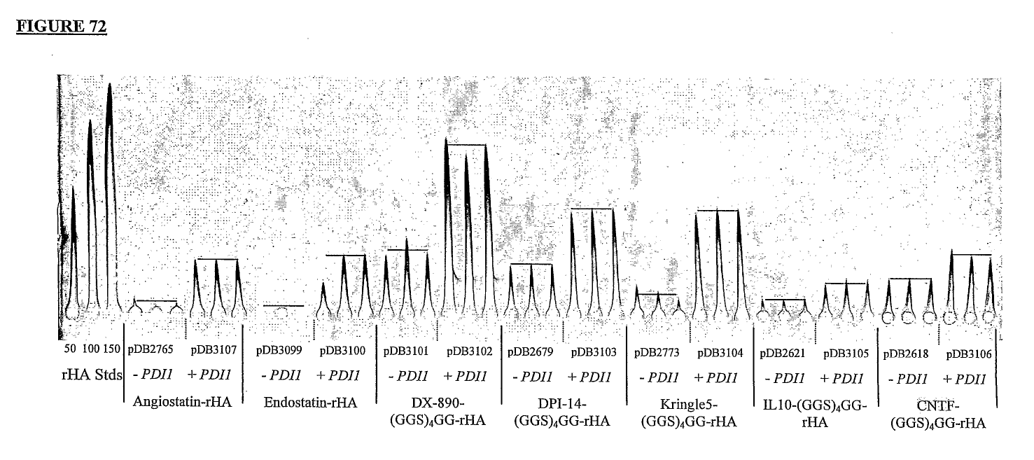

Transformants of each strain were inoculated into 10 mL BMMD in 50 mL shake flasks and incubated in an orbital shaker at 30° C., 200 rpm for 4-days. Culture supernatants were harvested and the recombinant albumin fusion titres compared by rocket immunoelectrophoresis (FIG. 72). The results indicated that the albumin fusion titre in the culture supernatants from yeast strain was higher when PDI1 was present in the 2 μm plasmid than when it was not.

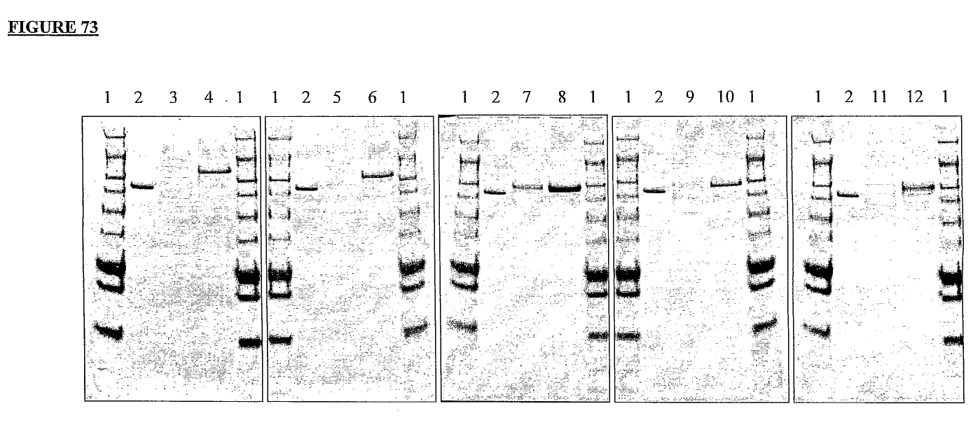

The increase in expression of the albumin fusions detected by rocket immunoelectrophoresis was further studied by SDS-PAGE analysis. BMMD shake flask cultures of YBX7 expressing various albumin-fusions were grown for 4-days in an orbital shaker at 30° C., 200 rpm. A sample of the culture supernatant was analysed by SDS-PAGE (FIG. 73). A protein band of the expected size for the albumin fusion under study was observed increase in abundance.

### Example 10

### Co-Expression of S. cerevisiae ORM2 and Recombinant Transferrin on a 2 μm-Based Plasmid

The ORM2 gene from S. cerevisiae S288c was cloned into the XcmI-site after REP2 on a pSAC35-based plasmid containing an expression cassette for rTf (N413Q, N611Q) at the NotII-site in the UL-region.

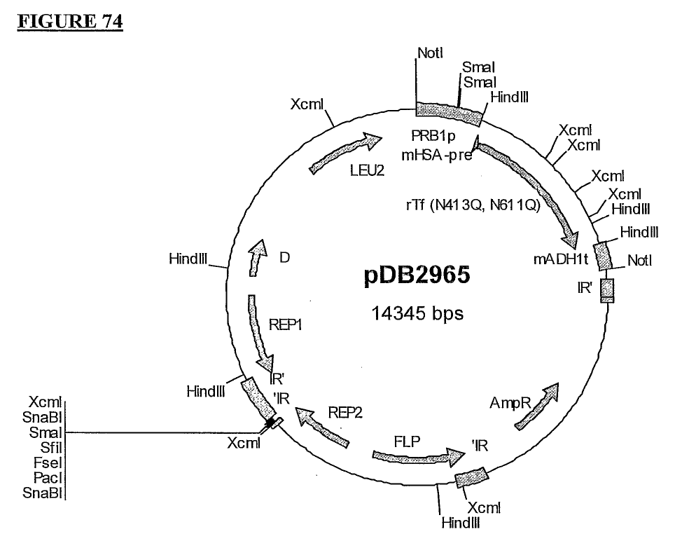

Plasmid pDB2965 (FIG. 74) was constructed by insertion of the 3,256-bp NotI fragment containing the rTf (N413Q, N611Q) expression cassette from pDB2928 (FIG. 11) into the NotI-site of pDB2688 (FIG. 4). pDB2688 was linearised by NotI digestion and was treated with alkaline phosphatase. The rTf expression cassette from pDB2928 was cloned into the NotI site of pDB2688 to produce pDB2965, with the transferrin gene transcribed in the same direction as LEU2.

The ORM2 gene was amplified from S. cerevisiae S288c genomic DNA by PCR with oligonucleotide primers GS11 and GS12 (Table 10) using the Expand High Fidelity ^{PLUS} PCR System (Roche).

TABLE 10

Oligonucleotide Primers for PCR Amplification
of S. cerevisiae Chaperones

| | | |
|---|---|---|
| Primer | Description | Oligonucleotide Sequence |

| | | |
|---|---|---|
| GS11 | ORM2 primer, | 5′-GCGCTACGTATTAATTAAATTGCTCATA |
| | 54 mer | TATAGTGGGGGGGAATACTCATGCTG-3′ |
| GS12 | ORM2 primer, | 5′-GCGCTACGTAGGCCGGCCAGAGAATATA |
| | 49 mer | AAGAAAGATGATGATGTAAGG-3′ |
| CED037 | SSAI primer, | 5′-ATACGCGCATGCGAATAATTTTTTTTTG |
| | 70 mer | CCTATCTATAAAATTAAAGTAGCAGTACTTC |
| | | AACCATTAGTG-3′ |
| CED038 | SSAI primer, | 5′-ATACGCGCATGCCGACAAATTGTTACGT |
| | 50 mer | TGTGCTTTGATTTCTAAAGCGC-3′ |
| CED009 | PSEI primer, | 5′-ATAGCGGGATCCAAGCTTCGACACATAC |
| | 50 mer | ATAATAACTCGATAAGGTATGG-3′ |
| CED010 | PSEI primer, | 5′-TATCGCGGATCCCGTCTTCACTGTACAT |
| | 39 mer | TACACATAAGC-3′ |

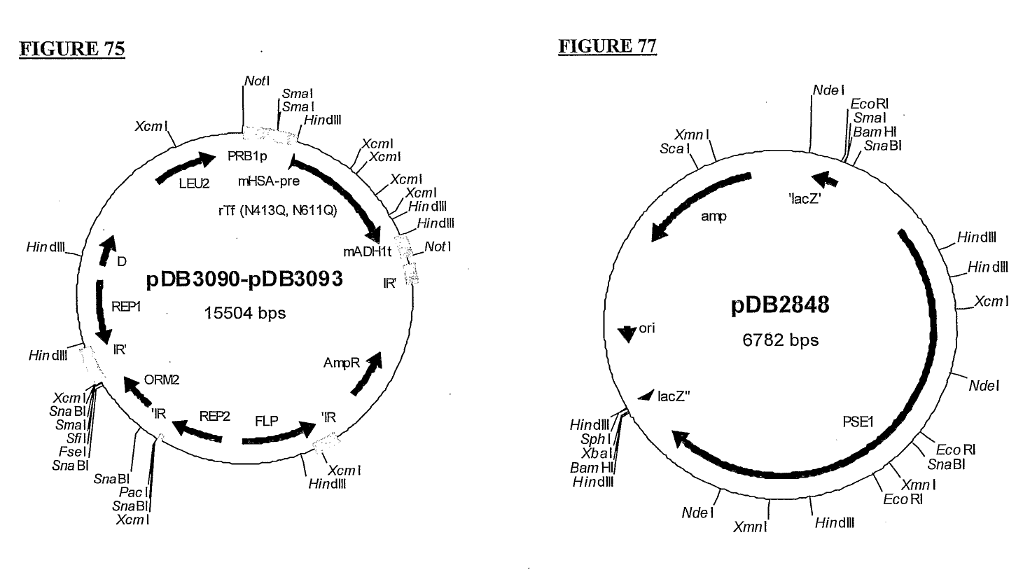

Primers were designed to incorporate SnaBI and PacI restriction recognition sites at the 5′ end of the forward primer and SnaBI and FseI restriction recognition sites at the 5′ end of the reverse primer for cloning into the linker at the XcmI-site of the vector, pDB2965. PCR was carried out under the following conditions: 200 μM dNTP mix, 2.5 U of Expand HiFi enzyme blend, 1× Expand HiFi reaction buffer, 0.8 μg genomic DNA; 1 cycle of 94° C. for 2 minutes, 30 cycles of 94° C. for 30 seconds, 55° C. for 30 seconds, 72° C. for 3 minutes, and 1 cycle 72° C. for 7 minutes. 0.4 μM of each primer was used. The required 1,195-bp PCR product and the pDB2965 vector were digested with PacI and FseI, ligated together and transformed into competent E. coli DH5α, cells. Ampicillin resistant transformants were selected. ORM2-containing constructs were identified by restriction enzyme analysis of plasmid DNA isolated from the ampicillin resistant clones. Four plasmid clones were prepared pDB3090, pDB3091, pDB3092, and pBD3093, all of which had the same expected DNA fragment pattern during restriction analysis (FIG. 75).

The S. cerevisiae Control Strain and Strain A (as described in Example 3) were selected to investigate the effect on transferrin secretion when the transferrin and ORM2 genes were co-expressed from the 2 μm-based plasmids. The Control Strain and Strain A were transformed to leucine prototrophy by plasmids pDB3090, pDB3092 and pBD3093, as well as a control plasmid pDB2931 (FIG. 14), containing the rTf (N413Q, N611Q) expression cassette without ORM2. Transformants were selected on BMMD agar and patched out on BMMD agar for subsequent analysis.

To investigate the effect of ORM2 co-expression on transferrin secretion, 10 mL selective (BMMD) and non-selective (YEPD) liquid media were inoculated with strains containing the ORM2/transferrin co-expression plasmids. The shake flask culture was then incubated at 30° C. with shaking (200 rpm) for 4 days. The relative level of transferrin secretion was determined by rocket gel immunoelectrophoresis (RIE) (FIG. 76).

Levels of transferrin secreted from Control Strain [pDB3090] and Control Strain [pDB3092] were greater than the levels from Control Strain [pDB2931] in both BMMD and YEPS media. Similarly, the levels of transferrin secreted from both Strain A [pDB3090] and Strain A [pDB3093] were greater than the levels from Strain A [pDB2931] in both BMMD and YEPS media. Transferrin secretion from all Strain A transformants was higher than the Control Strain transformants grown in the same media. Strain A contains an additional copy of PDI1 in the genome, which enhanced transferrin secretion. Therefore in Strain A, the increased expression of ORM2 and PDI1 had a cumulative effect on the secretion of transferrin.

### Example 11

### Co-Expression of S. cerevisiae PSE1 and Recombinant Transferrin on a 2 μm-Based Plasmid

The PSE1 gene from S. cerevisiae S288c was cloned into the XcmI-site after REP2 on a pSAC35-based plasmid containing an expression cassette for rTf (N413Q, N611Q) at the NotI-site in the UL-region.

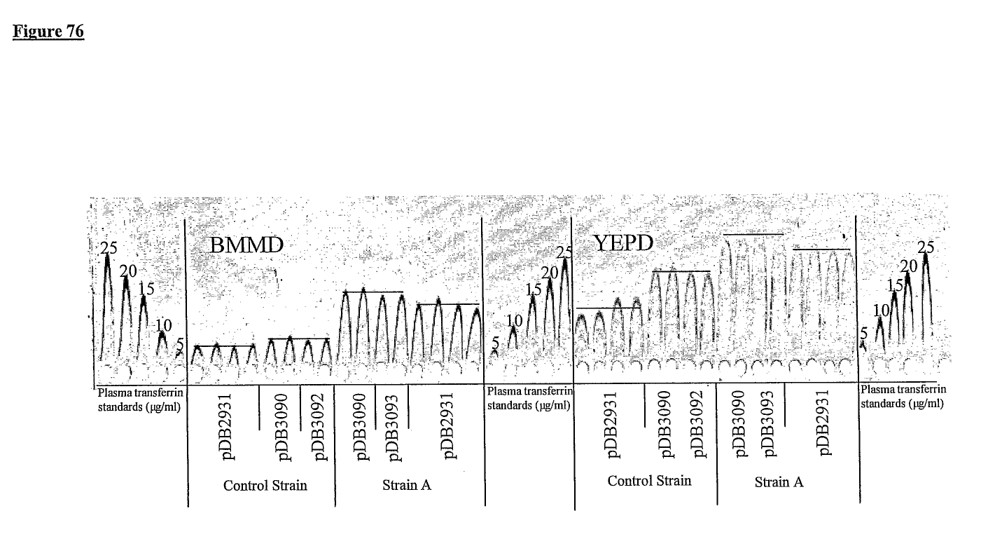

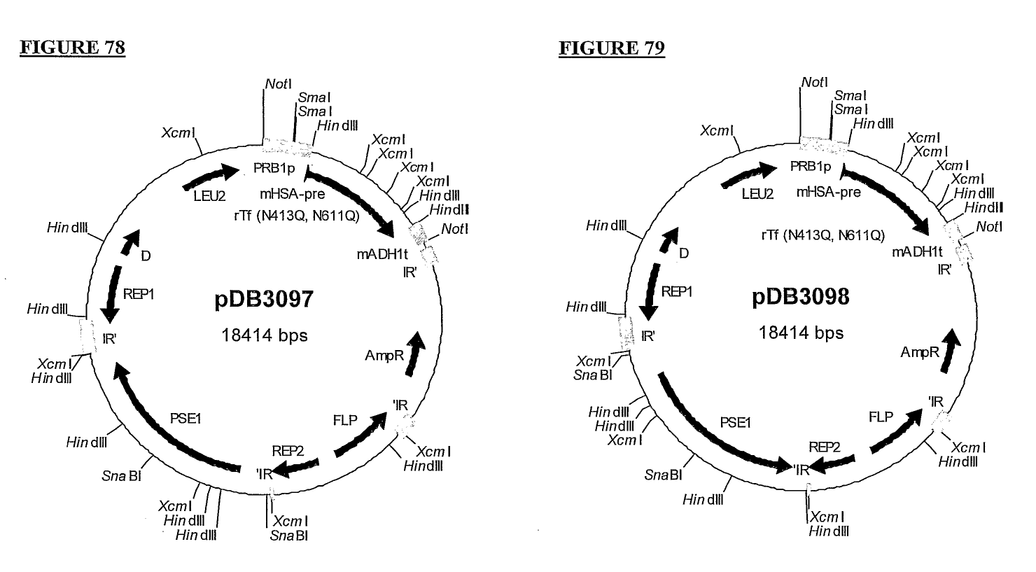

The 3.25-kp wild-type PSE1 gene was amplified from S. cerevisiae S288c genomic DNA by PCR with oligonucleotide primers CED009 and CED010 (Table 10) using the Expand High Fidelity PCR Kit (Roche). Primers were designed to incorporate BamHI restriction recognition sites at the 5′ end to facilitate cloning into the vector, pUC19. PCR was carried out under the following conditions: 1 cycle of 94° C. for 2 minutes; 10 cycles of 94° C. for 15 seconds, 45° C. for 30 seconds, 68° C. for 4 minutes and 30 seconds; 20 cycles of 94° C. for 15 seconds, 45° C. for 30 seconds, 68° C. for 4 minutes and 30 seconds (increasing 5 seconds per cycle); and 1 cycle of 68° C. for 10 minutes. The required PCR product was digested with BamHI then ligated into pUC19, which had been digested with BamHI and treated with alkaline phosphatase, producing construct pDB2848 (FIG. 77). Sequencing of pDB2848 confirmed that amplified sequences were as expected for S. cerevisiae S288c PSE1, when compared to the sequence from PSE1/YMR308c on chromosome XIII from coordinates 892220 to 888951 plus 1000 basepairs of upstream sequence and 1000 basepairs of downstream sequence (Saccharomyces Genome Database at http://www.yeastgenome.org/). The PSE1 gene was then excised from pDB2848 by BamHI digestion, and the resulting 4,096-bp fragment phenol:chloroform extracted, ethanol precipitated and treated with DNA polymerase Klenow fragment to fill in the 5′-overhang. Plasmid pDB2965 (FIG. 74) was linearised by SnaBI digestion, and alkaline phosphatase treated. The linearised pDB2965 vector and the PSE1 insert were ligated, and transformed into competent E. coli DH5α, cells. Ampicillin resistant transformants were selected. Plasmids pDB3097 (FIG. 78) and pDB3098 (FIG. 79) were identified to contain the PSE1 gene by restriction enzyme analysis of plasmid DNA isolated from the ampicillin resistant clones. In pDB3097 the PSE1 gene is transcribed in the same orientation as REP2, whereas in pDB3097 the PSE1 gene is transcribed in the opposite orientation to REP2.

The S. cerevisiae Control Strain was transformed to leucine prototrophy by plasmids, pDB3097 and pBD3098, as well as a control plasmid pDB2931 (FIG. 14), containing the rTf (N413Q, N611Q) expression cassette without PSE1. Transformants were selected on BMMD agar and patched out on BMMD agar for subsequent analysis.

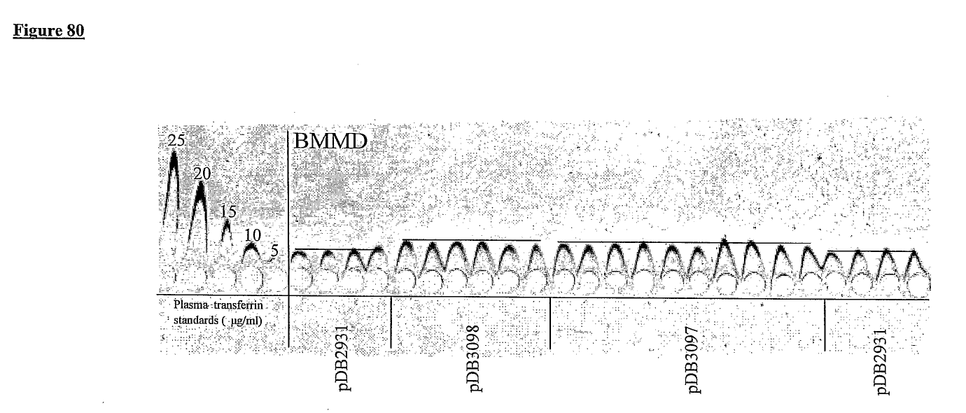

To investigate the effect of PSE1 expression on transferrin secretion, flasks containing 10 mL selective (BMMD) liquid media were inoculated with strains containing the PSE1/transferrin co-expression plasmids. The shake flask culture was then incubated at 30° C. with shaking (200 rpm) for 4 days. The relative level of transferrin secretion was determined by rocket gel immunoeletrophoresis (RIE) (FIG. 80).

Levels of transferrin secreted from Control Strain [pDB3097] and Control Strain [pDB3098] were greater than the levels from Control Strain [pDB2931] in BMMD media. Therefore, expression of PSE1 from the 2 μm-based plasmids had enhanced transferrin secretion from S. cerevisiae. Transferrin secretion was improved with the PSE1 gene transcribed in either direction relative to the REP2 gene in pDB3097 and pDB3098.

### Example 12

### Co-Expression of S. cerevisiae SSA1 and Recombinant Transferrin on a 21 on-Based Plasmid

The SSA1 gene from S. cerevisiae S288c was cloned into the XcmI-site after REP2 on a pSAC35-based plasmid containing an expression cassette for rTf (N413Q, N611Q) at the NotI-site in the UL-region.

The 1.93-kb SSA1 gene was amplified from S. cerevisiae S288c genomic DNA by PCR with oligonucleotide primers CED037 and CED038 (Table 10) using the Expand High Fidelity PCR Kit (Roche). Primers were designed to incorporate SphI restriction recognition sites at their 5′ ends to facilitate cloning into the vector, pUC19. PCR was carried out under the following conditions: 1 cycle of 94° C. for 10 minutes, 35 cycles of 94° C. for 1 minute, 55° C. for 1 minute, 72° C. for 5 minutes, and 1 cycle of 72° C. for 10 minutes. The required PCR product was digested with SphI then ligated into pUC19, which had been digested with SphI and treated with alkaline phosphatase, producing construct pDB2850 (FIG. 81). Sequencing of pDB2850 confirmed the expected sequence of S. cerevisiae S288c SSA1/YAL005C on chromosome I from coordinates 141433 to 139505 plus 1000 basepairs of upstream sequence and 1000 basepairs of downstream published in the Saccharomyces Genome Database (http://www.yeastgenome.org/).

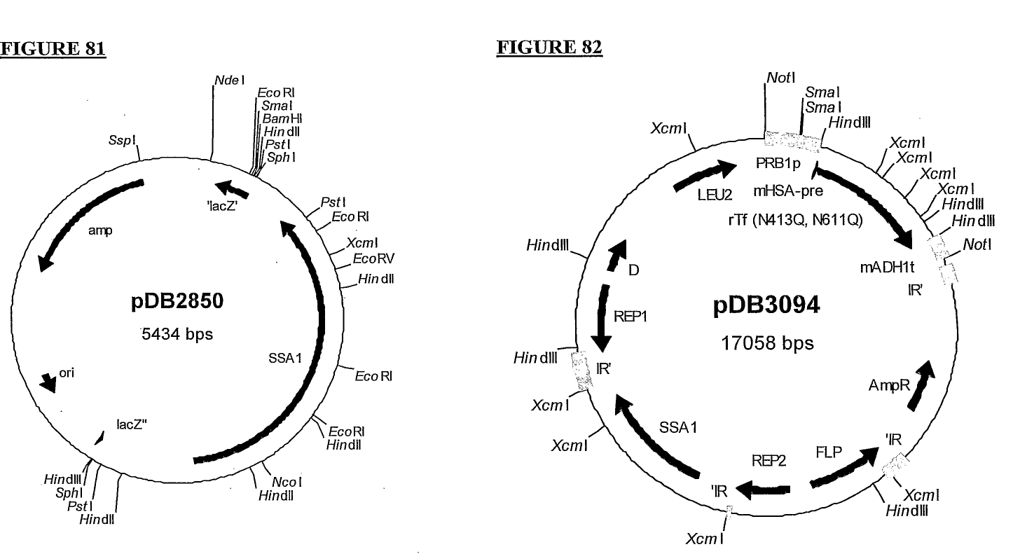

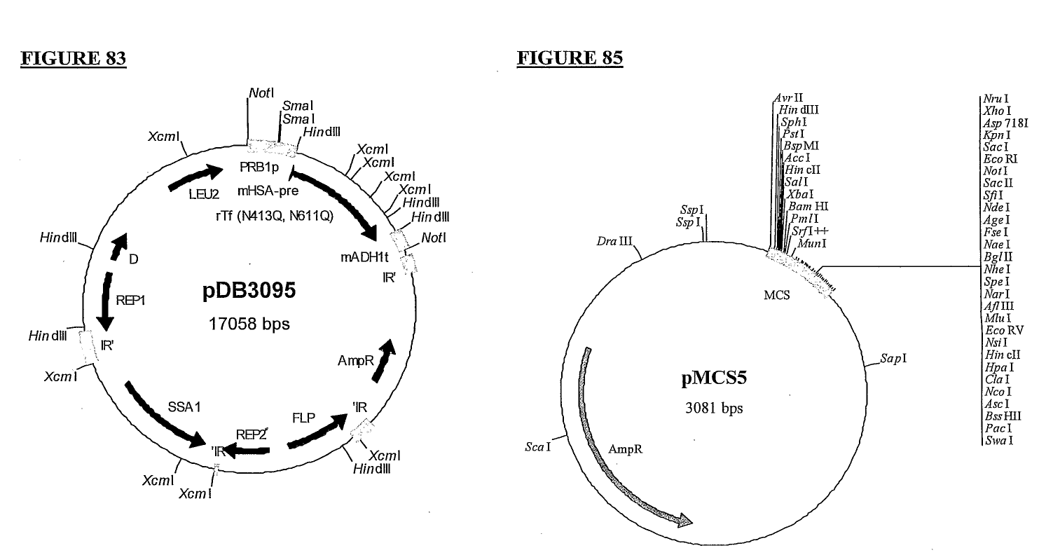

The SSA1 gene was excised from pDB2850 by SphI-digestion, and the resulting 2,748-bp fragment phenol:chloroform extracted, ethanol precipitated and treated with T4 DNA polymerase to remove the 3′-overhang. Plasmid pDB2965 was linearised by SnaBI digestion and treated with calf alkaline phosphatase. The linearised pDB2965 vector and the SSA1 insert were ligated and transformed into competent E. coli DH5α cells. Ampicillin resistant transformants were selected. SSA1 constructs pDB3094 (FIG. 82), and pDB3095 (FIG. 83) were identified by restriction enzyme analysis of plasmid DNA isolated from the ampicillin resistant clones. In pDB3094, the SSA1 gene is transcribed in the same direction as REP2, whereas in pDB3095 the SSA1 gene is transcribed in the opposite direction to REP2.

The S. cerevisiae Control Strain was transformed to leucine prototrophy by plasmids, pDB3094 and pBD3095, as well as a control plasmid pDB2931 (FIG. 14), containing the rTf (N413Q, N611Q) expression cassette without SSA1. Transformants were selected on BMMD agar and patched out on BMMD agar for subsequent analysis.

To investigate the effect of SSA1 expression on transferrin secretion, flasks containing 10 mL selective (BMMD) liquid media were inoculated with strains containing the SSA1/transferrin co-expression plasmids. The shale flask cultures were incubated at 30° C. with shaking (200 rpm) for 4 days. The relative level of transferrin secretion was determined by rocket gel immunoeletrophoresis (RIE) (FIG. 84).

Levels of transferrin secreted from Control Strain [pDB3095] were greater than the levels from Control Strain [pDB2931] and Control Strain [pDB3094] in BMMD media. Therefore, expression of SSA1 from the 2 μm-based plasmids had enhanced transferrin secretion from S. cerevisiae. Transferrin secretion was improved with the SSA1 gene transcribed in the opposite direction relative to the REP2 gene in pDB3094.

### Example 13

### PDI1 Gene Disruption, Combined with a PDI1 Gene on the 2 μm-Based Plasmid Enhanced the Secretion of Recombinant Albumin and Plasmid Stability

Single stranded oligonucleotide DNA primers listed in Table 11 were designed to amplify a region upstream of the yeast PDI1 coding region and another a region downstream of the yeast PDI1 coding region.

TABLE 11
Oligonucleotide primers

| | | |
|---|---|---|
| Primer | Description | Sequence |

| | | |
|---|---|---|
| DS299 | 5′ PDII | 5′-CGTAGCGGCCGCCTGAA |
| | primer, | AGGGGTTGACCGTCCGTCGG |
| | 38 mer | C-3′ |
| DS300 | 5′ PDII | 5′-CGTAAAGCTTGGCCGCC |
| | primer, | CGACAGGGTAACATATTATC |
| | 40 mer | AC-3′ |
| DS301 | 3′ PDII | 5′-CGTAAAGCTTGACCACG |
| | primer, | TAGTAATAATAAGTGCATGG |
| | 38 mer | C-3′ |
| DS302 | 3′ PDII | 5′-CGTACTGCAGATTGGAT |
| | primer, | AGTGATTAGAGTGTATAGTC |
| | 41 mer | CCGG-3′ |
| DS303 | 18 mer | 5′-GGAGCGACAAACCTTTC |
| | | G-3′ |
| DS304 | 20 mer | 5′-AGCGTAATAAAAGATGG |
| | | CTG-3′ |
| DS305 | 24 mer | 5′-CATCTTGTGTGTGAGTA |
| | | TGGTCGG-3′ |
| DS306 | 14 mer | 5′-CCCAGGATAATTTTCAG |
| | | G-3′ |

Primers DS299 and DS300 amplified the 5′ region of PDI1 by PCR, while primers DS301 and DS302 amplified a region 3′ of PDI1, using genomic DNA derived S288c as a template. The PCR conditions were as follows: 1 μL S288c template DNA (at 0.01 ng/μL, 0.1 ng/μL, 1 ng/μL, 10 ng/μL and 100 ng/μL), 5 μL 10× Buffer (Fast Start Taq+Mg, (Roche)), 1 μL 10 mM dNTP's, 5 μL each primer (2 μM), 0.4 μL Fast Start Taq, made up to 50 μL with H₂O. PCRs were performed using a Perkin-Elmer Thermal Cycler 9700. The conditions were: denature at 95° C. for 4 min [HOLD], then [CYCLE] denature at 95° C. for 30 seconds, anneal at 45° C. for 30 seconds, extend at 72° C. for 45 seconds for 20 cycles, then [HOLD] 72° C. for 10 min and then [HOLD] 4° C. The 0.22 kbp PDI1 5′ PCR product was cut with NotI and HindIII, while the 0.34 kbp PDI1 3′ PCR product was cut with HindIII and PstI.

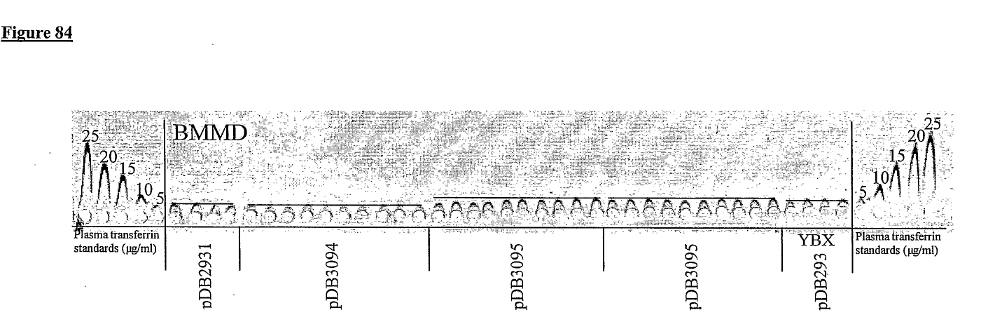

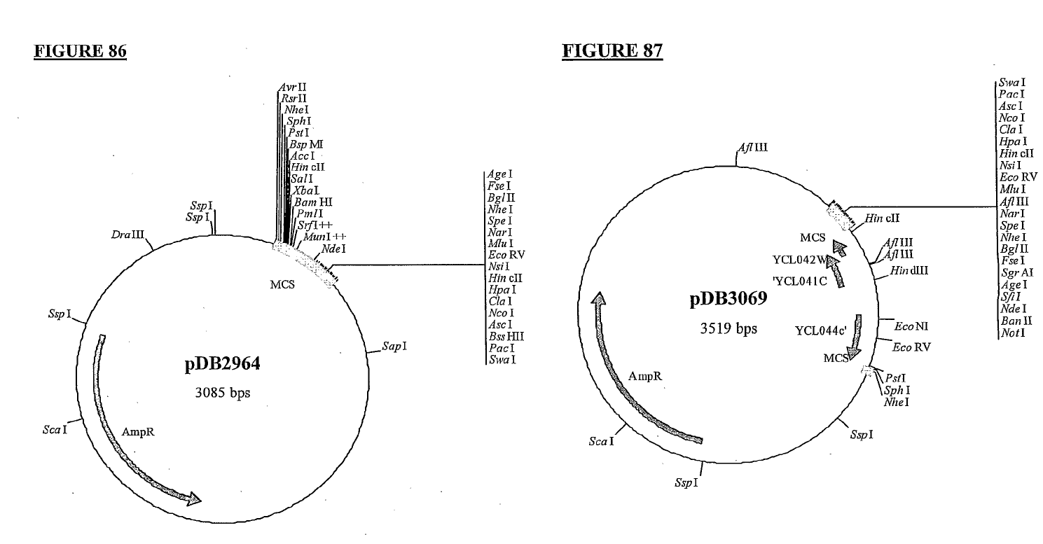

Plasmid pMCS5 (Hoheisel, 1994, Biotechniques 17, 456-460) (FIG. 85) was digested to completion with HindIII, blunt ended with T4 DNA polymerase plus dNTPs and religated to create pDB2964 (FIG. 86).

Plasmid pDB2964 was HindIII digested, treated with calf intestinal phosphatase, and ligated with the 0.22 kbp PDI1 5′ PCR product digested with NotI and HindIII and the 0.34 kbp PDI1 3′ PCR product digested with HindIII and PstI to create pDB3069 (FIG. 87) which was sequenced with forward and reverse universal primers and the DNA sequencing primers DS303, DS304, DS305 and DS306 (Table 11).

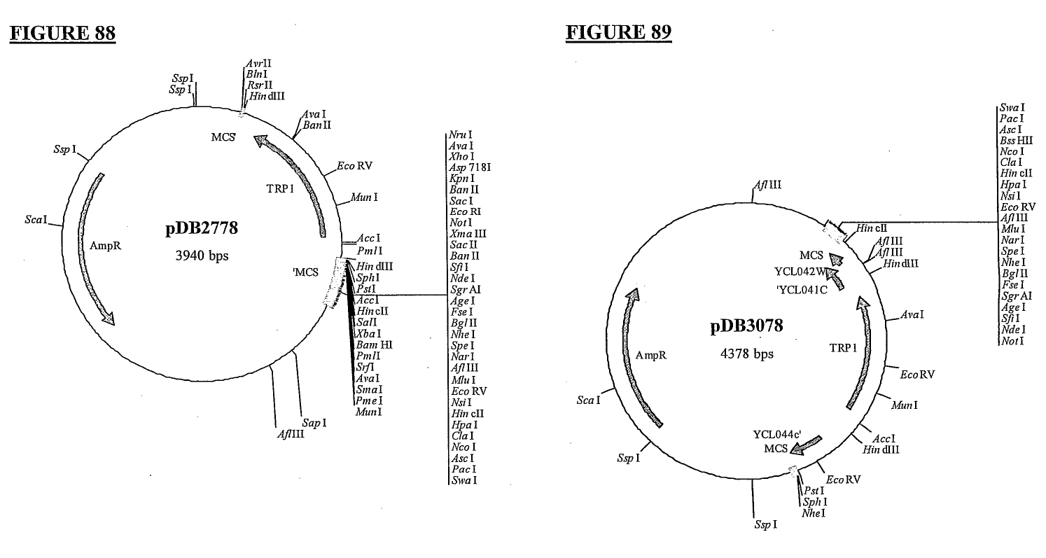

Primers DS234 and DS235 (Table 12) were used to amplify the modified TRP1 marker gene from YIplac204 (Gietz & Sugino, 1988, Gene, 74, 527-534), incorporating HindIII restriction sites at either end of the PCR product. The PCR conditions were as follows: 1 L template YIplac204 (at 0.01 ng/μL, 0.1 ng/μL, 1 ng/μL, 10 ng/μL and 100 ng/μL), 5 μL 10× Buffer (Fast Start Taq+Mg, (Roche)), 1 μL 10 mM dNTP's, 5 μL each primer (2 μM), 0.4 μL Fast Start Taq, made up to 50 μL with H₂O. PCRs were performed using a Perkin-Elmer Thermal Cycler 9600. The conditions were: denature at 95° C. for 4 min [HOLD], then [CYCLE] denature at 95° C. for 30 seconds, anneal for 45 seconds at 45° C., extend at 72° C. for 90 sec for 20 cycles, then [HOLD] 72° C. for 10 min and then [HOLD] 4° C. The 0.86 kbp PCR product was digested with HindIII and cloned into the HindIII site of pMCS5 to create pDB2778 (FIG. 88). Restriction enzyme digestions and sequencing with universal forward and reverse primers as well as DS236, DS237, DS238 and DS239 (Table 12) confirmed that the sequence of the modified TRP1 gene was correct.

TABLE 12
Oligonucleotide primers

| | | |
|---|---|---|
| Primer | Description | Sequence |

| | | |
|---|---|---|
| DS230 | TRP1 5′ UTR | 5′-TAGCGAATTC AATCAGTAAAAATCAACG |
| | | G-3′ |
| DS231 | TRP1 5′ UTR | 5′-GTCAAAGCTTCAAAAAAAGA AAAGCTCC |
| | | GG-3′ |
| DS232 | TRP1 3′ UTR | 5′-TAGCGGATCGGAATTCGGCGGTTGTTTGC |
| | | AAGACCGAG-3′ |
| DS233 | TRP1 3′ UTR | 5′-GTCAAAGCTTTAAAGATAATGCTAAATCA |
| | | TTTGG-3′ |
| DS234 | TRP1 | 5′-TGACAAGCTTTCGGTCGAAAAAAGAAAAG |
| | | G AGAGG-3′ |
| DS235 | TRP1 | 5′-TGACAAGCTTGATCTTTTATGCTTGCTTT |
| | | TC-3′ |
| DS236 | TRP1 | 5′-AATAGTTCAGGCACTCCG-3′ |
| DS237 | TRP1 | 5′-TGGAAGGCAAGAGAGCC-3′ |
| DS238 | TRP1 | 5′-TAAAATGTAAGCTCTCGG-3′ |
| DS239 | TRP1 | 5′-CCAACCAAGTATTTCGG-3′ |
| CED005 | ΔTRP1 | 5′-GAGCTGACAGGGAAATGGTC-3′ |
| CED006 | ΔTRP1 | 5′-TACGAGGATACGGAGAGAGG-3′ |

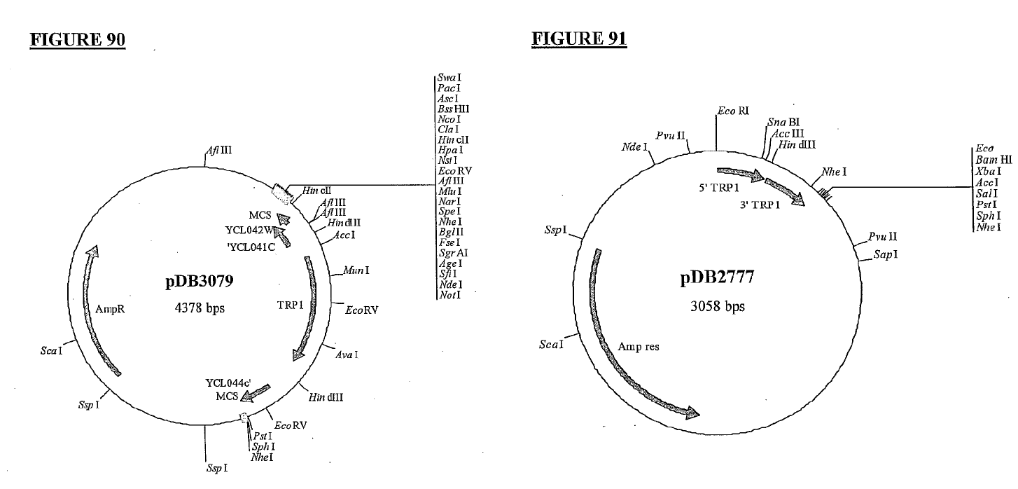

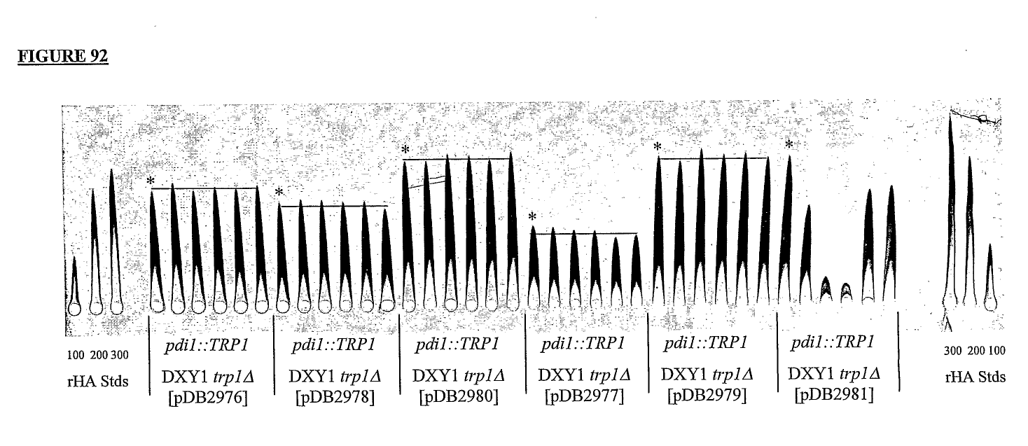

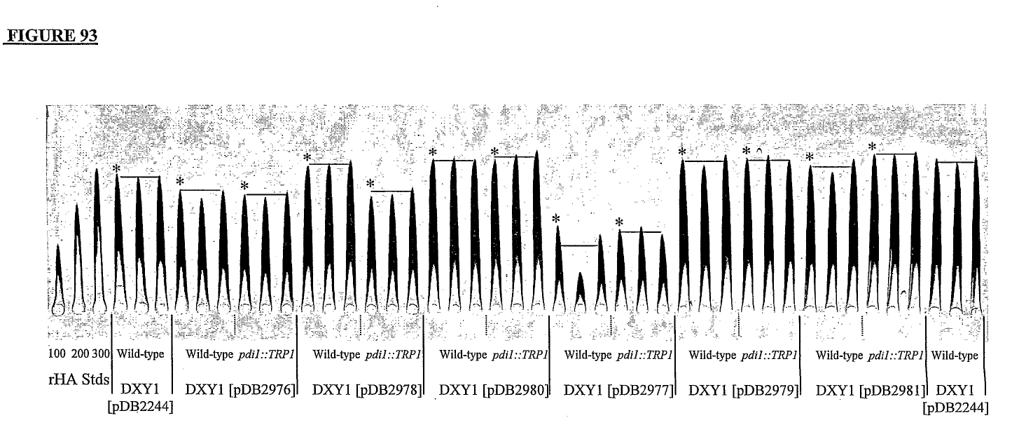

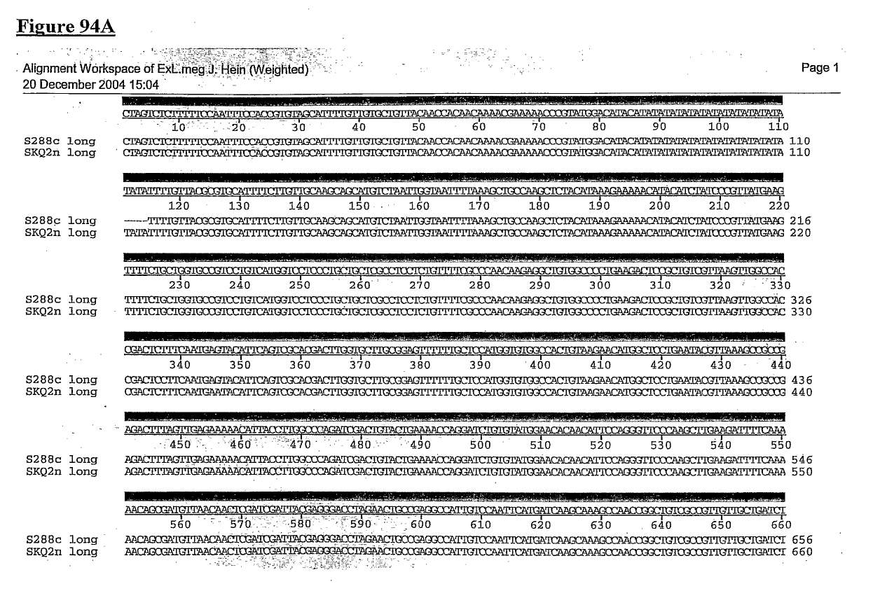

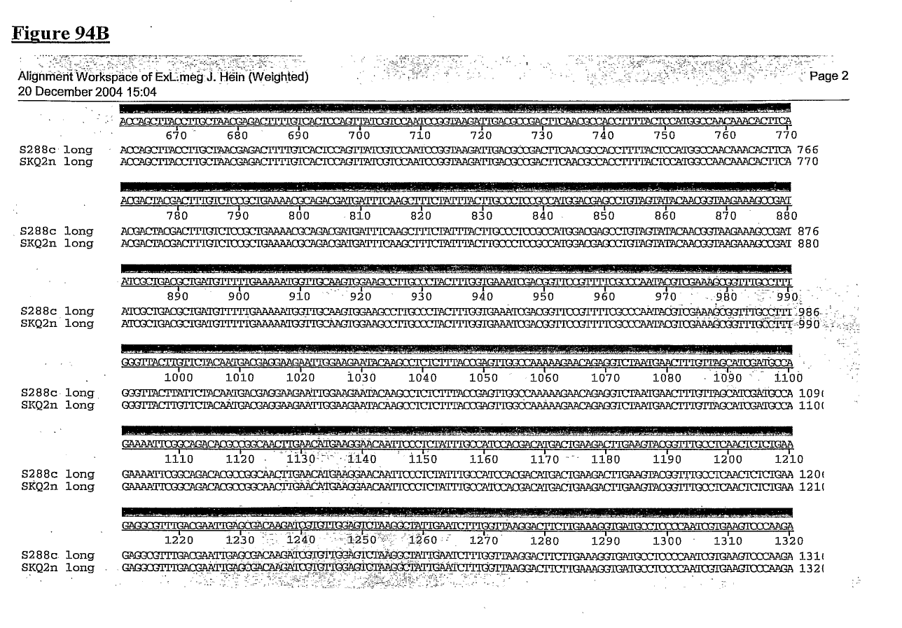

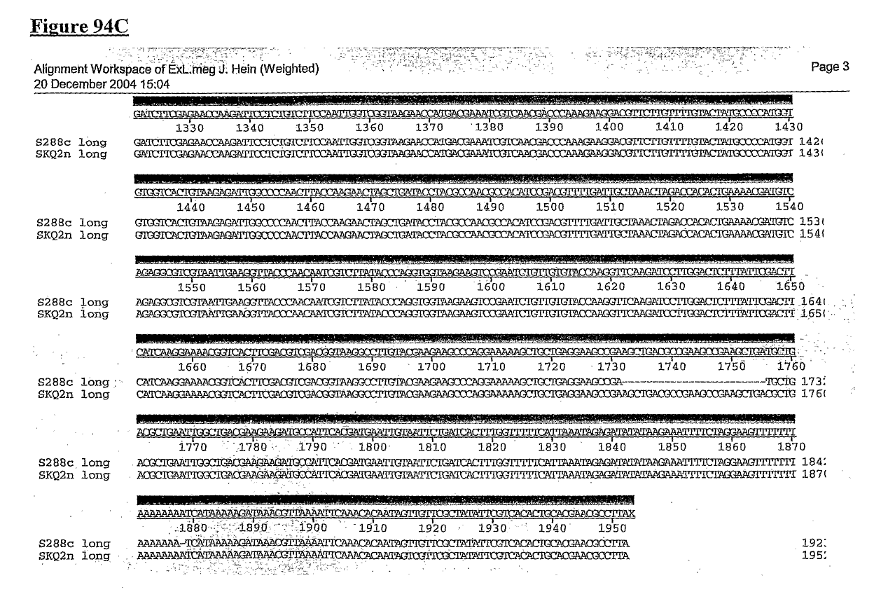

The 0.86 kbp TRP1 gene was isolated from pDB2778 by digestion with HindIII and cloned into the HindIII site of pDB3069 to create pDB3078 (FIG. 89) and pDB3079 (FIG. 90). A 1.41 kb pdi1::TRP1 disrupting DNA fragment was isolated from pDB3078 or pDB3079 by digestion with NotI/PstI.

Yeast strains incorporating a TRP1 deletion (trp1Δ) were to be constructed in such a way that no homology to the TRP1 marker gene (pDB2778) should left in the genome once the trp1Δ had been created, so preventing homologous recombination between future TRP1 containing constructs and the TRP1 locus. In order to achieve the total removal of the native TRP1 sequence from the genome of the chosen host strains, oligonucleotides were designed to amplify areas of the 5′ UTR and 3′ UTR of the TRP1 gene outside of TRP1 marker gene present on integrating vector YIplac204 (Gietz & Sugino, 1988, Gene, 74, 527-534). The YIplac204 TRP1 marker gene differs from the native/chromosomal TRP1 gene in that internal HindIII, PstI and XbaI sites were removed by site directed mutagenesis (Gietz & Sugino, 1988, Gene, 74, 527-534). The YIplac204 modified TRP1 marker gene was constructed from a 1.453 kbp blunt-ended genomic fragment EcoRI fragment, which contained the TRP1 gene and only 102 bp of the TRP1 promoter (Gietz & Sugino, 1988, Gene, 74, 527-534). Although this was a relatively short promoter sequence it was clearly sufficient to complement trp1 auxotrophic mutations (Gietz & Sugino, 1988, Gene, 74, 527-534). Only DNA sequences upstream of the EcoRI site, positioned 102 bp 5′ to the start of the TRP1 ORF were used to create the 5′ TRP1 UTR. The selection of the 37 UTR was less critical as long as it was outside the 3′ end of the functional modified TRP1 marker, which was chosen to be 85 bp downstream of the translation stop codon.

Single stranded oligonucleotide DNA primers were designed and constructed to amplify the 5′ UTR and 3′ UTR regions of the TRP1 gene so that during the PCR amplification restriction enzyme sites would be added to the ends of the PCR products to be used in later cloning steps. Primers DS230 and DS231 (Table 12) amplified the 5′ region of TRP1 by PCR, while primers DS232 and DS233 (Table 12) amplified a region 3′ of TRP1, using S288c genomic DNA as a template. The PCR conditions were as follows: 1 μL template S288c genomic DNA (at 0.01 ng/μL, 0.1 ng/μL, 1 ng/μL, 10 ng/μL and 100 ng/μL), 5 μL 10× Buffer (Fast Start Taq+Mg, (Roche)), 1 μL 10 mM dNTP's, 5 μL each primer (2 μM), 0.4 μL Fast Start Taq, made up to 50 μL with H₂O. PCRs were performed using a Perkin-Elmer Thermal Cycler 9600. The conditions were: denature at 95° C. for 4 min [HOLD], then [CYCLE] denature at 95° C. for 30 seconds, anneal for 45 seconds at 45° C., extend at 72° C. for 90 sec for 20 cycles, then [HOLD] 72° C. for 10 min and then [HOLD] 4° C.

The 0.19 kbp TRP1 5′ UTR PCR product was cut with EcoRI and HindIII, while the 0.2 kbp TRP1 3′ UTR PCR product was cut with BamHI and HindIII and ligated into pAYE505 linearised with BamHI/EcoRI to create plasmid pDB2777 (FIG. 91). The construction of pAYE505 is described in WO 95/33833. DNA sequencing using forward and reverse primers, designed to prime from the plasmid backbone and sequence the cloned inserts, confirmed that in both cases the cloned 5′ and 3′ UTR sequences of the TRP1 gene had the expected DNA sequence. Plasmid pDB2777 contained a TRP1 disrupting fragment that comprised a fusion of sequences derived from the 5′ and 3′ UTRs of TRP1. This 0.383 kbp TRP1 disrupting fragment was excised from pDB2777 by complete digestion with EcoRI.

Yeast strain DXY1 (Kerry-Williams et al., 1998, Yeast, 14, 161-169) was transformed to leucine prototrophy with the albumin expression plasmid pDB2244 using a modified lithium acetate method (Sigma yeast transformation kit, YEAST-1, protocol 2; (Ito et al, 1983, J. Bacteriol., 153, 163; Elble, 1992, Biotechniques, 13, 18)) to create yeast strain DXY1 [pDB2244]. The construction of the albumin expression plasmid pDB2244 is described in WO 00/44772. Transformants were selected on BMMD-agar plates, and were subsequently patched out on BMMD-agar plates. Cryopreserved trehalose stocks were prepared from 10 mL BMMD shake flask cultures (24 hrs, 30° C., 200 rpm).

DXY1 [pDB2244] was transformed to tryptophan autotrophy with the 0.383 kbp EcoRI TRP1 disrupting DNA fragment from pDB2777 using a nutrient agar incorporating the counter selective tryptophan analogue, 5-fluoroanthranilic acid (5-FAA), as described by Toyn et al., (2000 Yeast 16, 553-560). Colonies resistant to the toxic effects of 5-FAA were picked and streaked onto a second round of 5-FAA plates to confirm that they really were resistant to 5-FAA and to select away from any background growth. Those colonies which grew were then were re-patched onto BMMD and BMMD plus tryptophan to identify which were tryptophan auxotrophs.

Subsequently colonies that had been shown to be tryptophan auxotrophs were selected for further analysis by transformation with YCplac22 (Gietz & Sugino, 1988, Gene, 74, 527-534) to ascertain which isolates were trp1.

PCR amplification across the TRP1 locus was used to confirm that the trp⁻ phenotype was due to a deletion in this region. Genomic DNA was prepared from isolates identified as resistant to 5-FAA and unable to grow on minimal media without the addition of tryptophan. PCR amplification of the genomic TRP1 locus with primers CED005 and CED006 (Table 12) was achieved as follows: 1 μL template genomic DNA, 5 μL 10× Buffer (Fast Start Taq+Mg, (Roche)), 1 μL 10 mM dNTP's, 5 μL each primer (2 μM), 0.4 μL Fast Start Taq, made up to 50 μL with H₂O. PCRs were performed using a Perkin-Elmer Thermal Cycler 9600. The conditions were: denature at 94° C. for 10 min [HOLD], then [CYCLE] denature at 94° C. for 30 seconds, anneal for 30 seconds at 55° C., extend at 72° C. for 120 sec for 40 cycles, then [HOLD] 72° C. for 10 min and then [HOLD] 4° C. PCR amplification of the wild type TRP1 locus resulted in a PCR product of 1.34 kbp in size, whereas amplification across the deleted TRP1 region resulted in a PCR product 0.84 kbp smaller at 0.50 kbp. PCR analysis identified a DXY1 derived trp⁻ strain (DXY1 trp1Δ [pDB2244]) as having the expected deletion event.

The yeast strain DXY1 trp1Δ [pDB2244] was cured of the expression plasmid pDB2244 as described by Sleep et al., (1991, Bio/Technology, 9, 183-187). DXY1 trp1Δ cir⁰ was re-transformed the leucine prototrophy with either pDB2244, pDB2976, pDB2977, pDB2978, pDB2979, pDB2980 or pDB2981 using a modified lithium acetate method (Sigma yeast transformation kit, YEAST-1, protocol 2; (Ito et al, 1983, J. Bacteriol., 153, 163; Elble, 1992, Biotechniques, 13, 18)). Transformants were selected on BMMD-agar plates supplemented with tryptophan, and were subsequently patched out on BMMD-agar plates supplemented with tryptophan. Cryopreserved trehalose stocks were prepared from 10 mL BMMD shake flask cultures supplemented with tryptophan (24 hrs, 30° C., 200 rpm).

The yeast strains DXY1 trp1Δ [pDB2976], DXY1 trp1Δ [pDB2977], DXY1 trp1Δ [pDB2978], DXY1 trp1Δ [pDB2979], DXY1 trp1Δ [pDB2980] or DXY1 trp1Δ [pDB2981] was transformed to tryptophan prototrophy using the modified lithium acetate method (Sigma yeast transformation kit, YEAST-1, protocol 2; (Ito et al, 1983, J. Bacteriol., 153, 163; Elble, 1992, Biotechniques, 13, 18)) with a 1.41 kb pdi1::TRP1 disrupting DNA fragment was isolated from pDB3078 by digestion with NotI/PstI. Transformants were selected on BMMD-agar plates and were subsequently patched out on BMMD-agar plates.

Six transformants of each strain were inoculated into 10 mL YEPD in 50 mL shake flasks and incubated in an orbital shaker at 30° C., 200 rpm for 4-days. Culture supernatants and cell biomass were harvested. Genomic DNA was prepared (Lee, 1992, Biotechniques, 12, 677) from the tryptophan prototrophs and DXY1 [pDB2244]. The genomic PDI1 locus amplified by PCR of with primers DS236 and DS303 (Table 11 and 12) was achieved as follows: 1 μL template genomic DNA, 5 μL 10× Buffer (Fast Start Taq+Mg, (Roche)), 1 μL 10 mM dNTP's, 5 μL each primer (2 μM), 0.4 μL Fast Start Taq, made up to 50 μL with H₂O. PCRs were performed using a Perkin-Elmer Thermal Cycler 9700. The conditions were: denature at 94° C. for 4 min [HOLD], then [CYCLE] denature at 94-C for 30 seconds, anneal for 30 seconds at 50° C., extend at 72° C. for 60 sec for 30 cycles, then [HOLD] 72° C. for 10 min and then [HOLD] 4° C. PCR amplification of the wild type PDI1 locus resulted in no PCR product, whereas amplification across the deleted PDI1 region resulted in a PCR product 0.65 kbp. PCR analysis identified that all 36 potential pdi1::TRP1 strains tested had the expected pdi1::TRP1 deletion.

The recombinant albumin titres were compared by rocket immunoelectrophoresis (FIG. 92). Within each group, all six pdi1::TRP1 disruptants of DXY1 trp1Δ [pDB2976], DXY1 trp1Δ [pDB2978], DXY1 trp1Δ [pDB2980], DXY1 trp1Δ [pDB2977] and DXY1 trp1Δ [pDB2979] had very similar rHA productivities. Only the six pdi1::TRP1 disruptants of DXY1 trp1Δ [pDB2981] showed variation in rHA expression titre. The six pdi1::TRP1 disruptants indicated in FIG. 92 were spread onto YEPD agar to isolate single colonies and then re-patched onto BMMD agar.

Three single celled isolates of DXY1 trp1Δ pdi1::TRP1 [pDB2976], DXY1 trp1Δ pdi1::TRP1 [pDB2978], DXY1 trp1Δ pdi1::TRP1 [pDB2980], DXY1 trp1Δ pdi1::TRP1 [pDB2977], DXY1 trp1Δ pdi1::TRP1 [pDB2979] and DXY1 trp1Δ pdi1::TRP1 [pDB2981] along with DXY1 [pDB2244], DXY1 [pDB2976], DXY1 [pDB2978], DXY1 [pDB2980], DXY1 [pDB2977], DXY1 [pDB2979] and DXY1 [pDB2981] were inoculated into 10 mL YEPD in 50 mL shake flasks and incubated in an orbital shaker at 30° C., 200 rpm for 4-days. Culture supernatants were harvested and the recombinant albumin titres were compared by rocket immunoelectrophoresis (FIG. 93). The thirteen wild type PDI1 and pdi1::TRP1 disruptants indicated in FIG. 93 were spread onto YEPD agar to isolate single colonies. One hundred single celled colonies from each strain were then re-patched onto BMMD agar or YEPD agar containing a goat anti-HSA antibody to detect expression of recombinant albumin (Sleep et al., 1991, Bio/Technology, 9, 183-187) and the Leu+/rHA+, Leu+/rHA−, Leu−/rHA+ or Leu−/rHA− phenotype of each colony scored (Table 13).

TABLE 13

| | PDI1 | | | | pdi1::TRP1 | | | |
|---|---|---|---|---|---|---|---|---|
| | Leu+ | Leu− | Leu+ | Leu− | Leu+ | Leu− | Leu+ | Leu− |
| | rHA+ | rHA+ | rHA− | rHA− | rHA+ | rHA+ | rHA− | rHA− |
| pDB2244 | 100 | 0 | 0 | 0 | | | | |
| pDB2976 | 7 | 0 | 47 | 46 | 97 | 0 | 3 | 0 |
| pDB2978 | 86 | 0 | 0 | 14 | 100 | 0 | 0 | 0 |
| pDB2980 | 98 | 0 | 0 | 2 | 100 | 0 | 0 | 0 |
| pDB2977 | 0 | 0 | 4 | 96 | 100 | 0 | 0 | 0 |
| pDB2979 | 69 | 0 | 6 | 25 | 100 | 0 | 0 | 0 |
| pDB2981 | 85 | 0 | 0 | 15 | 92 | 0 | 0 | 8 |

These data indicate plasmid retention is increased when the PDI1 gene is used as a selectable marker on a plasmid in a host strain having no chromosomally encoded PDI, even in a non-selective medium such as the exemplified rich medium.

1. A method for producing non-2 μm-family plasmid protein comprising:
(a) providing a host cell comprising a 2 μm-family plasmid, the plasmid comprising a gene encoding protein comprising the sequence of a chaperone protein and a gene encoding a non-2 μm-family plasmid protein;
(b) culturing the host cell in a culture medium under conditions that allow the co-expression of the gene encoding protein comprising the sequence of the chaperone protein and the gene encoding a non-2 μm-family plasmid protein; and
(c) purifying the thus expressed non-2 μm-family plasmid protein from the cultured host cell or the culture medium.

2. The method of claim 1 further comprising the step of formulating the purified non-2 μm-family plasmid protein with a carrier or diluent and optionally presenting the thus formulated protein in a unit dosage form.

3. A method of producing a non-2 μm-family plasmid protein comprising:
(a) providing a host cell comprising a 2 μm-family plasmid, the plasmid comprising a gene encoding protein comprising the sequence of a chaperone protein and a gene encoding a non-2 μm-family plasmid protein; and
(b) culturing the host cell in a culture medium under conditions that allow the co-expression of the gene encoding protein comprising the sequence of the chaperone protein and the gene encoding a non-2 μm-family plasmid protein,
wherein the non-2 μm-family is a fungal (preferably yeast) or vertebrate non-2 μm-family plasmid protein.

4. A 2 μm-family plasmid comprising a gene encoding a protein comprising the sequence of a chaperone protein and a gene encoding a non-2 μm-family plasmid protein, wherein if the plasmid is based on the 2 μm plasmid then it is a disintegration vector.

5. A method according to claim 1 wherein the chaperone has a sequence of a fungal chaperone (preferably a yeast chaperone) or a mammalian chaperone (preferably a human chaperone).

6. A method according to claim 1 wherein the chaperone comprises the sequence of a protein encoded by anyone of AHA1, CCT2, CCT3, CCT4, CCT5, CCT6, CCT7, CCT8, CNS1, CPR3, CPR6, EPS1, EPO1, EUG1, FMO1, HCH1, HSP10, HSP12, HSP104, HSP26, HSP30, HSP42, HSP60, HSP78, HSP82, JEM1, MDJ1, MDJ2, MPD1, MPD2, PDI1, PFD1, ABC1, APJ1, ATP1, ATP12, BTT1, CDC37, CPR7, HSC82, KAR2, LHS1, MGE1, MRS11, NOB1, ECM10, SSA1, SSA2, SSA3, SSA4, SSC1, SSE2, SIL1, SLS1, UBI4, ORM1, ORM2, PER1, PTC2, PSE1 and HAC1 or truncated intronless HAC1.

7. A method according to claim 1 wherein the chaperone is protein disulphide isomerase, or comprises the sequence of a protein encoded by PSE1, ORM2 or SSA1 or a variant or fragment thereof.

8. A method according to claim 1, wherein the host cell also expresses a second recombinant gene encoding a chaperone that is different to the first chaperone encoded by the plasmid.

9. A method according to claim 8 wherein the second recombinant gene encoding a chaperone is chromosomally integrated.

10. A method according to claim 1 wherein the plasmid comprises two different genes encoding different chaperones, one of which gene is the second recombinant gene encoding a chaperone as defined by claim 8.

11. A method according to claim 8 wherein one of the chaperones is protein disulphide isomerase.

12. A method according to claim 8 wherein one of the-chaperones is ORM2.

13. A method according to claim 8 wherein the two chaperones are protein disulphide isomerase and ORM2.

14. A method according to claim 1 wherein the non-2 μm-family plasmid protein comprises a leader sequence effective to cause secretion in yeast.

15. A method according to claim 1 wherein the non-2 μm-family plasmid protein is a eukaryotic protein, or a fragment or variant thereof, preferably a vertebrate or a fungal (such as a yeast) protein.

16. A method according to claim 1 wherein the non-2 μm-family plasmid protein is a commercially useful protein.

17. A method according to claim 1 wherein the non-2 μm-family plasmid protein comprises a sequence selected from albumin, a monoclonal antibody, an etoposide, a serum protein (such as a blood clotting factor), antistasin, a tick anticoagulant peptide, transferrin, lactoferrin, endostatin, angiostatin, collagens, immunoglobulins, or Immunoglobulin-based molecules or fragment of either (e.g. a dAb, Fab′ fragments, F (ab′)₂, scAb, scFv or scFv fragment), a Kunitz domain protein interferons, interleukins, IL10, IL11, IL2, interferon α species and sub-species, interferon β species and sub-species interferon γ species and sub-species, leptin, CNTF, CNTF_{Ax15} (Axokine™), IL1-receptor antagonist, erythropoietin (EPO) and EPO mimics, thrombopoietin (TPO) and TPO mimics, prosaptide, cyanovirin-N, 5-helix, T20 peptide, T1249 peptide, HIV gp41, HIV gp120, urokinase, prourokinase, tPA, hirudin, platelet derived growth factor, parathyroid hormone, proinsulin, insulin, glucagon, glucagon-like peptides, insulin-like growth factor, calcitonin, growth hormone, transforming growth factor β, tumour necrosis factor, G-CSF, GM-CSF, M-CSF, FGF, coagulation factors in both pre and active forms, including but not limited to plasminogen, fibrinogen, thrombin, pre-thrombin, pro-thrombin, von Willebrand's factor, α₁-antitrypsin, plasminogen activators, Factor VII, Factor VIII, Factor IX, Factor X and Factor XIII, nerve growth factor, LACI, platelet-derived endothelial cell growth factor (PD-ECGF), glucose oxidase, serum cholinesterase, aprotinin, amyloid precursor protein, inter-alpha trypsin inhibitor, antithrombin III, apo-lipoprotein species, Protein C, Protein S, or a variant or fragment of any of the above.

18. A method according to claim 1 wherein the non-2 μm-family plasmid protein comprises the sequence of albumin or a variant or fragment thereof.

19. A method according to claim 1 wherein the non-2 μm-family plasmid protein comprises the sequence of a transferrin family member, preferably transferrin or lactoferrin, or a variant or fragment thereof.

20. A method according to claim 1 wherein the non-2 μm-family plasmid protein comprises a fusion protein, such as a fusion protein of albumin or a transferrin family member or a variant or fragment of either, fused directly or indirectly to the sequence of another protein.

21. A host cell comprising a plasmid as defined by claim 4.

22. A host cell according to claim 21 wherein a chaperone encoded by the plasmid is an essential gene.

23. A host cell according to claim 22 wherein, in the absence of the plasmid, the host cell does not produce the chaperone.

24. A host cell according to claim 21 wherein the host cell is a yeast cell.

25. A host cell according to claim 24 in which the plasmid is based on pSR1, pSB3 or pSB4 and the yeast cell is Zygosaccharomyces rouxii, the plasmid is based on pSB1 or pSB2 and the yeast cell is Zygosaccharomyces bailli, the plasmid is based on pSM1 and the yeast cell is Zygosaccharomyces fermentati, the plasmid is based on pKD1 and the yeast cell is Kluyvejomyces drosophilarum, the plasmid is based on pPM1 and the yeast cell is Pichia menabranaefaciens, or the plasmid is based on the 2 μm plasmid and the yeast cell is Saccharomyces cerevisiae or Saccharomyces carlsbergensis.

26. A host cell according to claim 25 in which the plasmid is based on the 2 μm plasmid and the yeast cell is Saccharomyces cerevisiae or Saccharomyces carlsbergensis.

27. A method according to claim 1 wherein the host cell is a host cell as defined by claim 21.

28. A method according to claim 27 wherein the host cell is a host cell as defined by claim 23.

29. A method according to claim 27 wherein the step (b) involves culturing the host cell in non-selective media, such as a rich media.

30. A method for producing non-2 μm-family plasmid protein comprising:
(a) providing a host cell comprising a first recombinant gene encoding a protein comprising the sequence of a first chaperone protein, a second recombinant gene encoding a protein comprising the sequence of a second chaperone protein and a third recombinant gene encoding a non-2 μm-family plasmid protein, wherein the first and second chaperones are different;
(b) culturing the host cell in a culture medium under conditions that allow the expression of the first, second and third genes; and
(c) optionally purifying the thus expressed non-2 μm-family plasmid protein from the cultured host cell or the culture medium; and
(d) optionally, lyophilising the thus purified protein.

31. The method of claim 30 further comprising the step of formulating the purified non-2 μm-family plasmid protein with a carrier or diluent and optionally presenting the thus formulated protein in a unit dosage form.

32. A method according to claim 30 wherein the first and second chaperones comprise the sequence of a protein encoded by any one of AHA1, CCT2, CCT3, CCT4, CCT5, CCT6, CCT7, CCT8, CNS1, CPR3, CPR6, EPS1, ERO1, EUG1, FMO1, HCH1, HSP10, HSP12, HSP104, HSP26, HSP30, HSP42, HSP60, HSP78, HSP82, JEM1, MDJ1, MDJ2, MPD1, MPD2, PDI1, PFD1, ABC1, APJ1, ATP11, ATP12, BTT1, CDC37, CPR7, HSC82, KAR2, LHS1, MGE1, MRS11, NOB1, ECM10, SSA1, SSA2, SSA3, SSA4, SSC1, SSE2, SIL1, SLS1, UB14, ORM1, ORM2, PER1, PTC2, PSE1 and HAC1 or truncated intronless HAC1.

33. A method according to claim 30 wherein the first chaperone is protein disulphide isomerase.

34. A method according to claim 30 wherein the second chaperone is ORM2.

35. A method according to claim 30 wherein at least one of the first or second chaperones is encoded by a chromosomally integrated recombinant gene.

36. A method according to claim 30 wherein at least one of the first or second chaperones is encoded by a gene on a plasmid.

37. A method according to claim 36 wherein the plasmid is a plasmid as defined by claim 4.

38. A host cell comprising a first recombinant gene encoding a protein comprising the sequence of protein disulphide isomerase (PDI) and a second recombinant gene encoding a protein comprising the sequence of a transferrin-based protein.

39. (canceled)

40. A host cell according to claim 38 wherein the transferrin-based protein comprises the sequence of transferrin or any other member of the transferrin family (e.g. lactoferrin), a variant or fragment thereof or a fusion protein comprising transferrin, a variant or fragment thereof.

41. A host cell according to claim 38 wherein the first recombinant gene encoding a protein comprising the sequence of protein disulphide isomerase (PDI) is provided on a plasmid.

42. A host cell according to claim 41 wherein the plasmid is a 2 μm-family plasmid.

43. A host cell according to claim 38 wherein the first recombinant gene encoding a protein comprising the sequence of protein disulphide isomerase (PDI) is chromosomally integrated.

44. A host cell according to claim 43 wherein the first recombinant gene encoding a protein comprising the sequence of protein disulphide isomerase (PDI) is chromosomally integrated at the locus of an endogenously encoded PDI gene, preferably without disrupting the expression of the endogenous PDI gene.

45. A host cell according to claim 38 wherein the second recombinant gene encoding a protein comprising the sequence of a transferrin-based protein is provided on a plasmid.

46. A host cell according to claim 45 wherein the plasmid is a 2 μm-family plasmid.

47. A host cell according to claim 38 wherein the second recombinant gene encoding a protein comprising the sequence of a transferrin-based protein is chromosomally integrated.

48. A host cell according to claim 47 wherein the second recombinant gene encoding a protein comprising the sequence of a transferrin-based protein is chromosomally integrated at the locus of an endogenously encoded PDI gene, preferably without disrupting the expression of the endogenous PDI gene.

49. A method for producing non-2 μm-family plasmid protein comprising:
(a) providing a host cell comprising a first recombinant gene encoding a protein comprising the sequence of ORM2 or a variant or fragment thereof and a second recombinant gene encoding a non-2 μm-family plasmid protein; and
(b) culturing the host cell in a culture medium under conditions that allow the expression of the first and second genes.

50. The method of claim 49 further comprising the step of formulating the purified non-2 μm-family plasmid protein with a carrier or diluent and optionally presenting the thus formulated protein in a unit dosage form.

51. A method according to claim 49 wherein the first recombinant gene encoding a protein comprising the sequence of ORM2 or a variant or fragment thereof is integrated into the chromosome of the host cell.

52. A method according to claim 49 wherein the first recombinant gene encoding a protein comprising the sequence of ORM2 or a variant or fragment thereof is located on a plasmid.

53. A host cell comprising first recombinant gene encoding a protein comprising the sequence of ORM2 or a variant or fragment thereof and a second recombinant gene encoding a non-2 μm-family plasmid protein.

54. (canceled)

55. A plasmid comprising a first recombinant gene encoding a protein comprising the sequence of ORM2 or a variant or fragment thereof and a second recombinant gene encoding a non-2 μm-family plasmid protein.

56. A plasmid according to claim 55 which is a 2 μm-family plasmid.

57. A method according to claim 49 wherein the non-2 μm-family plasmid protein is as defined in claim 14.

58. A host cell comprising a plasmid, the plasmid comprising a gene that encodes an essential chaperone wherein, in the absence of the plasmid, the host cell is unable to produce the chaperone, the plasmid further comprising a recombinant gene encoding a non-2 μm-family plasmid protein, such as a non-2 μm-family plasmid protein as defined in claim 14.

59. A host cell according to claim 58 wherein, in the absence of the plasmid, the host cell is inviable.

60. The host cell of claim 58 wherein the chaperone is protein disulphide isomerase.

61. A plasmid comprising, as the sole selectable marker, a gene encoding an essential chaperone.

62. The plasmid of claim 61 further comprising a gene encoding a non-2 μm-family plasmid protein, such as a non-2 μm-family plasmid protein as defined in claim 14.

63. The plasmid of claim 61 which is a 2 m-family plasmid.

64. A method for producing a non-2 μm-family plasmid protein comprising the steps of:
(a) providing a host cell as defined by claim 58; and
(b) culturing the host cell in a culture medium under conditions that allow the expression of the essential chaperone and the non-2 μm-family plasmid protein.

65. The method of claim 64 wherein the host cell comprises a plasmid as defined by claim 61.

66. The method of claim 64 wherein step (b) is performed by culturing the host cell in a non-selective medium, such as a rich or complex medium.

67. (canceled)

68. (canceled)

69. A method for increasing the expression of a non-2 μm-family plasmid protein in a host cell comprising
providing a polynucleotide comprising a promoter operably connected to a coding sequence encoding a chaperone; and
expressing the polynucleotide sequence within the host cell,
wherein the promoter is characterised in that it achieves a lower level of expression of the chaperone than would be achieved if the coding sequence were to be operably connected to its naturally occurring promoter.

70. A method for producing a non-2 μm-family plasmid protein comprising the steps of:
(a) providing a host cell comprising a recombinant gene that comprises the sequence of a promoter operably connected to a coding sequence encoding a chaperone, the promoter being characterised in that it achieves a lower level of expression of the chaperone than would be achieved if the coding sequence were to be operably connected to its naturally occurring promoter, and the host cell further comprising a recombinant gene encoding a non-2 μm-family plasmid protein;
(b) culturing the host cell under conditions that allow the expression of the chaperone and the non-2 μm-family plasmid protein.

71. The method of claim 1 further comprising the step of lyophilising the thus purified protein.

72. The method of claim 49 further comprising the step of purifying the thus expressed non-2 μm-family plasmid protein from the cultured host cell or the culture medium.

73. The method of claim 72 further comprising the step of lyophilising the thus purified protein.

74. The method of claim 72 further comprising the step of formulating the purified non-2 μm-family plasmid protein with a carrier or diluent.

75. The method of claim 74 further comprising the step of presenting the thus formulated protein in a unit dosage form.
